(19) United States
(12) Patent Application Publication
(10) Pub. No.: US 2023/0348579 A1
(43) Pub. Date: Nov. 2, 2023

(54) DOSAGE AND ADMINISTRATION OF ANTI-C5 ANTIBODIES FOR TREATING C5-MEDIATED GLOMERULAR NEPHRITIS (GN), INCLUDING LUPUS NEPHRITIS (LN) AND/OR IGA NEPHROPATHY (IGAN)

(71) Applicant: Alexion Pharmaceuticals, Inc., Boston, MA (US)

(72) Inventor: Nader Najafian, Chestnut Hill, MA (US)

(73) Assignee: Alexion Pharmaceuticals, Inc., Boston, MA (US)

(21) Appl. No.: 18/026,698

(22) PCT Filed: Sep. 16, 2021
(86) PCT No.: PCT/US2021/050590

§ 371 Date: Mar. 16, 2023

Related U.S. Application Data
(60) Provisional application No. 63/081,182, filed on Sep. 21, 2020.

(51) Int. Cl. C07K 16/18; A61K 45/06; A61P 13/12

(52) U.S. Cl. CPC C07K 16/18; A61K 45/06; A61P 13/12; A61K 2039/545

(57) ABSTRACT

Provided are dosages and methods for clinical treatment of C5-mediated glomerular nephritis (GN), including lupus nephritis (LN) and immunoglobulin A nephropathy (IgAN), in human patients using an anti C5 antibody, or antigen binding fragment thereof (e.g., such as ravulizumab (ULTOMIRIS®)), optionally together with background therapy for treating LN (e.g., an immuno-suppressant) or background therapy for treating IgAN (e.g., renin-angiotensin system (RAS) inhibiting medication).

### CROSS REFERENCE TO RELATED APPLICATIONS

This application claims the benefit of U.S. Provisional Application No. 63/081,182, filed Sep. 21, 2020, the contents of which is incorporated by reference herein in its entirety.

### SEQUENCE LISTING

The instant application contains a Sequence Listing which has been submitted electronically in ASCII format and is hereby incorporated by reference in its entirety. Said ASCII copy, created on Sep. 14, 2021, is named 0640WO_SL.txt and is 58,872 bytes in size.

### BACKGROUND

Chronic kidney disease (CKD) has become a worldwide public health issue due to its high incidence, poor prognosis, and substantial economic burden. When not properly diagnosed and managed, CKD can lead to many adverse outcomes, such as end-stage renal disease (ESRD). Despite advances in immunosuppressive treatments, certain types of C5-mediated glomerular nephritis (GN), such as lupus nephritis (LN) and immunoglobulin A nephropathy (IgAN) continue to respond poorly to treatment, resulting over time in CKD. No disease-specific therapies are currently available, therefore effective treatments for managing GN, including LN and IgAN, represent a high unmet medical need. Accordingly, it is an object of the present disclosure to provide improved methods for treating patients with GN, such as LN and/or IgAN.

### SUMMARY

Provided herein are compositions and methods for treating C5-mediated glomerular nephritis (GN), including lupus nephritis (LN) and immunoglobulin A nephropathy (IgAN), in a human patient (e.g., an adult patient), comprising administering to the patient an anti-C5 antibody, or antigen binding fragment thereof, wherein the anti-C5 antibody, or antigen binding fragment thereof, is administered (or is for administration) according to a particular clinical dosage regimen (e.g., at a particular dose amount and according to a specific dosing schedule).

An exemplary anti-C5 antibody is ravulizumab (ULTOMIRIS®) comprising the heavy and light chains having the sequences shown in SEQ ID NOs:14 and 11, respectively, or antigen binding fragments and variants thereof. In other embodiments, the antibody comprises the heavy and light chain complementarity determining regions (CDRs) or variable regions (VRs) of ravulizumab. Accordingly, in one embodiment, the antibody comprises the CDR1, CDR2 and CDR3 domains of the heavy chain variable (VH) region of ravulizumab having the sequence shown in SEQ ID NO:12, and the CDR1, CDR2 and CDR3 domains of the light chain variable (VL) region of ravulizumab having the sequence shown in SEQ ID NO:8. In another embodiment, the antibody comprises CDR1, CDR2 and CDR3 heavy chain sequences as set forth in SEQ ID NOs:19, 18 and 3, respectively, and CDR1, CDR2 and CDR3 light chain sequences as set forth in SEQ ID NOs:4, 5 and 6, respectively. In another embodiment, the antibody comprises VH and VL regions having the amino acid sequences set forth in SEQ ID NO:12 and SEQ ID NO:8, respectively. In another embodiment, the antibody comprises a heavy chain constant region as set forth in SEQ ID NO:13.

In another embodiment, the antibody comprises a variant human Fc constant region that binds to human neonatal Fc receptor (FcRn), wherein the variant human Fc CH3 constant region comprises Met429Leu and Asn435Ser substitutions at residues corresponding to methionine 428 and asparagine 434 of a native human IgG Fc constant region, each according to the EU numbering convention.

In another embodiment, the antibody comprises CDR1, CDR2 and CDR3 heavy chain sequences as set forth in SEQ ID NOs:19, 18 and 3, respectively, and CDR1, CDR2 and CDR3 light chain sequences as set forth in SEQ ID NOs:4, 5 and 6, respectively and a variant human Fc constant region that binds to human neonatal Fc receptor (FcRn), wherein the variant human Fc CH3 constant region comprises Met429Leu and Asn435Ser substitutions at residues corresponding to methionine 428 and asparagine 434 of a native human IgG Fc constant region, each according to the EU numbering convention.

In another embodiment, the anti-C5 antibody comprises the heavy and light chain CDRs or variable regions of the BNJ421 antibody (described in WO2015134894 and U.S. Pat. No. 9,079,949). In another embodiment, the anti-C5 antibody comprises the heavy and light chain CDRs or variable regions of the 7086 antibody (see U.S. Pat. Nos. 8,241,628 and 8,883,158). In another embodiment, the anti-C5 antibody comprises the heavy and light chain CDRs or variable regions of the 8110 antibody (see U.S. Pat. Nos. 8,241,628 and 8,883,158). In another embodiment, the anti-C5 antibody comprises the heavy and light chain CDRs or variable regions of the 305LO5 antibody (see U.S. Pat. No. 9,765,135). In another embodiment, the anti-C5 antibody comprises the heavy and light chain CDRs or variable regions of the SKY59 antibody. In another embodiment, the anti-C5 antibody comprises the heavy and light chain CDRs or variable regions of the REGN3918 antibody.

In another embodiment, the antibody competes for binding with, and/or binds to the same epitope on C5 as any of the above-mentioned antibodies. In another embodiment, the antibody has at least about 90% variable region amino acid sequence identity to any of the above-mentioned antibodies (e.g., at least about 90%, 95% or 99% variable region identity with SEQ ID NO:12 or SEQ ID NO:8).

In another embodiment, the antibody binds to human C5 at pH 7.4 and 25° C. with an affinity dissociation constant (K_{D}) that is in the range 0.1 nM≤K_{D}≤1 nM. In another embodiment, the antibody binds to human C5 at pH 7.4 and 25° C. with an affinity dissociation constant (K_{D}) of about 0.5 nM. In another embodiment, the antibody binds to human C5 at pH 6.0 and 25° C. with a K_{D}≥10 nM. In another embodiment, the antibody binds to human C5 at pH 6.0 and 25° C. with a K_{D} of about 22 nM. In yet another embodiment, the [(K_{D} of the antibody or antigen-binding fragment thereof for human C5 at pH 6.0 and at 25° C.)/(K_{D} of the antibody or antigen-binding fragment thereof for human C5 at pH 7.4 and at 25° C.)] of the antibody is greater than 25.

In one embodiment, the dose of the anti-C5 antibody, or antigen binding fragment thereof, is based on the weight of the patient. In one embodiment, for example, 900 mg, 2400 mg, or 3000 mg of the anti-C5 antibody, or antigen binding fragment thereof, is administered to a patient weighing ≥40 to <60 kg. In another embodiment, 900 mg, 2700 mg, or 3900 mg of the anti-C5 antibody, or antigen binding fragment thereof, is administered to a patient weighing ≥60 to <100 kg. In another embodiment, 900 mg, 3000 mg, or 5400 mg of the anti-C5 antibody, or antigen binding fragment thereof, is administered to a patient weighing ≥100 kg. In certain embodiments, dosage regimens are adjusted to provide the optimum desired response (e.g., an effective response).

In another embodiment, the anti-C5 antibody, or antigen binding fragment thereof, is administered for one or more administration cycles. In one embodiment, the treatment (e.g., administration cycle) is 26 weeks. In one embodiment, the anti-C5 antibody, or antigen binding fragment thereof, is administered once on Days 1, 15, 71, 127, and 183 (e.g., of the administration cycle). In another embodiment, the anti-C5 antibody, or antigen binding fragment thereof, is administered for up to two years (e.g., at a dose of 900 mg, 2400 mg, 2700 mg, or 3000 mg).

In another embodiment, a method of treating a human patient with C5-mediated GN is provided, the method comprising administering to the patient (e.g., during an administration cycle) an effective amount of an anti-C5 antibody or antigen binding fragment thereof, comprising CDR1, CDR2 and CDR3 heavy chain sequences as set forth in SEQ ID NOs:19, 18 and 3, respectively, and CDR1, CDR2 and CDR3 light chain sequences as set forth in SEQ ID NOs:4, 5 and 6, respectively, wherein the anti-C5 antibody or antigen binding fragment thereof is administered:
(a) once on Day 1 at a dose of 2400 mg, followed by a dose of 3000 mg at Week 2 and once every eight weeks thereafter to a patient weighing ≥40 to <60 kg;
(b) once on Day 1 at a dose of 2700 mg, followed by a dose of 3900 mg at Week 2 and once every eight weeks thereafter to a patient weighing ≥60 to <100 kg; or
(c) once on Day 1 at a dose of 3000 mg, followed by a dose of 5400 mg at Week 2 and once every eight weeks thereafter to a patient weighing ≥100 kg.

In another embodiment, a method of treating a human patient with C5-mediated GN is provided, the method comprising further administering to the patient the anti-C5 antibody, or antigen binding fragment thereof:
(a) once on Day 183 at a dose of 900 mg, followed by a dose of 3000 mg at Day 197 and once every eight weeks thereafter to a patient weighing ≥40 to <60 kg;
(b) once on Day 183 at a dose of 900 mg, followed by a dose of 3900 mg at Day 197 and once every eight weeks thereafter to a patient weighing ≥60 to <100 kg; or
(c) once on Day 183 at a dose of 900 mg, followed by a dose of 5400 mg at Day 197 and once every eight weeks thereafter to a patient weighing ≥100 kg.
   In one embodiment the C5-mediated GN is LN. In another embodiment, the C5-mediated GN is IgAN.

In another embodiment, a method of treating a human patient with C5-mediated GN is provided, the method comprising administering to the patient (e.g., during an administration cycle) an effective amount of an anti-C5 antibody or antigen binding fragment thereof, comprising CDR1, CDR2 and CDR3 heavy chain sequences as set forth in SEQ ID NOs:19, 18 and 3, respectively, and CDR1, CDR2 and CDR3 light chain sequences as set forth in SEQ ID NOs:4, 5 and 6, respectively, and a variant human Fc constant region that binds to human neonatal Fc receptor (FcRn), wherein the variant human Fc constant region comprises Met429Leu and Asn435Ser substitutions at residues corresponding to methionine 428 and asparagine 434 of a native human IgG Fc constant region, each in EU numbering, wherein the anti-C5 antibody or antigen binding fragment thereof is administered:
(a) once on Day 1 at a dose of 2400 mg, followed by a dose of 3000 mg at Week 2 and once every eight weeks thereafter to a patient weighing ≥40 to <60 kg;
(b) once on Day 1 at a dose of 2700 mg, followed by a dose of 3900 mg at Week 2 and once every eight weeks thereafter to a patient weighing ≥60 to <100 kg; or
(c) once on Day 1 at a dose of 3000 mg, followed by a dose of 5400 mg at Week 2 and once every eight weeks thereafter to a patient weighing ≥100 kg.

In another embodiment, a method of treating a human patient with C5-mediated GN is provided, the method comprising further administering to the patient the anti-C5 antibody, or antigen binding fragment thereof:
(a) once on Day 183 at a dose of 900 mg, followed by a dose of 3000 mg at Day 197 and once every eight weeks thereafter to a patient weighing ≥40 to <60 kg;
(b) once on Day 183 at a dose of 900 mg, followed by a dose of 3900 mg at Day 197 and once every eight weeks thereafter to a patient weighing ≥60 to <100 kg; or
(c) once on Day 183 at a dose of 900 mg, followed by a dose of 5400 mg at Day 197 and once every eight weeks thereafter to a patient weighing ≥100 kg.
   In one embodiment the C5-mediated GN is LN. In another embodiment, the C5-mediated GN is IgAN.

In another embodiment, a method of treating a human patient with C5-mediated GN is provided, the method comprising administering to the patient (e.g., during an administration cycle) an effective amount of an anti-C5 antibody or antigen binding fragment thereof, comprising CDR1, CDR2 and CDR3 heavy chain sequences as set forth in SEQ ID NOs:19, 18 and 3, respectively, and CDR1, CDR2 and CDR3 light chain sequences as set forth in SEQ ID NOs:4, 5 and 6, respectively, wherein the anti-C5 antibody is administered to a patient weighing ≥40 to <60 kg once on Day 1 at a dose of 2400 mg, followed by a dose of 3000 mg at Week 2 and once every eight weeks thereafter.

In another embodiment, a method of treating a human patient with C5-mediated GN is provided, the method comprising administering to the patient (e.g., during an administration cycle) an effective amount of an anti-C5 antibody or antigen binding fragment thereof, comprising CDR1, CDR2 and CDR3 heavy chain sequences as set forth in SEQ ID NOs:19, 18 and 3, respectively, and CDR1, CDR2 and CDR3 light chain sequences as set forth in SEQ ID NOs:4, 5 and 6, respectively, wherein the anti-C5 antibody is administered to a patient weighing ≥60<100 kg once on Day 1 at a dose of 2700 mg, followed by a dose of 3900 mg at Week 2 and once every eight weeks thereafter.

In another embodiment, a method of treating a human patient with C5-mediated GN is provided, the method comprising administering to the patient (e.g., during an administration cycle) an effective amount of an anti-C5 antibody or antigen binding fragment thereof, comprising CDR1, CDR2 and CDR3 heavy chain sequences as set forth in SEQ ID NOs:19, 18 and 3, respectively, and CDR1, CDR2 and CDR3 light chain sequences as set forth in SEQ ID NOs:4, 5 and 6, respectively, wherein the anti-C5 antibody is administered to a patient weighing ≥100 kg once on Day 1 at a dose of 3000 mg, followed by a dose of 5400 mg at Week 2 and once every eight weeks thereafter to a patient weighing ≥100 kg.

In another embodiment, a method of treating a human patient with C5-mediated GN is provided, the method comprising administering an anti-C5 antibody, or antigen binding fragment thereof, to a patient weighing ≥40 to <60 kg:
(a) once on Day 1 at a dose of 2400 mg, followed by a dose of 3000 mg at Week 2 and once every eight weeks thereafter; and
(b) once on Day 183 at a dose of 900 mg, followed by a dose of 3000 mg on Day 197 and once every eight weeks thereafter.

In another embodiment, a method of treating a human patient with C5-mediated GN is provided, the method comprising administering an anti-C5 antibody, or antigen binding fragment thereof, to a patient weighing ≥60<100 kg:
(a) once on Day 1 at a dose of 2700 mg, followed by a dose of 3900 mg at Week 2 and once every eight weeks thereafter; and
(b) once on Day 183 at a dose of 900 mg, followed by a dose of 3900 mg on Day 197 and once every eight weeks thereafter.

In another embodiment, a method of treating a human patient with C5-mediated GN is provided, the method comprising administering an anti-C5 antibody, or antigen binding fragment thereof, to a patient weighing ≥100 kg:
(a) once on Day 1 at a dose of 3000 mg, followed by a dose of 5400 mg at Week 2 and once every eight weeks thereafter; and
(b) once on Day 183 at a dose of 900 mg, followed by a dose of 5400 mg on Day 197 and once every eight weeks thereafter.

In another embodiment, a method of treating a human patient with LN is provided, the method comprising administering to the patient (e.g., during an administration cycle) an effective amount of an anti-C5 antibody or antigen binding fragment thereof, comprising CDR1, CDR2 and CDR3 heavy chain sequences as set forth in SEQ ID NOs:19, 18 and 3, respectively, and CDR1, CDR2 and CDR3 light chain sequences as set forth in SEQ ID NOs:4, 5 and 6, respectively, wherein the anti-C5 antibody or antigen binding fragment thereof is administered:
(a) once on Day 1 at a dose of 2400 mg, followed by a dose of 3000 mg at Week 2 and once every eight weeks thereafter to a patient weighing ≥40 to <60 kg;
(b) once on Day 1 at a dose of 2700 mg, followed by a dose of 3900 mg at Week 2 and once every eight weeks thereafter to a patient weighing ≥60 to <100 kg; or
(c) once on Day 1 at a dose of 3000 mg, followed by a dose of 5400 mg at Week 2 and once every eight weeks thereafter to a patient weighing ≥100 kg.

In another embodiment, a method of treating a human patient with LN is provided, the method comprising administering to the patient (e.g., during an administration cycle) an effective amount of an anti-C5 antibody or antigen binding fragment thereof, comprising CDR1, CDR2 and CDR3 heavy chain sequences as set forth in SEQ ID NOs:19, 18 and 3, respectively, and CDR1, CDR2 and CDR3 light chain sequences as set forth in SEQ ID NOs:4, 5 and 6, respectively, and a variant human Fc constant region that binds to human neonatal Fc receptor (FcRn), wherein the variant human Fc constant region comprises Met429Leu and Asn435Ser substitutions at residues corresponding to methionine 428 and asparagine 434 of a native human IgG Fc constant region, each in EU numbering, wherein the anti-C5 antibody or antigen binding fragment thereof is administered:
(a) once on Day 1 at a dose of 2400 mg, followed by a dose of 3000 mg at Week 2 and once every eight weeks thereafter to a patient weighing ≥40 to <60 kg;
(b) once on Day 1 at a dose of 2700 mg, followed by a dose of 3900 mg at Week 2 and once every eight weeks thereafter to a patient weighing ≥60 to <100 kg; or
(c) once on Day 1 at a dose of 3000 mg, followed by a dose of 5400 mg at Week 2 and once every eight weeks thereafter to a patient weighing ≥100 kg.

In another embodiment, a method of treating a human patient with LN is provided, the method comprising administering to the patient (e.g., during an administration cycle) an effective amount of an anti-C5 antibody or antigen binding fragment thereof, comprising CDR1, CDR2 and CDR3 heavy chain sequences as set forth in SEQ ID NOs:19, 18 and 3, respectively, and CDR1, CDR2 and CDR3 light chain sequences as set forth in SEQ ID NOs:4, 5 and 6, respectively, wherein the anti-C5 antibody is administered to a patient weighing ≥40 to <60 kg once on Day 1 at a dose of 2400 mg, followed by a dose of 3000 mg at Week 2 and once every eight weeks thereafter.

In another embodiment, a method of treating a human patient with LN is provided, the method comprising administering to the patient (e.g., during an administration cycle) an effective amount of an anti-C5 antibody or antigen binding fragment thereof, comprising CDR1, CDR2 and CDR3 heavy chain sequences as set forth in SEQ ID NOs:19, 18 and 3, respectively, and CDR1, CDR2 and CDR3 light chain sequences as set forth in SEQ ID NOs:4, 5 and 6, respectively, wherein the anti-C5 antibody is administered to a patient weighing ≥60<100 kg once on Day 1 at a dose of 2700 mg, followed by a dose of 3900 mg at Week 2 and once every eight weeks thereafter.

In another embodiment, a method of treating a human patient with LN is provided, the method comprising administering to the patient (e.g., during an administration cycle) an effective amount of an anti-C5 antibody or antigen binding fragment thereof, comprising CDR1, CDR2 and CDR3 heavy chain sequences as set forth in SEQ ID NOs:19, 18 and 3, respectively, and CDR1, CDR2 and CDR3 light chain sequences as set forth in SEQ ID NOs:4, 5 and 6, respectively, wherein the anti-C5 antibody is administered to a patient weighing ≥100 kg once on Day 1 at a dose of 3000 mg, followed by a dose of 5400 mg at Week 2 and once every eight weeks thereafter to a patient weighing ≥100 kg.

In another embodiment, a method of treating a human patient with immunoglobulin A nephropathy (IgAN), is provided, the method comprising administering to the patient (e.g., during an administration cycle) an effective amount of an anti-C5 antibody or antigen binding fragment thereof, comprising CDR1, CDR2 and CDR3 heavy chain sequences as set forth in SEQ ID NOs:19, 18 and 3, respectively, and CDR1, CDR2 and CDR3 light chain sequences as set forth in SEQ ID NOs:4, 5 and 6, respectively, wherein the anti-C5 antibody or antigen binding fragment thereof is administered:
(a) once on Day 1 at a dose of 2400 mg, followed by a dose of 3000 mg at Week 2 and once every eight weeks thereafter to a patient weighing ≥40 to <60 kg;
(b) once on Day 1 at a dose of 2700 mg, followed by a dose of 3900 mg at Week 2 and once every eight weeks thereafter to a patient weighing ≥60 to <100 kg; or
(c) once on Day 1 at a dose of 3000 mg, followed by a dose of 5400 mg at Week 2 and once every eight weeks thereafter to a patient weighing ≥100 kg.

In another embodiment, a method of treating a human patient with IgAN, is provided, the method comprising administering to the patient (e.g., during an administration cycle) an effective amount of an anti-C5 antibody or antigen binding fragment thereof, comprising CDR1, CDR2 and CDR3 heavy chain sequences as set forth in SEQ ID NOs:19, 18 and 3, respectively, and CDR1, CDR2 and CDR3 light chain sequences as set forth in SEQ ID NOs:4, 5 and 6, respectively, and a variant human Fc constant region that binds to human neonatal Fc receptor (FcRn), wherein the variant human Fc constant region comprises Met429Leu and Asn435Ser substitutions at residues corresponding to methionine 428 and asparagine 434 of a native human IgG Fc constant region, each in EU numbering, wherein the anti-C5 antibody or antigen binding fragment thereof is administered:
(a) once on Day 1 at a dose of 2400 mg, followed by a dose of 3000 mg at Week 2 and once every eight weeks thereafter to a patient weighing ≥40 to <60 kg;
(b) once on Day 1 at a dose of 2700 mg, followed by a dose of 3900 mg at Week 2 and once every eight weeks thereafter to a patient weighing ≥60 to <100 kg; or
(c) once on Day 1 at a dose of 3000 mg, followed by a dose of 5400 mg at Week 2 and once every eight weeks thereafter to a patient weighing ≥100 kg.

In another embodiment, a method of treating a human patient with IgAN, is provided, the method comprising further administering to the patient the anti-C5 antibody, or antigen binding fragment thereof:
(a) once on Day 183 at a dose of 900 mg, followed by a dose of 3000 mg at Day 197 and once every eight weeks thereafter to a patient weighing ≥40 to <60 kg;
(b) once on Day 183 at a dose of 900 mg, followed by a dose of 3900 mg at Day 197 and once every eight weeks thereafter to a patient weighing ≥60 to <100 kg; or
(c) once on Day 183 at a dose of 900 mg, followed by a dose of 5400 mg at Day 197 and once every eight weeks thereafter to a patient weighing ≥100 kg.

In another embodiment, a method of treating a human patient with IgAN is provided, the method comprising administering an anti-C5 antibody, or antigen binding fragment thereof, to a patient weighing ≥40 to <60 kg:
(a) once on Day 1 at a dose of 2400 mg, followed by a dose of 3000 mg at Week 2 and once every eight weeks thereafter; and
(b) once on Day 183 at a dose of 900 mg, followed by a dose of 3000 mg on Day 197 and once every eight weeks thereafter.

In another embodiment, a method of treating a human patient with IgAN is provided, the method comprising administering an anti-C5 antibody, or antigen binding fragment thereof, to a patient weighing ≥60<100 kg:
(a) once on Day 1 at a dose of 2700 mg, followed by a dose of 3900 mg at Week 2 and once every eight weeks thereafter; and
(b) once on Day 183 at a dose of 900 mg, followed by a dose of 3900 mg on Day 197 and once every eight weeks thereafter.

In another embodiment, a method of treating a human patient with IgAN is provided, the method comprising administering an anti-C5 antibody, or antigen binding fragment thereof, to a patient weighing ≥100 kg:
(a) once on Day 1 at a dose of 3000 mg, followed by a dose of 5400 mg at Week 2 and once every eight weeks thereafter; and
(b) once on Day 183 at a dose of 900 mg, followed by a dose of 5400 mg on Day 197 and once every eight weeks thereafter.

In one embodiment, the patient has not previously been treated with eculizumab. In another embodiment, the patient has previously been treated with eculizumab. In another embodiment, the patient has previously been treated with eculizumab and Day 1 (e.g., of the administration cycle) is two weeks or more from the patient's last dose of eculizumab.

In another embodiment, the patient is an LN patient who has been previously treated with a background therapy comprising an immunosuppressant, e.g., corticosteroids and mycophenolate mofetil.

In another embodiment, the patient is an IgAN patient who has previously been treated with a background therapy comprising a renin-angiotensin system (RAS) inhibiting medication, such as an angiotensin-converting enzyme (ACE) inhibitor or angiotensin II receptor blocker (ARB).

In another embodiment, the treatment further comprises administering one or more of the following to, e.g., an LN patient: pneumocystis pneumonia prophylaxis, an antimalarial agent (e.g., hydroxychloroquine), and/or an agent to treat osteoporosis (e.g., calcium carbonate or citrate, Vitamin D, and/or bisphosphonates).

In another aspect, the treatment regimens described are sufficient to maintain particular serum trough concentrations of the anti-C5 antibody or antigen binding fragment thereof. In one embodiment, for example, the treatment regimen maintains a serum trough concentration of the anti-C5 antibody or antigen binding fragment thereof of 50, 55, 60, 65, 70, 75, 80, 85, 90, 95, 100, 105, 110, 115, 120, 125, 130, 135, 140, 145, 150, 155, 160, 165, 170, 175, 180, 185, 190, 200, 205, 210, 215, 220, 225, 230, 240, 245, 250, 255, 260, 265, 270, 280, 290, 300, 305, 310, 315, 320, 325, 330, 335, 340, 345, 350, 355, 360, 365, 370, 375, 380, 385, 390, 395 or 400 μg/mL or greater. In one embodiment, the treatment regimen maintains a serum trough concentration of the anti-C5 antibody or antigen binding fragment thereof of 100 μg/mL or greater, 150 μg/mL or greater, 200 μg/mL or greater, 250 μg/mL or greater, or 300 μg/mL or greater. In another embodiment, the treatment maintains a serum trough concentration of the anti-C5 antibody or antigen binding fragment thereof of between 100 μg/mL and 200 μg/mL. In another embodiment, the treatment maintains a serum trough concentration of the anti-C5 antibody or antigen binding fragment thereof of about 175 μg/mL.

In another embodiment, to obtain an effective response, the anti-C5 antibody is administered to the patient in an amount and with a frequency to maintain at least 50 μg, 55 μg, 60 μg, 65 μg, 70 μg, 75 μg, 80 μg, 85 μg, 90 μg, 95 μg, 100 μg, 105 μg, 110 μg, 115 μg, 120 μg, 125 μg, 130 μg, 135 μg, 140 μg, 145 μg, 150 μg, 155 μg, 160 μg, 165 μg, 170 μg, 175 μg, 180 μg, 185 μg, 190 μg, 195 μg, 200 μg, 205 μg, 210 μg, 215 μg, 220 μg, 225 μg, 230 μg, 235 μg, 240 μg, 245 μg, 250 μg, 255 μg or 260 μg of antibody per milliliter of the patient's blood. In another embodiment, the anti-C5 antibody is administered to the patient in an amount and with a frequency to maintain between 50 μg and 250 μg of antibody per milliliter of the patient's blood. In another embodiment, the anti-C5 antibody is administered to the patient in an amount and with a frequency to maintain between 100 μg and 200 μg of antibody per milliliter of the patient's blood. In another embodiment, the anti-C5 antibody is administered to the patient in an amount and with a frequency to maintain about 175 μg of antibody per milliliter of the patient's blood.

In another embodiment, to obtain an effective response, the anti-C5 antibody is administered to the patient in an amount and with a frequency to maintain a minimum free C5 concentration. In one embodiment, for example, the anti-C5 antibody is administered to the patient in an amount and with a frequency to maintain a free C5 concentration of 0.5 μg/mL or less (e.g., 0.4 μg/mL, 0.3 μg/mL, 0.2 μg/mL, or 0.1 μg/mL or less).

The anti-C5 antibodies, or antigen binding fragments thereof, can be administered to a patient by any suitable means. In one embodiment, the antibodies are formulated for intravenous administration.

The efficacy of the treatment methods provided herein can be assessed using any suitable means. In one embodiment, the treatment results in a shift towards normal levels of one or more renal injury biomarkers selected from the group consisting of CD163, MCP-1, and EGF.

In another embodiment, the treatment results in a shift towards normal levels of one or more biomarkers selected from the group consisting of sC5b-9, Factor Ba, Factor Bb, C5a, C3c, C3, C4d, CD68, properdin, complement component 9 [C9], C1q, C5aR, and creatinine.

In another embodiment, the treatment results in a change in Estimated glomerular filtration rate (eGFR) compared to baseline.

In another embodiment, the treatment results in a change in serum albumin compared to baseline.

In another embodiment, the treatment results in a reduction in proteinuria compared to baseline. In another embodiment, the patient has an estimated glomerular filtration rate (eGFR)≥30 mL/min/1.73 m2 and proteinuria prior to treatment. In another embodiment, the proteinuria for an LN patient is a urine protein to creatinine ratio (UPCR)≥1 g/g from one 24-hr urine collection. In another embodiment, proteinuria for an IgAN patient is a mean protein≥1 g/24-hr from 2 valid 24-hr collections. In another embodiment, the treatment results in a 25%, 30%, 35%, 40%, 45%, 50%, 55%, 60%, 65%, 70%, 75%, 80%, 85%, 90%, or 95% reduction in proteinuria compared to baseline. In another embodiment, the reduction in proteinuria occurs at 6 weeks, 8 weeks, 10 weeks, 12 weeks, 14 weeks, 16 weeks, 18 weeks, 20 weeks, 22 weeks, 24 weeks, 26 weeks, 28 weeks, or 30 weeks after treatment compared to baseline. In another embodiment, proteinuria is measured by a complete 24-hour urine collection.

In another embodiment, the treatment results in a reduction or cessation in one or more of the following symptoms compared to baseline in an LN patient: foamy urine, proteinuria, edema, high blood pressure, kidney inflammation, kidney impairment, joint pain, joint swelling, muscle pain, fever with no known cause, high levels of creatinine in the blood, and/or a red rash.

In another embodiment, the treatment results the LN patient has an active flare prior to treatment.

In another embodiment, the treatment results in a Complete Renal Response (CRR) in an LN patient. In another embodiment, the CRR comprises:
(a) a decrease in mean urine protein-to-creatinine ratio (UPCR) to ≤0.5 g/g based on two 24-hour urine collections;
(b) an Estimated glomerular filtration rate (eGFR)>60 mL/min/1.73 m2 or no eGFR reduction>20% from the baseline value based on mean of 2 values; and
(c) no treatment failure.

In another embodiment, the treatment results in a Partial Renal Response (PRR) in an IgAN patient. In another embodiment, the PRR comprises:
(a) a decrease in UPCR>50% compared to the baseline value based on mean of two 24 hour urine collections;
(b) an Estimated glomerular filtration rate (eGFR)>60 mL/min/1.73 m2 or no eGFR reduction≥20% from the baseline value based on mean of 2 values; and
(c) no treatment failure.

In another embodiment, the treatment prevents a renal flare in an LN patient, wherein:
(a) renal flare for a patient who has achieved CRR is reproducible recurrence of proteinuria≥1 g/g; and
(b) renal flare for a patient who has not achieved CRR is:
   (i) a reproducible increase of serum creatinine>25% higher than baseline or above the upper limit of normal, including any one of the following:
      a. reproducible proteinuria≥75% higher than baseline;
      b. worsening active urinary sediment compared to baseline as defined by an increase of ≥5 RBCs/high power field (hpf) or new RBC casts (based on local laboratory results from at least 2 samples); and/or
      c. kidney biopsy newly conducted since the biopsy used for eligibility demonstrating LN Class III or IV activity;
   (ii) a reproducible doubling of the UPCR from a 24 hour urine collection compared with the lowest previous value obtained after the first dose of the anti-C5 antibody, or antigen binding fragment thereof.

In another embodiment, the treatment prevents an Extrarenal SLE Flare in a LN patient, wherein the Extrarenal SLE Flare comprises an increase in Systemic Lupus Erythematosus Disease Activity Index Safety of Estrogen in Lupus Erythematosus National Assessment (SELENA) Modification (SLEDAI-2K)>4 points that is not accounted for by proteinuria, hematuria, urinary cellular casts, hypocomplementemia, or an increase in anti-double-stranded DNA (anti-dsDNA) antibody level.

In another embodiment, the treatment results in Partial Remission (PR) in an IgAN patient. In another embodiment, the PR comprises mean proteinuria<1 g/24-hours based on 2 valid 24-hour urine collections.

In another embodiment, the treatment results in a reduction or cessation in one or more of the following symptoms in an IgAN patient compared to baseline: hematuria, dark brown or cola colored urine, edema, flank pain, hypertension, foamy urine, and/or proteinuria.

In another embodiment, the treatment results in an improvement in the patient's quality of life, as assessed by European Quality of Life Health 5-item questionnaire dimensions 5 level (EQ-5D-5L) and/or Short Form (36) Health Survey (SF-36) total score (e.g., for an LN and/or IgAN patient).

In another embodiment, the treatment results in an improvement in the patient's quality of life, as assessed by Functional Assessment of Chronic Therapy (FACIT)-Fatigue score (e.g., for an LN patient).

In another embodiment, the treatment results in terminal complement inhibition.

In another embodiment, the treatment results in a reduction in adverse events.

In another aspect, an anti-C5 antibody, or antigen binding fragment thereof, is provided, comprising CDR1, CDR2 and CDR3 domains of the heavy chain variable region having the sequence set forth in SEQ ID NO:12, and CDR1, CDR2 and CDR3 domains of the light chain variable region having the sequence set forth in SEQ ID NO:8, for administration:
(a) once on Day 1 at a dose of 2400 mg, followed by a dose of 3000 mg at Week 2 and once every eight weeks thereafter to a patient weighing ≥40 to <60 kg;
(b) once on Day 1 at a dose of 2700 mg, followed by a dose of 3900 mg at Week 2 and once every eight weeks thereafter to a patient weighing ≥60 to <100 kg; or
(c) once on Day 1 at a dose of 3000 mg, followed by a dose of 5400 mg at Week 2 and once every eight weeks thereafter to a patient weighing ≥100 kg.

In another aspect, an anti-C5 antibody, or antigen binding fragment thereof, is provided, comprising CDR1, CDR2 and CDR3 domains of the heavy chain variable region having the sequence set forth in SEQ ID NO:12, and CDR1, CDR2 and CDR3 domains of the light chain variable region having the sequence set forth in SEQ ID NO:8, for administration:
(a) once on Day 1 at a dose of 2400 mg, followed by a dose of 3000 mg at Week 2 and once every eight weeks thereafter and once on Day 183 at a dose of 900 mg, followed by a dose of 3000 mg on Day 197 and once every eight weeks thereafter, to a patient weighing ≥40 to <60 kg;
(b) once on Day 1 at a dose of 2700 mg, followed by a dose of 3900 mg at Week 2 and once every eight weeks thereafter and once on Day 183 at a dose of 900 mg, followed by a dose of 3900 mg on Day 197 and once every eight weeks thereafter, to a patient weighing ≥60 to <100 kg; or
(c) once on Day 1 at a dose of 3000 mg, followed by a dose of 5400 mg at Week 2 and once every eight weeks thereafter and once on Day 183 at a dose of 900 mg, followed by a dose of 5400 mg on Day 197 and once every eight weeks thereafter, to a patient weighing ≥100 kg.

In one embodiment, the antibody is determined to be safe, tolerable and sufficiently non-immunogenic after multiple IV doses for use in C5-mediated GN patients, including LN and/or IgAN patients.

Further provided are kits that include a pharmaceutical composition containing an anti-C5 antibody, or antigen binding fragment thereof, such as ravulizumab, and a pharmaceutically acceptable carrier, in a therapeutically effective amount adapted for use in the methods described herein, optionally together with background therapy. In one embodiment, the kit comprises: (a) a dose of an anti-C5 antibody or antigen binding fragment thereof, comprising CDR1, CDR2 and CDR3 domains of the heavy chain variable region having the sequence set forth in SEQ ID NO:12, and CDR1, CDR2 and CDR3 domains of the light chain variable region having the sequence set forth in SEQ ID NO:8; and (b) instructions for using the anti-C5 antibody or antigen binding fragment thereof in the methods described herein.

In one embodiment, the kit comprises a dose of an anti-C5 antibody or antigen binding fragment thereof, wherein the anti-C5 antibody, or antigen binding fragment thereof, is administered, optionally together with a background therapy for treating C5-mediated glomerulonephritis (GN) in a human patient:
(a) once on Day 1 at a dose of 2400 mg, followed by a dose of 3000 mg at Week 2 and once every eight weeks thereafter to a patient weighing ≥40 to <60 kg;
(b) once on Day 1 at a dose of 2700 mg, followed by a dose of 3900 mg at Week 2 and once every eight weeks thereafter to a patient weighing ≥60 to <100 kg; or
(c) once on Day 1 at a dose of 3000 mg, followed by a dose of 5400 mg at Week 2 and once every eight weeks thereafter to a patient weighing ≥100 kg.

In another embodiment, the kit comprises a dose of an anti-C5 antibody or antigen binding fragment thereof, wherein the anti-C5 antibody, or antigen binding fragment thereof, is administered, optionally together with a background therapy for treating C5-mediated glomerulonephritis (GN) in a human patient:
(a) once on Day 1 at a dose of 2400 mg, followed by a dose of 3000 mg at Week 2 and once every eight weeks thereafter and once on Day 183 at a dose of 900 mg, followed by a dose of 3000 mg on Day 197 and once every eight weeks thereafter, to a patient weighing ≥40 to <60 kg;
(b) once on Day 1 at a dose of 2700 mg, followed by a dose of 3900 mg at Week 2 and once every eight weeks thereafter and once on Day 183 at a dose of 900 mg, followed by a dose of 3900 mg on Day 197 and once every eight weeks thereafter, to a patient weighing ≥60 to <100 kg; or
(c) once on Day 1 at a dose of 3000 mg, followed by a dose of 5400 mg at Week 2 and once every eight weeks thereafter and once on Day 183 at a dose of 900 mg, followed by a dose of 5400 mg on Day 197 and once every eight weeks thereafter, to a patient weighing ≥100 kg.

In some embodiments, the disclosure relates to a composition, e.g., pharmaceutical composition or a medicament, comprising an effective amount of an anti-C5 antibody or an antigen binding fragment thereof, comprising heavy chain complementarity determining regions (HCDRs) comprising HCDR1, HCDR2 and HCDR3 sequences as set forth in SEQ ID NOs:19, 18 and 3, respectively, and light chain complementarity determining regions (LCDRs) comprising LCDR1, LCDR2 and LCDR3 sequences as set forth in SEQ ID NOs:4, and 6, respectively, for use in the treatment of C5-mediated GN, such as LN and/or IgAN, in a human patient, wherein the composition optionally comprises background therapy for treating the C5-mediated GN. Specifically, provided herein are compositions comprising effective amounts of ravulizumab (ULTOMIRIS®) or the antigen-binding fragment thereof, for treatment of C5-mediated GN, such as LN and/or IgAN, in a human patient. In some embodiments, the effective amount comprises use of the above dosages and scheduling of the anti-C5 antibody, e.g., ravulizumab.

In some embodiments, the disclosure relates to use of an effective amount of an anti-C5 antibody, or antigen binding fragment thereof, comprising heavy chain complementarity determining regions (HCDRs) comprising HCDR1, HCDR2 and HCDR3 sequences as set forth in SEQ ID NOs:19, 18 and 3, respectively, and light chain complementarity determining regions (LCDRs) comprising LCDR1, LCDR2 and LCDR3 sequences as set forth in SEQ ID NOs:4, 5 and 6, respectively, in the manufacture of a composition, e.g., pharmaceutical composition or a medicament, for treating C5-mediated GN, such as LN and/or IgAN, in a human patient, wherein the composition optionally comprises background therapy for the treatment of the C5-mediated GN. Specifically, provided herein are use of an effective amount of ravulizumab (ULTOMIRIS®) or the antigen-binding fragment thereof, in the manufacture of a composition, e.g., pharmaceutical composition or a medicament, for treating C5-mediated GN, such as LN and/or IgAN, in a human patient, wherein the composition comprises background therapy for the treatment of the C5-mediated GN. In some embodiments, the effective amount comprises use of the above dosages and scheduling of the anti-C5 antibody, e.g., ravulizumab, optionally together with dosages and scheduling of the background therapy. In some embodiments, the optional background therapy comprises (a) background therapy for treating LN comprising an immunosuppressant, e.g., a corticosteroid and/or mycophenolate mofetil or (b) background therapy for treating IgAN comprising renin-angiotensin system (RAS) inhibitor, e.g., an angiotensin-converting enzyme (ACE) inhibitor or a angiotensin II receptor blocker (ARB).

### BRIEF DESCRIPTION OF THE DRAWINGS

FIG. 1 is a schematic depicting the overall study design for the LN cohort. Randomization is stratified by whether corticosteroid induction treatment was initiated prior to Screening versus during the Screening Period. Background therapy consists of corticosteroids and mycophenolate mofetil. Weight based dosing regimen (see Example 1) are based on the last recorded study visit body weight. Abbreviations: LN=lupus nephritis; PK=pharmacokinetics.

FIG. 2 is a schematic depicting the overall study design for the IgAN cohort. Randomization is stratified by mean proteinuria (1 to 2 g/day versus >2 g/day) based on 2 valid 24-hr urine collections during the Screening Period. Background therapy consisting of stable maximally tolerated dose of ACE inhibitors or ARBs. Weight based dosing regimen (see Example 1) is based on the last recorded study visit body weight. Abbreviations: ACE=angiotensin-converting enzyme; ARB=angiotensin II receptor blocker; IgAN=immunoglobulin A nephropathy; PK=pharmacokinetics.

### DETAILED DESCRIPTION

### I. Definitions

As used herein, the term “subject” or “patient” is a human patient (e.g., a patient having hematopoietic LN and/or IgAN.

As used herein, the term “pediatric” patient is a human patient that has been classified by a physician or caretaker as belonging to a non-adult category and can include, e.g., newborn (both preterm and of term), infants, children, and adolescents. Typically, pediatric patients are patients under 18 years of age (<18 years of age).

As used herein, the term “adult” patient is a human patient that has been classified by a physician or caretaker as such, e.g., one who is not a newborn, infant, child or adolescent, e.g., based on age, developmental status, physiological features, etc. Typically, adult patients are patients who are 18 years of age or older (≥18 years of age).

As used herein, the term Glomerulonephritis (GN) refers to a group of renal diseases affecting the glomeruli, e.g., due to damage mediated by immunological mechanisms. A large proportion of the disease manifestations are caused by disturbances in the complement system. As used herein, C5-mediated glomerular nephritis (GN) refers to GN caused, in whole or part, due to complement component C5. C5-mediated GN includes disorders, such as lupus nephritis (LN) and immunoglobulin A nephropathy (IgAN).

As used herein, Lupus Nephritis (LN) refers to inflammation of the kidney that represents a serious progression of systemic lupus erythematosus (SLE). Symptoms of LN include, but are not limited to foamy urine (due to proteinuria, excess protein in urine), edema (e.g., in the hands, ankles or feet), high blood pressure (hypertension), kidney inflammation, kidney impairment, joint pain or swelling, muscle pain, fever with no known cause, high levels of creatinine in the blood, and/or a red rash (e.g., often on the face, across the nose and cheeks, sometimes called a butterfly rash because of its shape).

LN occurs in approximately 50% of patients with SLE, an autoimmune disorder caused by loss of tolerance to self-antigens, the production of autoantibodies, and deposition of complement-fixing immune complexes (ICs) in injured tissues (see, e.g., Bao et al., Kidney Dis. 2015; 1(2):91-99). The diagnosis of LN is determined by kidney biopsy according to the 2018 International Society of Nephrology/Renal Pathology Society (ISN/RPS) nomenclature and classification revised from the 2003 report (see, e.g., Bajema et al., Kidney Int. 2018; 93(4):789-796) and Markowitz et al., Kidney Int 2007; 71(6):491-495). In total there are 6 classes of LN: Classes I to VI (Markowitz, 2007). The subset of patients with SLE that develop LN have the worst prognosis (see, e.g., Hoover et al., Kidney Int. 2016; 90(3):487-492). Lupus nephritis leading to CKD is an independent major risk factor for overall mortality and morbidity attributed to cardiovascular disease and septic shock. With current induction and maintenance therapies, the 5 year mortality is approximately 20% and the risk of developing ESRD at 5, 10, and 15 years are 11%, 17%, and 22%, respectively (see, e.g., Mageau et al., Autoimmun Rev. 2019; 18(7):733-737). Recurrence of LN after treatment (renal flare) occurs within 1 year in up to 25% of patients and is associated with increased risk of CKD progression (see, e.g., Almaani, Clin J Am Soc Nephrol. 2017; 12(5):825-835).

The pathophysiology of LN involves multiple overlapping pathways where complement serves as a mediator of an abnormal immune response (see, e.g., Bao et al, 2015; Pickering et al., Rheumatology (Oxford). 2000; 39(2):133-141; and Schur et al., Nephrologie. 1988; 9(2):53-60). The terminal complement components (C5a and terminal complement complex [C5b-9]) trigger acute cellular inflammatory responses through activation of interleukin and cytokine signaling. Complement also serves to fix immunoglobulins and ICs in the kidney. In fact, complement and complement split products are a prominent histologic finding in kidney biopsies of LN (see, e.g., Biesecker et al., J Exp Med. 1981; 154(6):1779-1794, 1981 and Wilson et al., Kidney Int. 2019; 95(3):655-665). Serum levels of these autoimmune and complement biomarkers are linked with disease activity (see, e.g., Birmingham et al., Semin Nephrol. 2015; 35(5):444-454 and Dall'Era et al., Arthritis Care Res. 2011; 63(3):351-357). Decreases in complement components 3, 4, and 1q (C3, C4, and C1q) are associated with de novo LN and LN flares. Likewise, levels of complement biomarkers correlate with disease activity in SLE (see, e.g., Kim et al, Arthritis Rheumatol. 2019; 71(3):420-430).

The American College of Rheumatology (ACR), and joint recommendations from the European League Against Rheumatism (EULAR) and European Renal Association-European Dialysis and Transplant Association (ERA-EDTA), recommend immunosuppression treatment for Class III, IV, III/V, and IVN LN also called “proliferative” LN (see, e.g., Bertsias et al., Ann Rheum Dis. 2012; 71:1771-1782). The guidelines agree on induction treatment with glucocorticoids plus mycophenolate mofetil (MMF) or cyclophosphamide. For maintenance therapy, the guidelines agree on MMF or azathioprine, with or without low dose glucocorticoids. In patients with LN, the main goal of therapy is prevention of CKD progression, ESRD, and death. Lack of achievement of remission, in particular complete remission, is one of the major risk factors for progression of renal disease. Hence, short term complete and partial renal remissions are used to assess the efficacy of standard of care and novel therapies. However, after 6 to 12 months of treatment, only 10% to 40% of patients achieve a Complete Renal Response (CRR) with standard of care (see, e.g., Parikh et al., J Am Soc Nephrol. 2016; 27(10):2929-2939).

As used herein, IgA nephropathy (IgAN), also known as Berger's disease, refers to the most common global primary glomerulonephropathy that can progress to renal failure (see, e.g., Lai et al., F1000Research. 2016; 5:161). Symptoms of IgAN include, but are not limited to, hematuria (blood in the urine that can sometimes make it pink, dark brown or cola colored), edema (e.g., in the hands, ankles or feet), pain on the side of the back (flank pain), high blood pressure (hypertension), and/or foamy urine (due to proteinuria, excess protein in urine).

Immunoglobulin A (IgA) nephropathy is a lifelong disease leading to CKD and progresses to ESRD in 30% to 40% of patients over the course of 20 to 30 years (Lai, 2016). Patients initially present with hematuria and hypertension and proteinuria develops as the disease progresses. Diagnosis of IgAN is made by renal biopsy demonstrating IgA immunofluorescence in the glomeruli usually co-dominant with complement 3 (C3) according to the Oxford Classification nomenclature (see, e.g., KDIGO Clinical practice guideline for glomerulonephritis. Kidney International Supplements. 2012; 2(2):140, Rizk et al., Front Immunol. 2019; 10:504; and Trimarchi et al., Kidney Int 2017; 91(5):1014-1021).

The pathophysiology of IgAN is related to the overproduction of under-glycosylated immunoglobulin A1 (IgA1) which accumulates in the kidney glomeruli. However, aberrant galactosylation alone is insufficient to induce renal injury; glycan-specific immunoglobulin A (IgA) and immunoglobulin G (IgG) autoantibodies that recognize the under-galactosylated IgA1 molecule likely also contribute. This process leads to the local inflammation and complement activation in the kidney (see, e.g., Oortwijn et al., Semin Nephrol. 2008; 28(1):58-65). Both the alternative and lectin complement pathways may be activated, leading to generation of anaphylatoxins, and the membrane attack complex terminal complement (C5b-9), with subsequent promotion of inflammatory mediators (see, e.g., Maillard et al., J Am Soc Nephrol. 2015; 26(7):1503-1512). Complement component 4 (C4) and C3 complexes and activated C3 products are elevated in up to 30% of patients with IgAN. Activated C3 products are associated with elevated levels of proteinuria and hematuria compared to patients with IgAN who have normal levels, and correlate with deterioration of renal function (see, e.g., Zwirner et al., Kidney Int. 1997; 51(4):1257-64). Complement activity on kidney biopsy and circulating complement proteins are associated with disease activity and progression of CKD. Together these findings suggest a role of complement in the pathophysiology and the prognostic value of complement biomarkers in IgAN (see, e.g., Rizk et al., Front Immunol. 2019; 10:504).

Treatments for IgAN include RAS blocking agents, such as angiotensin-converting enzyme (ACE) inhibitors or angiotensin II receptor blockers (ARBs). These therapies are aimed at controlling blood pressure, preserving kidney function through decreasing intraglomerular pressure which in turn reduces proteinuria, and suppressing the immune response. These treatments are insufficient in preserving renal function as the proportions of patients who progress to CKD and ESRD are high. Patients with baseline hypertension and proteinuria>1 g/day are at increased risk for progression (see Reich, et al., J Am Soc Nephrol. 2007; 18(12):3177-3183.).

As used herein, “effective treatment” refers to treatment producing a beneficial effect, e.g., amelioration of at least one symptom of a disease or disorder. A beneficial effect can take the form of an improvement over baseline, e.g., an improvement over a measurement or observation made prior to initiation of therapy according to the method. Effective treatment may refer to alleviation of at least one symptom of LN (e.g., foamy urine (due to proteinuria, excess protein in urine), edema (e.g., in the hands, ankles or feet), high blood pressure (hypertension), kidney inflammation, kidney impairment, joint pain or swelling, muscle pain, fever with no known cause, high levels of creatinine in the blood, and/or a red rash (e.g., often on the face, across the nose and cheeks, sometimes called a butterfly rash because of its shape). Effective treatment may refer to alleviation of at least one symptom of IgAN (e.g., hematuria (blood in the urine that can sometimes make it pink, dark brown or cola colored), edema (e.g., in the hands, ankles or feet), pain on the side of the back (flank pain), high blood pressure (hypertension), and/or foamy urine (due to proteinuria, excess protein in urine)).

The term “effective amount” refers to an amount of an agent that provides the desired biological, therapeutic and/or prophylactic result. That result can be reduction, amelioration, palliation, lessening, delaying and/or alleviation of one or more of the signs, symptoms or causes of a disease, or any other desired alteration of a biological system. In one example, an “effective amount” is the amount of anti-C5 antibody, or antigen binding fragment thereof, clinically proven to alleviate at least one symptom of LN (e.g., foamy urine (due to proteinuria, excess protein in urine), edema (e.g., in the hands, ankles or feet), high blood pressure (hypertension), kidney inflammation, kidney impairment, joint pain or swelling, muscle pain, fever with no known cause, high levels of creatinine in the blood, and/or a red rash (e.g., often on the face, across the nose and cheeks, sometimes called a butterfly rash because of its shape)) and/or IgAN (e.g., hematuria (blood in the urine that can sometimes make it pink, dark brown or cola colored), edema (e.g., in the hands, ankles or feet), pain on the side of the back (flank pain), high blood pressure (hypertension), and/or foamy urine (due to proteinuria, excess protein in urine)).

An effective amount can be administered in one or more administrations.

As used herein, the term “loading dose” refers to the first dose administered (e.g., during an administration cycle).

As used herein, the terms “maintenance” and “maintenance phase” are used interchangeably and refer to the second phase of treatment. In certain embodiments, treatment is continued as long as clinical benefit is observed or until unmanageable toxicity or disease progression occurs.

As used herein, the term “serum trough level” refers to the lowest level that the agent (e.g., the anti-C5 antibody, or antigen binding fragment thereof) or medicine is present in the serum. In contrast, a “peak serum level,” refers to the highest level of the agent in the serum. The “average serum level,” refers to the mean level of the agent in the serum over time.

The term “antibody” describes a polypeptide comprising at least one antibody-derived antigen binding site (e.g., VH/VL region or F_{V}, or CDR). Antibodies include known forms of antibodies, e.g., the antibody can be a human antibody, a humanized antibody, a bispecific antibody or a chimeric antibody. The antibody also can be a Fab, Fab′2, ScFv, SMIP, Affibody®, nanobody or a single-domain antibody. The antibody also can be of any of the following isotypes: IgG1, IgG2, IgG3, IgG4, IgM, IgA1, IgA2, IgAsec, IgD, IgE or combinations thereof. The antibody can be a naturally occurring antibody or an antibody that has been altered by a protein engineering technique (e.g., by mutation, deletion, substitution, conjugation to a non-antibody moiety). An antibody can include, for example, one or more variant amino acids (compared to a naturally occurring antibody) that change a property (e.g., a functional property) of the antibody. Numerous such alterations are known in the art that affect, e.g., half-life, effector function, and/or immune responses to the antibody in a patient. The term antibody also includes artificial or engineered polypeptide constructs that comprise at least one antibody-derived antigen binding site.

### II. Anti-C5 Antibodies

Anti-C5 antibodies described herein bind to complement component C5 (e.g., human C5) and inhibit the cleavage of C5 into fragments C5a and C5b. As described above, such antibodies also have, for example, improved pharmacokinetic properties relative to other anti-C5 antibodies (e.g., eculizumab) used for therapeutic purposes.

Anti-C5 antibodies (or VH/VL domains derived therefrom) suitable for use in the methods described herein can be generated using methods known in the art. Alternatively, art recognized anti-C5 antibodies can be used. Antibodies that compete for binding to C5 with any of these art recognized antibodies or antibodies described herein can also be used.

An exemplary anti-C5 antibody is ravulizumab comprising heavy and light chains having the sequences shown in SEQ ID NOs:14 and 11, respectively, or antigen binding fragments and variants thereof. Ravulizumab (also known as ULTOMIRIS®, BNJ441 and ALXN1210) is described in WO2015134894 and U.S. Pat. No. 9,079,949, the entire teachings of which are hereby incorporated by reference. The terms ravulizumab, BNJ441, and ALXN1210 may be used interchangeably throughout this document, but all refer to the same antibody. Ravulizumab selectively binds to human complement protein C5, inhibiting its cleavage to C5a and C5b during complement activation. This inhibition prevents the release of the proinflammatory mediator C5a and the formation of the cytolytic pore-forming membrane attack complex (MAC) C5b-9 while preserving the proximal or early components of complement activation (e.g., C3 and C3b) essential for the opsonization of microorganisms and clearance of immune complexes.

In other embodiments, the antibody comprises the heavy and light chain CDRs or variable regions of ravulizumab. Accordingly, in one embodiment, the antibody comprises the CDR1, CDR2 and CDR3 domains of the VH region of ravulizumab having the sequence set forth in SEQ ID NO:12, and the CDR1, CDR2 and CDR3 domains of the VL region of ravulizumab having the sequence set forth in SEQ ID NO:8. In another embodiment, the antibody comprises heavy chain CDR1, CDR2 and CDR3 domains having the sequences set forth in SEQ ID NOs:19, 18 and 3, respectively, and light chain CDR1, CDR2 and CDR3 domains having the sequences set forth in SEQ ID NOs:4, 5 and 6, respectively. In another embodiment, the antibody comprises VH and VL regions having the amino acid sequences set forth in SEQ ID NO:12 and SEQ ID NO:8, respectively.

Another exemplary anti-C5 antibody is antibody BNJ421 comprising heavy and light chains having the sequences shown in SEQ ID NOs:20 and 11, respectively, or antigen binding fragments and variants thereof. BNJ421 (also known as ALXN1211) is described in WO2015134894 and U.S. Pat. No. 9,079,949, the entire teachings of which are hereby incorporated by reference.

In other embodiments, the antibody comprises the heavy and light chain CDRs or variable regions of BNJ421. Accordingly, in one embodiment, the antibody comprises the CDR1, CDR2 and CDR3 domains of the VH region of BNJ421 having the sequence set forth in SEQ ID NO:12, and the CDR1, CDR2 and CDR3 domains of the VL region of BNJ421 having the sequence set forth in SEQ ID NO:8. In another embodiment, the antibody comprises heavy chain CDR1, CDR2 and CDR3 domains having the sequences set forth in SEQ ID NOs:19, 18 and 3, respectively, and light chain CDR1, CDR2 and CDR3 domains having the sequences set forth in SEQ ID NOs:4, 5 and 6, respectively. In another embodiment, the antibody comprises VH and VL regions having the amino acid sequences set forth in SEQ ID NO:12 and SEQ ID NO:8, respectively.

The exact boundaries of CDRs are defined differently according to different methods. In some embodiments, the positions of the CDRs or framework regions within a light or heavy chain variable domain are as defined by Kabat et al. [(1991) “Sequences of Proteins of Immunological Interest.” NIH Publication No. 91-3242, U.S. Department of Health and Human Services, Bethesda, MD]. In such cases, the CDRs can be referred to as “Kabat CDRs” (e.g., “Kabat LCDR2” or “Kabat HCDR1”). In some embodiments, the positions of the CDRs of a light or heavy chain variable region are as defined by Chothia et al. (Nature, 342:877-83, 1989). Accordingly, these regions can be referred to as “Chothia CDRs” (e.g., “Chothia LCDR2” or “Chothia HCDR3”). In some embodiments, the positions of the CDRs of the light and heavy chain variable regions can be defined by a Kabat-Chothia combined definition. In such embodiments, these regions can be referred to as “combined Kabat-Chothia CDRs.” Thomas, C. et al. (Mol. Immunol., 33:1389-401, 1996) exemplifies the identification of CDR boundaries according to Kabat and Chothia numbering schemes.

Another exemplary anti-C5 antibody is the 7086 antibody described in U.S. Pat. Nos. 8,241,628 and 8,883,158. In one embodiment, the antibody comprises the heavy and light chain CDRs or variable regions of the 7086 antibody (see U.S. Pat. Nos. 8,241,628 and 8,883,158). In another embodiment, the antibody, or antigen binding fragment thereof, comprises heavy chain CDR1, CDR2 and CDR3 domains having the sequences set forth in SEQ ID NOs:21, 22 and 23, respectively, and light chain CDR1, CDR2 and CDR3 domains having the sequences set forth in SEQ ID NOs:24, 25 and 26, respectively. In another embodiment, the antibody, or antigen binding fragment thereof, comprises the VH region of the 7086 antibody having the sequence set forth in SEQ ID NO:27, and the VL region of the 7086 antibody having the sequence set forth in SEQ ID NO:28.

Another exemplary anti-C5 antibody is the 8110 antibody also described in U.S. Pat. Nos. 8,241,628 and 8,883,158. In one embodiment, the antibody comprises the heavy and light chain CDRs or variable regions of the 8110 antibody. In another embodiment, the antibody, or antigen binding fragment thereof, comprises heavy chain CDR1, CDR2 and CDR3 domains having the sequences set forth in SEQ ID NOs:29, 30 and 31, respectively, and light chain CDR1, CDR2 and CDR3 domains having the sequences set forth in SEQ ID NOs:32, 33 and 34, respectively. In another embodiment, the antibody comprises the VH region of the 8110 antibody having the sequence set forth in SEQ ID NO:35, and the VL region of the 8110 antibody having the sequence set forth in SEQ ID NO:36.

Another exemplary anti-C5 antibody is the 305L05 antibody described in U.S. Pat. No. 9,765,135. In one embodiment, the antibody comprises the heavy and light chain CDRs or variable regions of the 305L05 antibody. In another embodiment, the antibody, or antigen binding fragment thereof, comprises heavy chain CDR1, CDR2 and CDR3 domains having the sequences set forth in SEQ ID NOs:37, 38 and 39, respectively, and light chain CDR1, CDR2 and CDR3 domains having the sequences set forth in SEQ ID NOs:40, 41 and 42, respectively. In another embodiment, the antibody comprises the VH region of the 305LO5 antibody having the sequence set forth in SEQ ID NO:43, and the VL region of the 305LO5 antibody having the sequence set forth in SEQ ID NO:44.

Another exemplary anti-C5 antibody is the SKY59 antibody (Fukuzawa, T. et al., Sci. Rep., 7:1080, 2017). In one embodiment, the antibody comprises the heavy and light chain CDRs or variable regions of the SKY59 antibody. In another embodiment, the antibody, or antigen binding fragment thereof, comprises a heavy chain comprising SEQ ID NO:45 and a light chain comprising SEQ ID NO:46.

In some embodiments, the anti-C5 antibody comprises the heavy and light chain variable regions or heavy and light chains of the REGN3918 antibody (see U.S. Pat. No. 10,633,434). In some embodiments, the anti-C5 antibody, or antigen-binding fragment thereof, comprises a heavy chain variable region sequence set forth in SEQ ID NO: 47 and a light chain variable region comprising the sequence set forth in SEQ ID NO: 48. In some embodiments, the anti-C5 antibody, or antigen-binding fragment thereof, comprises a heavy chain sequence set forth in SEQ ID NO: 49 and a light chain sequence set forth in SEQ ID NO: 50.

In some embodiments, an anti-C5 antibody described herein comprises a heavy chain CDR1 comprising, or consisting of, the following amino acid sequence: GHIFSNYWIQ (SEQ ID NO:19). In some embodiments, an anti-C5 antibody described herein comprises a heavy chain CDR2 comprising, or consisting of, the following amino acid sequence: EILPGSGHTEYTENFKD (SEQ ID NO:18). In some embodiments, an anti-C5 antibody described herein comprises a heavy chain variable region comprising the following amino acid sequence:
QVQLVQSGAE VKKPGASVKV SCKASGHIFS NYWIQWVRQA PGQGLEWMGE ILPGSGHTEY TENFKDRVTM TRDTSTSTVY MELSSLRSED TAVYYCARYF FGSSPNWYFD VWGQGTLVTV SS (SEQ ID NO:12).

In some embodiments, an anti-C5 antibody described herein comprises a light chain variable region comprising the following amino acid sequence:
DIQMTQSPSS LSASVGDRVT ITCGASENIY GALNWYQQKP GKAPKLLIYG ATNLADGVPS RFSGSGSGTD FTLTISSLQP EDFATYYCQN VLNTPLTFGQ GTKVEIK (SEQ ID NO:8).

An anti-C5 antibody described herein can, in some embodiments, comprise a variant human Fc constant region that binds to human neonatal Fc receptor (FcRn) with greater affinity than that of the native human Fc constant region from which the variant human Fc constant region was derived. The Fc constant region can, for example, comprise one or more (e.g., two, three, four, five, six, seven, or eight or more) amino acid substitutions relative to the native human Fc constant region from which the variant human Fc constant region was derived. The substitutions can increase the binding affinity of an IgG antibody containing the variant Fc constant region to FcRn at pH 6.0, while maintaining the pH dependence of the interaction. Methods for testing whether one or more substitutions in the Fc constant region of an antibody increase the affinity of the Fc constant region for FcRn at pH 6.0 (while maintaining pH dependence of the interaction) are known in the art and exemplified in the working examples. See, e.g., WO2015134894 and U.S. Pat. No. 9,079,949 the disclosures of each of which are incorporated herein by reference in their entirety.

Substitutions that enhance the binding affinity of an antibody Fc constant region for FcRn are known in the art and include, e.g., (1) the M252Y/S254T/T256E triple substitution (Dall'Acqua, W. et al., J. Biol. Chem., 281:23514-24, 2006); (2) the M428L or T250Q/M428L substitutions (Hinton, P. et al., J. Biol. Chem., 279:6213-6, 2004; Hinton, P. et al., J. Immunol., 176:346-56, 2006); and (3) the N434A or T307/E380A/N434A substitutions (Petkova, S. et al., Int. Immunol., 18:1759-69, 2006). The additional substitution pairings: P257I/Q311I, P257I/N434H and D376V/N434H (Datta-Mannan, A. et al., J. Biol. Chem., 282:1709-17, 2007), the disclosures of each of which are incorporated herein by reference in their entirety.

In some embodiments, the variant constant region has a substitution at EU amino acid position 255 for valine. In some embodiments, the variant constant region has a substitution at EU amino acid position 309 for asparagine. In some embodiments, the variant constant region has a substitution at EU amino acid position 312 for isoleucine. In some embodiments, the variant constant region has a substitution at EU amino acid position 386.

In some embodiments, the variant Fc constant region comprises no more than 30 (e.g., no more than 29, 28, 27, 26, 25, 24, 23, 22, 21, 20, 19, 18, 17, 16, 15, 14, 13, 12, 11, 10, 9, 8, 7, 6, 5, 4, 3 or 2) amino acid substitutions, insertions, or deletions relative to the native constant region from which it was derived. In some embodiments, the variant Fc constant region comprises one or more amino acid substitutions selected from the group consisting of: M252Y, S254T, T256E, N434S, M428L, V259I, T250I and V308F. In some embodiments, the variant human Fc constant region comprises a methionine at position 428 and an asparagine at position 434 of a native human IgG Fc constant region, each in EU numbering. In some embodiments, the variant Fc constant region comprises a 428L/434S double substitution as described in, e.g., U.S. Pat. No. 8,088,376.

In some embodiments the precise location of these mutations may be shifted from the native human Fc constant region position due to antibody engineering. For example, the 428L/434S double substitution when used in a IgG2/4 chimeric Fc may correspond to 429L and 435S as in the M429L and N435S variants found in ravulizumab and described in U.S. Pat. No. 9,079,949 the disclosure of which is incorporated herein by reference in its entirety.

In some embodiments, the variant constant region comprises a substitution at amino acid position 237, 238, 239, 248, 250, 252, 254, 255, 256, 257, 258, 265, 270, 286, 289, 297, 298, 303, 305, 307, 308, 309, 311, 312, 314, 315, 317, 325, 332, 334, 360, 376, 380, 382, 384, 385, 386, 387, 389, 424, 428, 433, 434 or 436 (EU numbering) relative to the native human Fc constant region. In some embodiments, the substitution is selected from the group consisting of: methionine for glycine at position 237; alanine for proline at position 238; lysine for serine at position 239; isoleucine for lysine at position 248; alanine, phenylalanine, isoleucine, methionine, glutamine, serine, valine, tryptophan, or tyrosine for threonine at position 250; phenylalanine, tryptophan, or tyrosine for methionine at position 252; threonine for serine at position 254; glutamic acid for arginine at position 255; aspartic acid, glutamic acid, or glutamine for threonine at position 256; alanine, glycine, isoleucine, leucine, methionine, asparagine, serine, threonine, or valine for proline at position 257; histidine for glutamic acid at position 258; alanine for aspartic acid at position 265; phenylalanine for aspartic acid at position 270; alanine, or glutamic acid for asparagine at position 286; histidine for threonine at position 289; alanine for asparagine at position 297; glycine for serine at position 298; alanine for valine at position 303; alanine for valine at position 305; alanine, aspartic acid, phenylalanine, glycine, histidine, isoleucine, lysine, leucine, methionine, asparagine, proline, glutamine, arginine, serine, valine, tryptophan, or tyrosine for threonine at position 307; alanine, phenylalanine, isoleucine, leucine, methionine, proline, glutamine, or threonine for valine at position 308; alanine, aspartic acid, glutamic acid, proline, or arginine for leucine or valine at position 309; alanine, histidine, or isoleucine for glutamine at position 311; alanine or histidine for aspartic acid at position 312; lysine or arginine for leucine at position 314; alanine or histidine for asparagine at position 315; alanine for lysine at position 317; glycine for asparagine at position 325; valine for isoleucine at position 332; leucine for lysine at position 334; histidine for lysine at position 360; alanine for aspartic acid at position 376; alanine for glutamic acid at position 380; alanine for glutamic acid at position 382; alanine for asparagine or serine at position 384; aspartic acid or histidine for glycine at position 385; proline for glutamine at position 386; glutamic acid for proline at position 387; alanine or serine for asparagine at position 389; alanine for serine at position 424; alanine, aspartic acid, phenylalanine, glycine, histidine, isoleucine, lysine, leucine, asparagine, proline, glutamine, serine, threonine, valine, tryptophan, or tyrosine for methionine at position 428; lysine for histidine at position 433; alanine, phenylalanine, histidine, serine, tryptophan, or tyrosine for asparagine at position 434; and histidine for tyrosine or phenylalanine at position 436, all in EU numbering.

Suitable anti-C5 antibodies for use in the methods described herein, in some embodiments, comprise a heavy chain polypeptide comprising the amino acid sequence set forth in SEQ ID NO:14 and/or a light chain polypeptide comprising the amino acid sequence set forth in SEQ ID NO:11. Alternatively, the anti-C5 antibodies for use in the methods described herein, in some embodiments, comprise a heavy chain polypeptide comprising the amino acid sequence set forth in SEQ ID NO:20 and/or a light chain polypeptide comprising the amino acid sequence set forth in SEQ ID NO:11.

In one embodiment, the antibody binds to C5 at pH 7.4 and 25° C. (and, otherwise, under physiologic conditions) with an affinity dissociation constant (K_{D}) that is at least 0.1 (e.g., at least 0.15, 0.175, 0.2, 0.25, 0.275, 0.3, 0.325, 0.35, 0.375, 0.4, 0.425, 0.45, 0.475, 0.5, 0.525, 0.55, 0.575, 0.6, 0.625, 0.65, 0.675, 0.7, 0.725, 0.75, 0.775, 0.8, 0.825, 0.85, 0.875, 0.9, 0.925, 0.95, or 0.975) nM. In one embodiment, the antibody binds to C5 at pH 7.4 and 25° C. (and, otherwise, under physiologic conditions) with an affinity dissociation constant (K_{D}) that is about 0.5 nM. In some embodiments, the K_{D} of the anti-C5 antibody, or antigen binding fragment thereof, is no greater than 1 (e.g., no greater than 0.9, 0.8, 0.7, 0.6, 0.5, 0.4, 0.3, or 0.2) nM. In some embodiments, the antibody binds to C5 at pH 6.0 and 25° C. (and, otherwise, under physiologic conditions) with a K_{D} that is about 22 nM.

In other embodiments, the [(K_{D} of the antibody for C5 at pH 6.0 at 25° C.)/(K_{D} of the antibody for C5 at pH 7.4 at 25 C)] is greater than 21 (e.g., greater than 22, 23, 24, 25, 26, 27, 28, 29, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 350, 400, 450, 500, 600, 700, 800, 900, 1000, 1500, 2000, 2500, 3000, 3500, 4000, 4500, 5000, 5500, 6000, 6500, 7000, 7500 or 8000)

Methods for determining whether an antibody binds to a protein antigen and/or the affinity for an antibody to a protein antigen are known in the art. The binding of an antibody to a protein antigen, for example, can be detected and/or quantified using a variety of techniques such as, but not limited to, Western blot, dot blot, surface plasmon resonance (SPR) detection (e.g., BIAcore system; Pharmacia Biosensor AB, Uppsala, Sweden and Piscataway, N.J.), or enzyme-linked immunosorbent assay (ELISA; Benny K. C. Lo (2004) “Antibody Engineering: Methods and Protocols,” Humana Press (ISBN: 1588290921); Johne, B. et al., J. Immunol. Meth., 160:191-8, 1993; Jonsson, U. et al., Ann. Biol. Clin., 51:19-26, 1993; Jönsson, U. et al., Biotechniques, 11:620-7, 1991). In addition, methods for measuring the affinity (e.g., dissociation and association constants) are set forth in the working examples.

As used herein, the term “kₐ” refers to the rate constant for association of an antibody to an antigen. The term “k_{d}” refers to the rate constant for dissociation of an antibody from the antibody/antigen complex. And the term “K_{D}” refers to the equilibrium dissociation constant of an antibody-antigen interaction. The equilibrium dissociation constant is deduced from the ratio of the kinetic rate constants, K_{D}=kₐ/k_{d}. Such determinations can be measured, for example, at 25 C or 37 C (see the working examples). The kinetics of antibody binding to human C5 can be determined, for example, at pH 8.0, 7.4, 7.0, 6.5 and 6.0 via SPR on a BIAcore 3000 instrument using an anti-Fc capture method to immobilize the antibody.

In one embodiment, the anti-C5 antibody, or antigen binding fragment thereof, blocks the cleavage of C5 into C5a and C5b. Through this blocking effect, for example, the pro-inflammatory effects of C5a and the generation of the C5b-9 membrane attack complex (MAC) at the surface of a cell are inhibited.

Methods for determining whether a particular antibody described herein inhibits C5 cleavage are known in the art. Inhibition of human complement component C5 can reduce the cell-lysing ability of complement in a subject's body fluids. Such reductions of the cell-lysing ability of complement present in the body fluid(s) can be measured by methods known in the art such as, for example, by a conventional hemolytic assay such as the hemolysis assay (Kabat and Mayer (eds.), “Experimental Immunochemistry, 2^{nd} Edition,” 135-240, Springfield, IL, CC Thomas (1961), pages 135-139), or a conventional variation of that assay such as the chicken erythrocyte hemolysis method (Hillmen, P. et al., N. Engl. J. Med., 350:552-9, 2004). Methods for determining whether a candidate compound inhibits the cleavage of human C5 into forms C5a and C5b are known in the art (Evans, M. et al., Mol. Immunol., 32:1183-95, 1995). The concentration and/or physiologic activity of C5a and C5b in a body fluid can be measured, for example, by methods known in the art. For C5b, hemolytic assays or assays for soluble C5b-9 as discussed herein can be used. Other assays known in the art can also be used. Using assays of these or other suitable types, candidate agents capable of inhibiting human complement component C5 can be screened.

Immunological techniques such as, but not limited to, ELISA can be used to measure the protein concentration of C5 and/or its split products to determine the ability of an anti-C5 antibody, or antigen binding fragment thereof, to inhibit conversion of C5 into biologically active products. In some embodiments, C5a generation is measured. In some embodiments, C5b-9 neoepitope-specific antibodies are used to detect MAC formation.

Hemolytic assays can be used to determine the inhibitory activity of an anti-C5 antibody, or antigen binding fragment thereof, on complement activation. To determine the effect of an anti-C5 antibody, or antigen binding fragment thereof, on classical complement pathway-mediated hemolysis in a serum test solution in vitro, for example, sheep erythrocytes coated with hemolysin or chicken erythrocytes sensitized with anti-chicken erythrocyte antibody are used as target cells. The percentage of lysis is normalized by considering 100% lysis equal to the lysis occurring in the absence of the inhibitor. In some embodiments, the classical complement pathway is activated by a human IgM antibody, for example, as utilized in the Wieslab® Classical Pathway Complement Kit (Wieslab® COMPL CP310, Euro-Diagnostica, Sweden). Briefly, the test serum is incubated with an anti-C5 antibody, or antigen binding fragment thereof, in the presence of a human IgM antibody. The amount of C5b-9 that is generated is measured by contacting the mixture with an enzyme conjugated anti-C5b-9 antibody and a fluorogenic substrate and measuring the absorbance at the appropriate wavelength. As a control, the test serum is incubated in the absence of the anti-C5 antibody, or antigen binding fragment thereof. In some embodiments, the test serum is a C5-deficient serum reconstituted with a C5 polypeptide.

To determine the effect of an anti-C5 antibody, or antigen binding fragment thereof, on alternative pathway-mediated hemolysis, unsensitized rabbit or guinea pig erythrocytes can be used as the target cells. In some embodiments, the serum test solution is a C5-deficient serum reconstituted with a C5 polypeptide. The percentage of lysis is normalized by considering 100% lysis equal to the lysis occurring in the absence of the inhibitor. In some embodiments, the alternative complement pathway is activated by lipopolysaccharide molecules, for example, as utilized in the Wieslab® Alternative Pathway Complement Kit (Wieslab® COMPL AP330, Euro-Diagnostica, Sweden). Briefly, the test serum is incubated with an anti-C5 antibody, or antigen binding fragment thereof, in the presence of lipopolysaccharide. The amount of C5b-9 that is generated is measured by contacting the mixture with an enzyme conjugated anti-C5b-9 antibody and a fluorogenic substrate and measuring the fluorescence at the appropriate wavelength. As a control, the test serum is incubated in the absence of the anti-C5 antibody, or antigen binding fragment thereof.

In some embodiments, C5 activity, or inhibition thereof, is quantified using a CH50 eq assay. The CH50 eq assay is a method for measuring the total classical complement activity in serum. This test is a lytic assay, which uses antibody-sensitized erythrocytes as the activator of the classical complement pathway and various dilutions of the test serum to determine the amount required to give 50% lysis (CH50). The percent hemolysis can be determined, for example, using a spectrophotometer. The CH50 eq assay provides an indirect measure of terminal complement complex (TCC) formation, since the TCC themselves are directly responsible for the hemolysis that is measured. The assay is known and commonly practiced by those of skill in the art. Briefly, to activate the classical complement pathway, undiluted serum samples (e.g., reconstituted human serum samples) are added to microassay wells containing the antibody-sensitized erythrocytes to thereby generate TCC. Next, the activated sera are diluted in microassay wells, which are coated with a capture reagent (e.g., an antibody that binds to one or more components of the TCC). The TCC present in the activated samples bind to the monoclonal antibodies coating the surface of the microassay wells. The wells are washed and to each well is added a detection reagent that is detectably labeled and recognizes the bound TCC. The detectable label can be, e.g., a fluorescent label or an enzymatic label. The assay results are expressed in CH50 unit equivalents per milliliter (CH50 U Eq/mL).

Inhibition, e.g., as it pertains to terminal complement activity, includes at least a 5 (e.g., at least a 6, 7, 8, 9, 10, 15, 20, 25, 30, 35, 40, 45, 50, 55 or 60) % decrease in the activity of terminal complement in, e.g., a hemolytic assay or CH50 eq assay as compared to the effect of a control antibody (or antigen-binding fragment thereof) under similar conditions and at an equimolar concentration. Substantial inhibition, as used herein, refers to inhibition of a given activity (e.g., terminal complement activity) of at least 40 (e.g., at least 45, 50, 55, 60, 65, 70, 75, 80, 85, 90, or 95 or greater) %. In some embodiments, an anti-C5 antibody described herein contains one or more amino acid substitutions relative to the CDRs of eculizumab (i.e., SEQ ID NOs:1-6), yet retains at least 30 (e.g., at least 31, 32, 33, 34, 35, 36, 37, 38, 39, 40, 41, 42, 43, 44, 45, 46, 47, 48, 49, 50, 55, 60, 65, 70, 75, 80, 85, 90 or 95) % of the complement inhibitory activity of eculizumab in a hemolytic assay or CH50 eq assay.

An anti-C5 antibody described herein has a serum half-life in humans that is at least 20 (e.g., at least 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54 or 55) days. In another embodiment, the anti-C5 antibody described herein has a serum half-life in humans that is at least 40 days. In another embodiment, the anti-C5 antibody described herein has a serum half-life in humans that is approximately 43 days. In another embodiment, the anti-C5 antibody described herein has a serum half-life in humans that is between 39-48 days. Methods for measuring the serum half-life of an antibody are known in the art. In some embodiments, an anti-C5 antibody, or antigen binding fragment thereof, described herein has a serum half-life that is at least 20 (e.g., at least 30, 35, 40, 45, 50, 55, 60, 65, 70, 75, 80, 85, 90, 95, 100, 125, 150, 175, 200, 250, 300, 400 or 500) % greater than the serum half-life of eculizumab, e.g., as measured in one of the mouse model systems described in the working examples (e.g., the C5-deficient/NOD/scid mouse or hFcRn transgenic mouse model system).

In one embodiment, the antibody competes for binding with, and/or binds to the same epitope on C5 as an antibody described herein. The term “binds to the same epitope” with reference to two or more antibodies means that the antibodies bind to the same segment of amino acid residues, as determined by a given method. Techniques for determining whether antibodies bind to the same epitope on C5 with an antibody described herein include, for example, epitope mapping methods, such as, x-ray analyses of crystals of antigen:antibody complexes, and hydrogen/deuterium exchange mass spectrometry (HDX-MS). Other methods monitor the binding of the antibody to peptide antigen fragments or mutated variations of the antigen where loss of binding due to a modification of an amino acid residue within the antigen sequence is often considered an indication of an epitope component. In addition, computational combinatorial methods for epitope mapping can also be used. These methods rely on the ability of the antibody of interest to affinity isolate specific short peptides from combinatorial phage display peptide libraries. Antibodies having the same VH and VL or the same CDR1, CDR2 and CDR3 sequences are expected to bind to the same epitope.

Antibodies that “compete with another antibody for binding to a target” refer to antibodies that inhibit (partially or completely) the binding of the other antibody to the target. Whether two antibodies compete with each other for binding to a target, i.e., whether and to what extent one antibody inhibits the binding of the other antibody to a target, may be determined using known competition experiments. In certain embodiments, an antibody competes with, and inhibits binding of another antibody to a target by at least 10%, 20%, 30%, 40%, 50%, 60%, 70%, 80%, 90% or 100%. The level of inhibition or competition may be different depending on which antibody is the “blocking antibody” (i.e., the antibody that is incubated first with the target). Competing antibodies can bind to, for example, the same epitope, an overlapping epitope or to adjacent epitopes (e.g., as evidenced by steric hindrance).

Anti-C5 antibodies, or antigen-binding fragments thereof described herein, used in the methods described herein can be generated using a variety of art-recognized techniques. Monoclonal antibodies can be obtained by various techniques familiar to those skilled in the art. Briefly, spleen cells from an animal immunized with a desired antigen are immortalized, commonly by fusion with a myeloma cell (Kohler, G. & Milstein, C., Eur. J. Immunol., 6:511-9, 1976)). Methods of immortalization include transformation with Epstein Barr Virus, oncogenes, or retroviruses or other methods known in the art. Colonies arising from single immortalized cells are screened for production of antibodies of the desired specificity and affinity for the antigen, and yield of the monoclonal antibodies produced by such cells may be enhanced by various techniques, including injection into the peritoneal cavity of a vertebrate host. Alternatively, one may isolate DNA sequences that encode a monoclonal antibody or a binding fragment thereof by screening a DNA library from human B cells (Huse, W. et al., Science, 246:1275-81, 1989).

In some embodiments, the anti-C5 antibody does not comprise eculizumab (SOLIRIS®) or an antigen-binding fragment thereof (e.g., comprising heavy and light chain complementarity determining regions (HCDR₁₋₃ and LCDR₁₋₃, respectively) of eculizumab). In some embodiments, the anti-C5 antibody is not a biosimilar of eculizumab (SOLIRIS®), e.g., ABP 959 antibody (manufactured by Amgen Inc., USA), ELIZARIA® (manufactured by Generium JNC, Russia), or SB12 (manufactured by Samsung Bioepis, Incheon, South Korea).

### III. Compositions

Also provided herein are compositions comprising an anti-C5 antibody, or antigen binding fragment thereof. In one embodiment, the composition comprises an anti-C5 antibody comprising the CDR1, CDR2 and CDR3 domains in a heavy chain variable region having the sequence set forth in SEQ ID NO:12, and the CDR1, CDR2 and CDR3 domains in a light chain variable region having the sequence set forth in SEQ ID NO:8. In another embodiment, the anti-C5 antibody comprises heavy and light chains having the sequences shown in SEQ ID NOs:14 and 11, respectively. In another embodiment, the anti-C5 antibody comprises heavy and light chains having the sequences shown in SEQ ID NOs:20 and 11, respectively.

The compositions can be formulated as a pharmaceutical solution, e.g., for administration to a subject for the treatment of C5-mediated GN, including LN and/or IgAN. The pharmaceutical compositions generally include a pharmaceutically acceptable carrier. As used herein, a “pharmaceutically acceptable carrier” refers to, and includes, any and all solvents, dispersion media, coatings, antibacterial and antifungal agents, isotonic and absorption delaying agents, and the like that are physiologically compatible. The compositions can include a pharmaceutically acceptable salt, e.g., an acid addition salt or a base addition salt, sugars, carbohydrates, polyols and/or tonicity modifiers.

The compositions can be formulated according to standard methods. Pharmaceutical formulation is an established art (see, for example, Gennaro (2000) “Remington: The Science and Practice of Pharmacy,” 20^{th} Edition, Lippincott, Williams & Wilkins (ISBN: 0683306472); Ansel et al. (1999) “Pharmaceutical Dosage Forms and Drug Delivery Systems,” 7^{th} Edition, Lippincott Williams & Wilkins Publishers (ISBN: 0683305727); and Kibbe (2000) “Handbook of Pharmaceutical Excipients American Pharmaceutical Association,” 3^{rd} Edition (ISBN: 091733096X)). In some embodiments, a composition can be formulated, for example, as a buffered solution at a suitable concentration and suitable for storage at 2-8 C (e.g., 4° C.). In some embodiments, a composition can be formulated for storage at a temperature below OC (e.g., −20° C. or −80° C.). In some embodiments, the composition can be formulated for storage for up to 2 years (e.g., 1 month, 2 months, 3 months, 4 months, 5 months, 6 months, 7 months, 8 months, 9 months, 10 months, 11 months, 1 year, 1½ years or 2 years) at 2-8° C. (e.g., 4° C.). Thus, in some embodiments, the compositions described herein are stable in storage for at least 1 year at 2-8° C. (e.g., 4° C.). The pharmaceutical compositions can be in a variety of forms. These forms include, e.g., liquid, semi-solid and solid dosage forms, such as liquid solutions (e.g., injectable and infusible solutions), dispersions or suspensions, tablets, pills, powders, liposomes and suppositories. The preferred form depends, in part, on the intended mode of administration and therapeutic application. Compositions containing a composition intended for systemic or local delivery, for example, can be in the form of injectable or infusible solutions. Accordingly, the compositions can be formulated for administration by a parenteral mode (e.g., intravenous, subcutaneous, intraperitoneal, or intramuscular injection). “Parenteral administration,” “administered parenterally” and other grammatically equivalent phrases, as used herein, refer to modes of administration other than enteral and topical administration, usually by injection, and include, without limitation, intravenous, intranasal, intraocular, pulmonary, intramuscular, intraarterial, intrathecal, intracapsular, intraorbital, intracardiac, intradermal, intrapulmonary, intraperitoneal, transtracheal, subcutaneous, subcuticular, intraarticular, subcapsular, subarachnoid, intraspinal, epidural, intracerebral, intracranial, intracarotid and intrasternal injection and infusion.

In some embodiments, the disclosure relates to a composition, e.g., pharmaceutical composition or a medicament, comprising an effective amount of an anti-C5 antibody or an antigen binding fragment thereof, comprising heavy chain complementarity determining regions (HCDRs) comprising HCDR1, HCDR2 and HCDR3 sequences as set forth in SEQ ID NOs:19, 18 and 3, respectively, and light chain complementarity determining regions (LCDRs) comprising LCDR1, LCDR2 and LCDR3 sequences as set forth in SEQ ID NOs:4, and 6, respectively, for use in the treatment of C5-mediated GN, including LN and/or IgAN, in a human patient, wherein the effective amount comprises administration of the anti-C5 antibody, or the antigen binding fragment thereof:
(a) once on Day 1 at a dose of 2400 mg, followed by a dose of 3000 mg at Week 2 and once every eight weeks thereafter to a patient weighing ≥40 to <60 kg;
(b) once on Day 1 at a dose of 2700 mg, followed by a dose of 3900 mg at Week 2 and once every eight weeks thereafter to a patient weighing ≥60 to <100 kg; or
(c) once on Day 1 at a dose of 3000 mg, followed by a dose of 5400 mg at Week 2 and once every eight weeks thereafter to a patient weighing ≥100 kg.

In some embodiments, the anti C5-antibody, or antigen binding fragment thereof, is further administered:
(a) once on Day 183 at a dose of 900 mg, followed by a dose of 3000 mg at Day 197 and once every eight weeks thereafter to a patient weighing ≥40 to <60 kg;
(b) once on Day 183 at a dose of 900 mg, followed by a dose of 3900 mg at Day 197 and once every eight weeks thereafter to a patient weighing ≥60 to <100 kg; or
(c) once on Day 183 at a dose of 900 mg, followed by a dose of 5400 mg at Day 197 and once every eight weeks thereafter to a patient weighing ≥100 kg.

In some embodiments, the disclosure relates to a composition, e.g., pharmaceutical composition or a medicament, comprising an effective amount of an anti-C5 antibody or antigen binding fragment thereof, comprising HCDR₁₋₃ comprising SEQ ID NOs:19, 18 and 3 and LCDR₁₋₃ comprising SEQ ID NOs: 4, 5 and 6, for use in the treatment of C5-mediated GN, including LN and/or IgAN, in a human patient, wherein the anti-C5 antibody further comprises a variant human Fc constant region that binds to human neonatal Fc receptor (FcRn), wherein the variant human Fc constant region comprises Met429Leu and Asn435Ser substitutions at residues corresponding to methionine 428 and asparagine 434 of a native human IgG Fc constant region, each in EU numbering. Particularly, the disclosure relates to a pharmaceutical composition or a medicament comprising an effective amount of ravulizumab (ULTOMIRIS®) or an antigen-binding fragment thereof, e.g., comprising the HCDR₁₋₃ and the LCDR₁₋₃ of ravulizumab, for use in the treatment of C5-mediated GN, including LN and/or IgAN, in a human patient.

### IV. Methods

Provided herein are methods for treating C5-mediated GN, including LN and/or IgAN, in a human patient, comprising administering to the patient an anti-C5 antibody, or antigen binding fragment thereof, wherein the anti-C5 antibody or antigen binding fragment thereof is administered (or is for administration) according to a particular clinical dosage regimen (e.g., at a particular dose amount and according to a specific dosing schedule).

In one embodiment, the dose of the anti-C5 antibody, or antigen binding fragment thereof, is based on the weight of the patient. In one embodiment, for example, 900 mg, 2400 mg, or 3000 mg of the anti-C5 antibody, or antigen binding fragment thereof, is administered to a patient weighing ≥40 to <60 kg. In another embodiment, 900 mg, 2700 mg, or 3900 mg of the anti-C5 antibody, or antigen binding fragment thereof, is administered to a patient weighing ≥60 to <100 kg. In another embodiment, 900 mg, 3000 mg, or 5400 mg of the anti-C5 antibody, or antigen binding fragment thereof, is administered to a patient weighing ≥100 kg. In certain embodiments, dosage regimens are adjusted to provide the optimum desired response (e.g., an effective response).

In another embodiment, the anti-C5 antibody, or antigen binding fragment thereof, is administered for one or more administration cycles. In one embodiment, the treatment (e.g., administration cycle) is 26 weeks. In one embodiment, the anti-C5 antibody, or antigen binding fragment thereof, is administered once on Days 1, 15, 71, 127, and 183 (e.g., of the administration cycle). In another embodiment, the anti-C5 antibody, or antigen binding fragment thereof, is administered for up to two years (e.g., at a dose of 900 mg, 2400 mg, 2700 mg, or 3000 mg).

In another embodiment, a method of treating a human patient with C5-mediated GN is provided, the method comprising administering to the patient (e.g., during an administration cycle) an effective amount of an anti-C5 antibody or antigen binding fragment thereof, comprising CDR1, CDR2 and CDR3 heavy chain sequences as set forth in SEQ ID NOs:19, 18 and 3, respectively, and CDR1, CDR2 and CDR3 light chain sequences as set forth in SEQ ID NOs:4, 5 and 6, respectively, wherein the anti-C5 antibody or antigen binding fragment thereof is administered:
(a) once on Day 1 at a dose of 2400 mg, followed by a dose of 3000 mg at Week 2 and once every eight weeks thereafter to a patient weighing ≥40 to <60 kg;
(b) once on Day 1 at a dose of 2700 mg, followed by a dose of 3900 mg at Week 2 and once every eight weeks thereafter to a patient weighing ≥60 to <100 kg; or
(c) once on Day 1 at a dose of 3000 mg, followed by a dose of 5400 mg at Week 2 and once every eight weeks thereafter to a patient weighing ≥100 kg.

In another embodiment, a method of treating a human patient with C5-mediated GN is provided, the method comprising further administering to the patient the anti-C5 antibody, or antigen binding fragment thereof:
(a) once on Day 183 at a dose of 900 mg, followed by a dose of 3000 mg at Day 197 and once every eight weeks thereafter to a patient weighing ≥40 to <60 kg;
(b) once on Day 183 at a dose of 900 mg, followed by a dose of 3900 mg at Day 197 and once every eight weeks thereafter to a patient weighing ≥60 to <100 kg; or
(c) once on Day 183 at a dose of 900 mg, followed by a dose of 5400 mg at Day 197 and once every eight weeks thereafter to a patient weighing ≥100 kg.
   In one embodiment the C5-mediated GN is LN. In another embodiment, the C5-mediated GN is IgAN.

In another embodiment, a method of treating a human patient with C5-mediated GN is provided, the method comprising administering to the patient (e.g., during an administration cycle) an effective amount of an anti-C5 antibody or antigen binding fragment thereof, comprising CDR1, CDR2 and CDR3 heavy chain sequences as set forth in SEQ ID NOs:19, 18 and 3, respectively, and CDR1, CDR2 and CDR3 light chain sequences as set forth in SEQ ID NOs:4, 5 and 6, respectively, and a variant human Fc constant region that binds to human neonatal Fc receptor (FcRn), wherein the variant human Fc constant region comprises Met429Leu and Asn435Ser substitutions at residues corresponding to methionine 428 and asparagine 434 of a native human IgG Fc constant region, each in EU numbering, wherein the anti-C5 antibody or antigen binding fragment thereof is administered:
(a) once on Day 1 at a dose of 2400 mg, followed by a dose of 3000 mg at Week 2 and once every eight weeks thereafter to a patient weighing ≥40 to <60 kg;
(b) once on Day 1 at a dose of 2700 mg, followed by a dose of 3900 mg at Week 2 and once every eight weeks thereafter to a patient weighing ≥60 to <100 kg; or
(c) once on Day 1 at a dose of 3000 mg, followed by a dose of 5400 mg at Week 2 and once every eight weeks thereafter to a patient weighing ≥100 kg.

In another embodiment, a method of treating a human patient with C5-mediated GN is provided, the method comprising further administering to the patient the anti-C5 antibody, or antigen binding fragment thereof:
(a) once on Day 183 at a dose of 900 mg, followed by a dose of 3000 mg at Day 197 and once every eight weeks thereafter to a patient weighing ≥40 to <60 kg;
(b) once on Day 183 at a dose of 900 mg, followed by a dose of 3900 mg at Day 197 and once every eight weeks thereafter to a patient weighing ≥60 to <100 kg; or
(c) once on Day 183 at a dose of 900 mg, followed by a dose of 5400 mg at Day 197 and once every eight weeks thereafter to a patient weighing ≥100 kg.
   In one embodiment the C5-mediated GN is LN. In another embodiment, the C5-mediated GN is IgAN.

In another embodiment, a method of treating a human patient with C5-mediated GN is provided, the method comprising administering to the patient (e.g., during an administration cycle) an effective amount of an anti-C5 antibody or antigen binding fragment thereof, comprising CDR1, CDR2 and CDR3 heavy chain sequences as set forth in SEQ ID NOs:19, 18 and 3, respectively, and CDR1, CDR2 and CDR3 light chain sequences as set forth in SEQ ID NOs:4, 5 and 6, respectively, wherein the anti-C5 antibody is administered to a patient weighing ≥40 to <60 kg once on Day 1 at a dose of 2400 mg, followed by a dose of 3000 mg at Week 2 and once every eight weeks thereafter.

In another embodiment, a method of treating a human patient with C5-mediated GN is provided, the method comprising administering to the patient (e.g., during an administration cycle) an effective amount of an anti-C5 antibody or antigen binding fragment thereof, comprising CDR1, CDR2 and CDR3 heavy chain sequences as set forth in SEQ ID NOs:19, 18 and 3, respectively, and CDR1, CDR2 and CDR3 light chain sequences as set forth in SEQ ID NOs:4, 5 and 6, respectively, wherein the anti-C5 antibody is administered to a patient weighing ≥60<100 kg once on Day 1 at a dose of 2700 mg, followed by a dose of 3900 mg at Week 2 and once every eight weeks thereafter.

In another embodiment, a method of treating a human patient with C5-mediated GN is provided, the method comprising administering to the patient (e.g., during an administration cycle) an effective amount of an anti-C5 antibody or antigen binding fragment thereof, comprising CDR1, CDR2 and CDR3 heavy chain sequences as set forth in SEQ ID NOs:19, 18 and 3, respectively, and CDR1, CDR2 and CDR3 light chain sequences as set forth in SEQ ID NOs:4, 5 and 6, respectively, wherein the anti-C5 antibody is administered to a patient weighing ≥100 kg once on Day 1 at a dose of 3000 mg, followed by a dose of 5400 mg at Week 2 and once every eight weeks thereafter to a patient weighing ≥100 kg.

In another embodiment, a method of treating a human patient with C5-mediated GN is provided, the method comprising administering an anti-C5 antibody, or antigen binding fragment thereof, to a patient weighing ≥40 to <60 kg:
(a) once on Day 1 at a dose of 2400 mg, followed by a dose of 3000 mg at Week 2 and once every eight weeks thereafter; and
(b) once on Day 183 at a dose of 900 mg, followed by a dose of 3000 mg on Day 197 and once every eight weeks thereafter.

In another embodiment, a method of treating a human patient with C5-mediated GN is provided, the method comprising administering an anti-C5 antibody, or antigen binding fragment thereof, to a patient weighing ≥60<100 kg:
(a) once on Day 1 at a dose of 2700 mg, followed by a dose of 3900 mg at Week 2 and once every eight weeks thereafter; and
(b) once on Day 183 at a dose of 900 mg, followed by a dose of 3900 mg on Day 197 and once every eight weeks thereafter.

In another embodiment, a method of treating a human patient with C5-mediated GN is provided, the method comprising administering an anti-C5 antibody, or antigen binding fragment thereof, to a patient weighing ≥100 kg:
(a) once on Day 1 at a dose of 3000 mg, followed by a dose of 5400 mg at Week 2 and once every eight weeks thereafter; and
(b) once on Day 183 at a dose of 900 mg, followed by a dose of 5400 mg on Day 197 and once every eight weeks thereafter.

In another embodiment, a method of treating a human patient with LN is provided, the method comprising administering to the patient (e.g., during an administration cycle) an effective amount of an anti-C5 antibody or antigen binding fragment thereof, comprising CDR1, CDR2 and CDR3 heavy chain sequences as set forth in SEQ ID NOs:19, 18 and 3, respectively, and CDR1, CDR2 and CDR3 light chain sequences as set forth in SEQ ID NOs:4, 5 and 6, respectively, wherein the anti-C5 antibody or antigen binding fragment thereof is administered:
(a) once on Day 1 at a dose of 2400 mg, followed by a dose of 3000 mg at Week 2 and once every eight weeks thereafter to a patient weighing ≥40 to <60 kg;
(b) once on Day 1 at a dose of 2700 mg, followed by a dose of 3900 mg at Week 2 and once every eight weeks thereafter to a patient weighing ≥60 to <100 kg; or
(c) once on Day 1 at a dose of 3000 mg, followed by a dose of 5400 mg at Week 2 and once every eight weeks thereafter to a patient weighing ≥100 kg.

In another embodiment, a method of treating a human patient with LN is provided, the method comprising administering to the patient (e.g., during an administration cycle) an effective amount of an anti-C5 antibody or antigen binding fragment thereof, comprising CDR1, CDR2 and CDR3 heavy chain sequences as set forth in SEQ ID NOs:19, 18 and 3, respectively, and CDR1, CDR2 and CDR3 light chain sequences as set forth in SEQ ID NOs:4, 5 and 6, respectively, and a variant human Fc constant region that binds to human neonatal Fc receptor (FcRn), wherein the variant human Fc constant region comprises Met429Leu and Asn435Ser substitutions at residues corresponding to methionine 428 and asparagine 434 of a native human IgG Fc constant region, each in EU numbering, wherein the anti-C5 antibody or antigen binding fragment thereof is administered:
(a) once on Day 1 at a dose of 2400 mg, followed by a dose of 3000 mg at Week 2 and once every eight weeks thereafter to a patient weighing ≥40 to <60 kg;
(b) once on Day 1 at a dose of 2700 mg, followed by a dose of 3900 mg at Week 2 and once every eight weeks thereafter to a patient weighing ≥60 to <100 kg; or
(c) once on Day 1 at a dose of 3000 mg, followed by a dose of 5400 mg at Week 2 and once every eight weeks thereafter to a patient weighing ≥100 kg.

In another embodiment, a method of treating a human patient with LN is provided, the method comprising administering to the patient (e.g., during an administration cycle) an effective amount of an anti-C5 antibody or antigen binding fragment thereof, comprising CDR1, CDR2 and CDR3 heavy chain sequences as set forth in SEQ ID NOs:19, 18 and 3, respectively, and CDR1, CDR2 and CDR3 light chain sequences as set forth in SEQ ID NOs:4, 5 and 6, respectively, wherein the anti-C5 antibody is administered to a patient weighing ≥40 to <60 kg once on Day 1 at a dose of 2400 mg, followed by a dose of 3000 mg at Week 2 and once every eight weeks thereafter.

In another embodiment, a method of treating a human patient with LN is provided, the method comprising administering to the patient (e.g., during an administration cycle) an effective amount of an anti-C5 antibody or antigen binding fragment thereof, comprising CDR1, CDR2 and CDR3 heavy chain sequences as set forth in SEQ ID NOs:19, 18 and 3, respectively, and CDR1, CDR2 and CDR3 light chain sequences as set forth in SEQ ID NOs:4, 5 and 6, respectively, wherein the anti-C5 antibody is administered to a patient weighing ≥60<100 kg once on Day 1 at a dose of 2700 mg, followed by a dose of 3900 mg at Week 2 and once every eight weeks thereafter.

In another embodiment, a method of treating a human patient with LN is provided, the method comprising administering to the patient (e.g., during an administration cycle) an effective amount of an anti-C5 antibody or antigen binding fragment thereof, comprising CDR1, CDR2 and CDR3 heavy chain sequences as set forth in SEQ ID NOs:19, 18 and 3, respectively, and CDR1, CDR2 and CDR3 light chain sequences as set forth in SEQ ID NOs:4, 5 and 6, respectively, wherein the anti-C5 antibody is administered to a patient weighing ≥100 kg once on Day 1 at a dose of 3000 mg, followed by a dose of 5400 mg at Week 2 and once every eight weeks thereafter to a patient weighing ≥100 kg.

In another embodiment, a method of treating a human patient with immunoglobulin A nephropathy (IgAN), is provided, the method comprising administering to the patient (e.g., during an administration cycle) an effective amount of an anti-C5 antibody or antigen binding fragment thereof, comprising CDR1, CDR2 and CDR3 heavy chain sequences as set forth in SEQ ID NOs:19, 18 and 3, respectively, and CDR1, CDR2 and CDR3 light chain sequences as set forth in SEQ ID NOs:4, 5 and 6, respectively, wherein the anti-C5 antibody or antigen binding fragment thereof is administered:
(a) once on Day 1 at a dose of 2400 mg, followed by a dose of 3000 mg at Week 2 and once every eight weeks thereafter to a patient weighing ≥40 to <60 kg;
(b) once on Day 1 at a dose of 2700 mg, followed by a dose of 3900 mg at Week 2 and once every eight weeks thereafter to a patient weighing ≥60 to <100 kg; or
(c) once on Day 1 at a dose of 3000 mg, followed by a dose of 5400 mg at Week 2 and once every eight weeks thereafter to a patient weighing ≥100 kg.

In another embodiment, a method of treating a human patient with IgAN, is provided, the method comprising administering to the patient (e.g., during an administration cycle) an effective amount of an anti-C5 antibody or antigen binding fragment thereof, comprising CDR1, CDR2 and CDR3 heavy chain sequences as set forth in SEQ ID NOs:19, 18 and 3, respectively, and CDR1, CDR2 and CDR3 light chain sequences as set forth in SEQ ID NOs:4, 5 and 6, respectively, and a variant human Fc constant region that binds to human neonatal Fc receptor (FcRn), wherein the variant human Fc constant region comprises Met429Leu and Asn435Ser substitutions at residues corresponding to methionine 428 and asparagine 434 of a native human IgG Fc constant region, each in EU numbering, wherein the anti-C5 antibody or antigen binding fragment thereof is administered:
(a) once on Day 1 at a dose of 2400 mg, followed by a dose of 3000 mg at Week 2 and once every eight weeks thereafter to a patient weighing ≥40 to <60 kg;
(b) once on Day 1 at a dose of 2700 mg, followed by a dose of 3900 mg at Week 2 and once every eight weeks thereafter to a patient weighing ≥60 to <100 kg; or
(c) once on Day 1 at a dose of 3000 mg, followed by a dose of 5400 mg at Week 2 and once every eight weeks thereafter to a patient weighing ≥100 kg.

In another embodiment, a method of treating a human patient with IgAN, is provided, the method comprising further administering to the patient the anti-C5 antibody, or antigen binding fragment thereof:
(a) once on Day 183 at a dose of 900 mg, followed by a dose of 3000 mg at Day 197 and once every eight weeks thereafter to a patient weighing ≥40 to <60 kg;
(b) once on Day 183 at a dose of 900 mg, followed by a dose of 3900 mg at Day 197 and once every eight weeks thereafter to a patient weighing ≥60 to <100 kg; or
(c) once on Day 183 at a dose of 900 mg, followed by a dose of 5400 mg at Day 197 and once every eight weeks thereafter to a patient weighing ≥100 kg.

In another embodiment, a method of treating a human patient with IgAN is provided, the method comprising administering an anti-C5 antibody, or antigen binding fragment thereof, to a patient weighing ≥40 to <60 kg:
(a) once on Day 1 at a dose of 2400 mg, followed by a dose of 3000 mg at Week 2 and once every eight weeks thereafter; and
(b) once on Day 183 at a dose of 900 mg, followed by a dose of 3000 mg on Day 197 and once every eight weeks thereafter.

In another embodiment, a method of treating a human patient with IgAN is provided, the method comprising administering an anti-C5 antibody, or antigen binding fragment thereof, to a patient weighing ≥60<100 kg:
(a) once on Day 1 at a dose of 2700 mg, followed by a dose of 3900 mg at Week 2 and once every eight weeks thereafter; and
(b) once on Day 183 at a dose of 900 mg, followed by a dose of 3900 mg on Day 197 and once every eight weeks thereafter.

In another embodiment, a method of treating a human patient with IgAN is provided, the method comprising administering an anti-C5 antibody, or antigen binding fragment thereof, to a patient weighing ≥100 kg:
(a) once on Day 1 at a dose of 3000 mg, followed by a dose of 5400 mg at Week 2 and once every eight weeks thereafter; and
(b) once on Day 183 at a dose of 900 mg, followed by a dose of 5400 mg on Day 197 and once every eight weeks thereafter.

In one embodiment, the patient has not previously been treated with eculizumab. In another embodiment, the patient has previously been treated with eculizumab. In another embodiment, the patient has previously been treated with eculizumab and Day 1 (e.g., of the administration cycle) is two weeks or more from the patient's last dose of eculizumab.

In one embodiment, the patient has not previously been treated with eculizumab. In another embodiment, the patient has previously been treated with eculizumab. In another embodiment, the patient has previously been treated with eculizumab and Day 1 (e.g., of the administration cycle) is two weeks or more from the patient's last dose of eculizumab.

In another embodiment, the patient is an IgAN patient who has previously been treated with a renin-angiotensin system (RAS) inhibiting medication, such as an angiotensin-converting enzyme (ACE) inhibitor or angiotensin II receptor blocker (ARB).

In some embodiments, the disclosure relates to use of an effective amount of an anti-C5 antibody or antigen binding fragment thereof, comprising heavy chain complementarity determining regions (HCDRs) comprising HCDR1, HCDR2 and HCDR3 sequences as set forth in SEQ ID NOs:19, 18 and 3, respectively, and light chain complementarity determining regions (LCDRs) comprising LCDR1, LCDR2 and LCDR3 sequences as set forth in SEQ ID NOs:4, 5 and 6, respectively, in the manufacture of a composition, e.g., pharmaceutical composition or a medicament, for treating C5-mediated GN, including LN and/or IgAN, in a human patient, wherein the effective amount comprises administration of the anti-C5 antibody or the antigen binding fragment thereof:
(a) once on Day 1 at a dose of 2400 mg, followed by a dose of 3000 mg at Week 2 and once every eight weeks thereafter to a patient weighing ≥40 to <60 kg;
(b) once on Day 1 at a dose of 2700 mg, followed by a dose of 3900 mg at Week 2 and once every eight weeks thereafter to a patient weighing ≥60 to <100 kg; or
(c) once on Day 1 at a dose of 3000 mg, followed by a dose of 5400 mg at Week 2 and once every eight weeks thereafter to a patient weighing ≥100 kg.

In some embodiments, the anti C5-antibody, or antigen binding fragment thereof, is further administered:
(a) once on Day 183 at a dose of 900 mg, followed by a dose of 3000 mg at Day 197 and once every eight weeks thereafter to a patient weighing ≥40 to <60 kg;
(b) once on Day 183 at a dose of 900 mg, followed by a dose of 3900 mg at Day 197 and once every eight weeks thereafter to a patient weighing ≥60 to <100 kg; or
(c) once on Day 183 at a dose of 900 mg, followed by a dose of 5400 mg at Day 197 and once every eight weeks thereafter to a patient weighing ≥100 kg.

In some embodiments, the disclosure relates to use of an effective amount of an anti-C5 antibody or antigen binding fragment thereof comprising HCDR₁₋₃ comprising SEQ ID NOs:19, 18 and 3 and LCDR₁₋₃ comprising SEQ ID NOs: 4, 5 and 6, in the manufacture of a composition, e.g., pharmaceutical composition or a medicament, for treating C5-mediated GN, including LN and/or IgAN, in a human patient, wherein the anti-C5 antibody further comprises a variant human Fc constant region that binds to human neonatal Fc receptor (FcRn), wherein the variant human Fc constant region comprises Met429Leu and Asn435Ser substitutions at residues corresponding to methionine 428 and asparagine 434 of a native human IgG Fc constant region, each in EU numbering. Particularly, the disclosure relates to use of an effective amount of ravulizumab (ULTOMIRIS®) or an antigen-binding fragment thereof, e.g., comprising the HCDR₁₋₃ and the LCDR₁₋₃ of ravulizumab, in the manufacture of a composition, e.g., pharmaceutical composition or a medicament, for C5-mediated GN, including LN and/or IgAN, in a human patient.

### V. Outcomes

Provided herein are methods for treating C5-mediated GN, including LN and/or IgAN, in a patient comprising administering to the patient an anti-C5 antibody.

Symptoms of LN include, but are not limited to, e.g., foamy urine (due to proteinuria, excess protein in urine), edema (e.g., in the hands, ankles or feet), high blood pressure (hypertension), kidney inflammation, kidney impairment, joint pain or swelling, muscle pain, fever with no known cause, high levels of creatinine in the blood, and/or a red rash (e.g., often on the face, across the nose and cheeks, sometimes called a butterfly rash because of its shape).

Symptoms of IgAN include, but are not limited to, e.g., hematuria (blood in the urine that can sometimes make it pink, dark brown or cola colored), edema (e.g., in the hands, ankles or feet), pain on the side of the back (flank pain), high blood pressure (hypertension), and/or foamy urine (due to proteinuria, excess protein in urine).

In one embodiment, patients treated according to the disclosed methods maintain a serum trough concentration of the anti-C5 antibody, or antigen binding fragment thereof, of at least 150, 155, 160, 165, 170, 175, 180, 185, 190, 200, 205, 210, 215, 220, 225, 230, 240, 245, 250, 255, 260, 265, 270, 280, 290, 300, 305, 310, 315, 320, 325, 330, 335, 340, 345, 350, 355, 360, 365, 370, 375, 380, 385, 390, 395 or 400 μg/mL or greater. In one embodiment, patients treated according to the disclosed methods maintain a serum trough concentration of the anti-C5 antibody, or antigen binding fragment thereof, of at least 175 μg/mL or greater.

In one embodiment, patients treated according to the disclosed methods have a free C5 concentration of 0.5 μg/mL or less (e.g., 0.4 μg/mL, 0.3 μg/mL, 0.2 μg/mL, or 0.1 μg/mL or less).

The efficacy of the treatment methods provided herein can be assessed using any suitable means. In one embodiment, the treatment results in a shift towards normal levels of one or more renal injury biomarkers selected from the group consisting of CD163, MCP-1, and EGF.

In another embodiment, the treatment results in a shift towards normal levels of one or more biomarkers selected from the group consisting of sC5b-9, Factor Ba, Factor Bb, C5a, C3c, C3, C4d, CD68, properdin, complement component 9 [C9], C1q, C5aR, and creatinine.

In another embodiment, the treatment results in a change in Estimated glomerular filtration rate (eGFR) compared to baseline.

In another embodiment, the treatment results in a change in serum albumin compared to baseline.

In another embodiment, the treatment results in a reduction in proteinuria compared to baseline. In another embodiment, the patient has an estimated glomerular filtration rate (eGFR)≥30 mL/min/1.73m2 and proteinuria prior to treatment. In another embodiment, the proteinuria for an LN patient is a urine protein to creatinine ratio (UPCR)≥1 g/g from one 24-hr urine collection. In another embodiment, proteinuria for an IgAN patient is a mean protein≥1 g/24-hr from 2 valid 24-hr collections. In another embodiment, the treatment results in a 25%, 30%, 35%, 40%, 45%, 50%, 55%, 60%, 65%, 70%, 75%, 80%, 85%, 90%, or 95% reduction in proteinuria compared to baseline. In another embodiment, the reduction in proteinuria occurs at 6 weeks, 8 weeks, 10 weeks, 12 weeks, 14 weeks, 16 weeks, 18 weeks, 20 weeks, 22 weeks, 24 weeks, 26 weeks, 28 weeks, or 30 weeks after treatment compared to baseline. In another embodiment, proteinuria is measured by a complete 24-hour urine collection.

In another embodiment, the treatment results in a reduction or cessation in one or more of the following symptoms compared to baseline in an LN patient: foamy urine, proteinuria, edema, high blood pressure, kidney inflammation, kidney impairment, joint pain, joint swelling, muscle pain, fever with no known cause, high levels of creatinine in the blood, and/or a red rash.

In another embodiment, the treatment results the LN patient has an active flare prior to treatment.

In another embodiment, the treatment results in a Complete Renal Response (CRR) in an LN patient. In another embodiment, the CRR comprises:
(a) a decrease in mean urine protein-to-creatinine ratio (UPCR) to ≤0.5 g/g based on two 24-hour urine collections;
(b) an Estimated glomerular filtration rate (eGFR)≥60 mL/min/1.73 m2 or no eGFR reduction>20% from the baseline value based on mean of 2 values; and
(c) no treatment failure.

In another embodiment, the treatment results in a Partial Renal Response (PRR) in an IgAN patient. In another embodiment, the PRR comprises:
(a) a decrease in UPCR>50% compared to the baseline value based on mean of two 24 hour urine collections;
(b) an Estimated glomerular filtration rate (eGFR)>60 mL/min/1.73 m2 or no eGFR reduction≥20% from the baseline value based on mean of 2 values; and
(c) no treatment failure.

In another embodiment, the treatment prevents a renal flare in an LN patient, wherein:
(a) renal flare for a patient who has achieved CRR is reproducible recurrence of proteinuria≥1 g/g; and
(b) renal flare for a patient who has not achieved CRR is:
   (i) a reproducible increase of serum creatinine>25% higher than baseline or above the upper limit of normal, including any one of the following:
      a. reproducible proteinuria≥75% higher than baseline;
      b. worsening active urinary sediment compared to baseline as defined by an increase of ≥5 RBCs/high power field (hpf) or new RBC casts (based on local laboratory results from at least 2 samples); and/or
      c. kidney biopsy newly conducted since the biopsy used for eligibility demonstrating LN Class III or IV activity;
   (ii) a reproducible doubling of the UPCR from a 24 hour urine collection compared with the lowest previous value obtained after the first dose of the anti-C5 antibody, or antigen binding fragment thereof.

In another embodiment, the treatment prevents an Extrarenal SLE Flare in a LN patient, wherein the Extrarenal SLE Flare comprises an increase in Systemic Lupus Erythematosus Disease Activity Index Safety of Estrogen in Lupus Erythematosus National Assessment (SELENA) Modification (SLEDAI-2K)≥4 points that is not accounted for by proteinuria, hematuria, urinary cellular casts, hypocomplementemia, or an increase in anti-double-stranded DNA (anti-dsDNA) antibody level.

In another embodiment, the treatment results in Partial Remission (PR) in an IgAN patient. In another embodiment, the PR comprises mean proteinuria<1 g/24-hours based on 2 valid 24-hour urine collections.

In another embodiment, the treatment results in a reduction or cessation in one or more of the following symptoms in an IgAN patient compared to baseline: hematuria, dark brown or cola colored urine, edema, flank pain, hypertension, foamy urine, and/or proteinuria.

In another embodiment, the treatment results in an improvement in the patient's quality of life, as assessed by European Quality of Life Health 5-item questionnaire dimensions 5 level (EQ-5D-5L) and/or Short Form (36) Health Survey (SF-36) total score (e.g., for an LN and/or IgAN patient).

In another embodiment, the treatment results in an improvement in the patient's quality of life, as assessed by Functional Assessment of Chronic Therapy (FACIT)-Fatigue score (e.g., for an LN patient).

In another embodiment, the treatment results in terminal complement inhibition.

In another embodiment, the treatment results in a reduction in adverse events.

### VI. Kits and Unit Dosage Forms

Also provided herein are kits that include a pharmaceutical composition containing an anti-C5 antibody or antigen binding fragment thereof, such as ravulizumab or BNJ421, and a pharmaceutically acceptable carrier, in a therapeutically effective amount adapted for use in the preceding methods. The kits optionally also can include instructions, e.g., comprising administration schedules, to allow a practitioner (e.g., a physician, nurse, or patient) to administer the composition contained therein to administer the composition to a patient having C5-mediated GN (e.g., LN and/or IgAN). The kit also can include a syringe.

Optionally, the kits include multiple packages of the single-dose pharmaceutical compositions each containing an effective amount of the anti-C5 antibody, or antigen binding fragment thereof, for a single administration in accordance with the methods provided above. Instruments or devices necessary for administering the pharmaceutical composition(s) also may be included in the kits. For instance, a kit may provide one or more pre-filled syringes containing an amount of the anti-C5 antibody or antigen binding fragment thereof.

In one embodiment, a kit for treating C5-mediated GN (e.g., LN and/or IgAN) in a human patient comprises: (a) a dose of an anti-C5 antibody or antigen binding fragment thereof, comprising CDR1, CDR2 and CDR3 domains of the heavy chain variable region having the sequence set forth in SEQ ID NO:12, and CDR1, CDR2 and CDR3 domains of the light chain variable region having the sequence set forth in SEQ ID NO:8; and (b) instructions for using the anti-C5 antibody, or antigen binding fragment thereof, according to any of the methods described herein.

In one embodiment, the kit comprises a dose of an anti-C5 antibody or antigen binding fragment thereof, wherein the anti-C5 antibody, or antigen binding fragment thereof, is administered:
(a) once on Day 1 at a dose of 2400 mg, followed by a dose of 3000 mg at Week 2 and once every eight weeks thereafter to a patient weighing ≥40 to <60 kg;
(b) once on Day 1 at a dose of 2700 mg, followed by a dose of 3900 mg at Week 2 and once every eight weeks thereafter to a patient weighing ≥60 to <100 kg; or
(c) once on Day 1 at a dose of 3000 mg, followed by a dose of 5400 mg at Week 2 and once every eight weeks thereafter to a patient weighing ≥100 kg.

In another embodiment, the kit comprises a dose of an anti-C5 antibody or antigen binding fragment thereof, wherein the anti-C5 antibody, or antigen binding fragment thereof, is administered:
(d) once on Day 1 at a dose of 2400 mg, followed by a dose of 3000 mg at Week 2 and once every eight weeks thereafter and once on Day 183 at a dose of 900 mg, followed by a dose of 3000 mg on Day 197 and once every eight weeks thereafter, to a patient weighing ≥40 to <60 kg;
(e) once on Day 1 at a dose of 2700 mg, followed by a dose of 3900 mg at Week 2 and once every eight weeks thereafter and once on Day 183 at a dose of 900 mg, followed by a dose of 3900 mg on Day 197 and once every eight weeks thereafter, to a patient weighing ≥60 to <100 kg; or
(f) once on Day 1 at a dose of 3000 mg, followed by a dose of 5400 mg at Week 2 and once every eight weeks thereafter and once on Day 183 at a dose of 900 mg, followed by a dose of 5400 mg on Day 197 and once every eight weeks thereafter, to a patient weighing ≥100 kg.

The following examples are merely illustrative and should not be construed as limiting the scope of this disclosure in any way as many variations and equivalents will become apparent to those skilled in the art upon reading the present disclosure. The contents of all references, Genbank entries, patents and published patent applications cited throughout this application are expressly incorporated herein by reference.

### EXAMPLE

### Example 1: A Phase 2, Double-Blind, Randomized, Placebo-Controlled Study to Evaluate the Efficacy and Safety of Ravulizumab in Adult Participants with Proliferative Lupus Nephritis (LN) or Immunoglobulin A Nephropathy (IgAN)

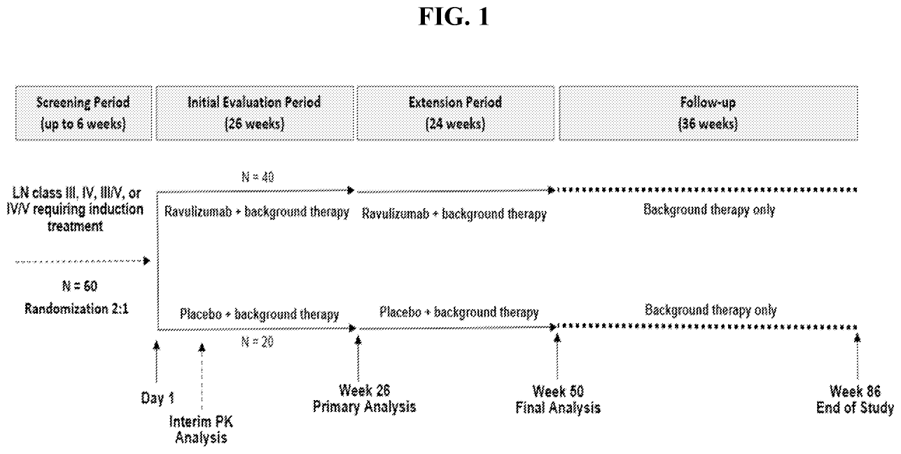

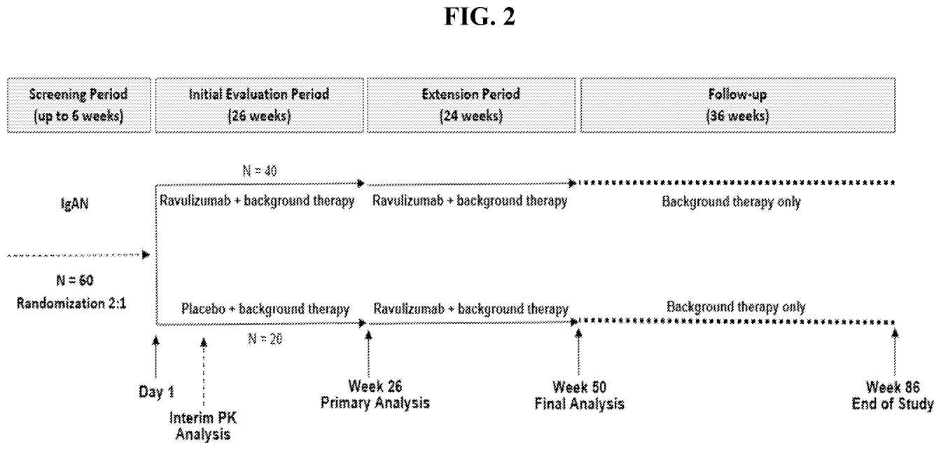

A Phase 2, randomized, double-blind, placebo-controlled, multicenter study (referred to as “Study ALXN1210 NEPH 202”) of ravulizumab in addition to background therapy consistent with the standard of care is conducted in 120 adult participants (18 to 75 years of age) with either LN or IgAN, as discussed below and as further detailed in Example 2. The study design for the LN cohort is set forth in FIG. 1. The study design for the IgAN cohort is set forth in FIG. 2.

1. Objectives

The primary objective of the study for both cohorts is to evaluate the efficacy of ravulizumab compared with placebo to reduce proteinuria in adult participants with LN or IgAN. This objective is assessed based on percentage change in proteinuria from baseline to Week 26 (based on 24-hr urine collection[s] at each time point).

A secondary objective for both cohorts is to evaluate the efficacy of ravulizumab compared with placebo to improve measures of kidney function in adult participants with LN or IgAN (e.g., via (1) percentage change in proteinuria from baseline to Week 50 (based on 24-hr urine collection[s] at each time point), (2) percentage of participants with >30% and >50% reduction in proteinuria at Week 26 and Week 50 compared to baseline (based on 24-hr urine collection[s] at each time point), (3) change from baseline in eGFR at Week 26 and Week 50, and (4) absolute values and change from baseline in serum C3 and C4 concentrations at Week 26 and Week 50).

A secondary objective for the LN cohort only is to evaluate the efficacy of ravulizumab compared with placebo to improve measures of kidney function in adult participants with LN (e.g., via (1) percentage of participants meeting the criteria for CRR at Week 26 and Week 50, (2) percentage of participants meeting the criteria for PRR at Week 26 and Week 50, (3) time to UPCR<0.5 g/g as measured by spot urine sample, (4) percentage of participants achieving corticosteroid taper to 7.5 mg/day at Weeks 14, 26, and 50, (5) Percentage of participants with Renal Flare through Week 50, (6) Percentage of participants with Extrarenal SLE Flare through Week 50, (7) percentage of participants with Treatment Failure through Week 50, and (8) absolute values and change from baseline in serum albumin at Week 26 and Week 50.

A secondary objective for the IgAN cohort only is to evaluate the efficacy of ravulizumab compared with placebo on measures of kidney function in adult participants with IgAN (e.g., via percentage of participants meeting the criteria for Partial Remission at Week 26 and Week 50).

PK/PD/Immunogenicity objectives for both cohorts include characterizing the PK/PD of ravulizumab in adult participants with LN or IgAN (e.g., via (1) absolute values and change from baseline in total C5 and free C5 concentrations over time and (2) absolute values and change from baseline in ravulizumab concentrations over time), as well as characterizing the potential for immunogenicity of ravulizumab in adult participants with LN or IgAN (e.g., incidence of ADAs over time).

A safety objective for both cohorts is to characterize the safety and tolerability of ravulizumab in adult participants with LN or IgAN (e.g., via incidence of AEs and SAEs over time).

Exploratory objectives for both cohorts include (1) evaluating the efficacy of ravulizumab compared with placebo on hematuria in adult participants with LN or IgAN (e.g., effect on hematuria as measured by absolute value and change from baseline in RBC in urine from baseline to Week 26 and Week 50 and percentage of participants with >10 RBC), (2) assessing quality of life based on participant reported outcomes in adult participants with LN or IgAN based on treatment with ravulizumab compared with placebo (e.g., change from baseline in SF-36 at Week 26 and Week 50 and change from baseline in EQ-5D-5L at Week 26 and Week 50), (3) evaluating complement and autoimmune biomarkers in adult participants with LN or IgAN (e.g., absolute values and change from baseline in levels of biomarkers in blood, urine, and kidney tissue at Week 26 and Week 50).

Exploratory objectives for the LN cohort only include (1) assessing the efficacy of ravulizumab in exploratory efficacy endpoints (e.g., time to CRR and PRR (using spot UPCR), percentage of participants with Overall Renal Response at Week 26 and Week 50 (CRR and PRR), and time to UPCR>50% decrease from baseline (using spot UPCR)), (2) assessing quality of life based on participant reported outcomes (change from baseline in FACIT Fatigue score at Week 26 and Week 50), and (3) assessing the efficacy of ravulizumab in other exploratory endpoints (absolute values and change from baseline in anti-dsDNA and anti-C1q antibodies at Week 26 and Week 50 and histology changes from baseline to Week 50).

Exploratory objectives for the IgAN cohort only include assessing the efficacy of ravulizumab in exploratory efficacy endpoints (slope of eGFR computed from baseline to Week 26 and Week 50).

2. Overall Design

This is a Phase 2, randomized, double-blind, placebo-controlled, multicenter study of ravulizumab in addition to background therapy consistent with the standard of care in adult participants (18 to 75 years of age) with either lupus nephritis (LN) or IgAN. All participants are naive to complement inhibitor treatment and have either a diagnosis of LN with an active flare or IgAN based on kidney biopsy, eGFR≥30 mL/min/1.73 m2, and proteinuria [defined as urine protein to creatinine ratio (UPCR)≥1 g/g from one 24-hr urine collection (LN cohort) or as mean protein≥1 g/24-hr from 2 valid 24-hr collections (IgAN cohort)]. Participants in the IgAN cohort have been treated with stable doses of the maximum tolerated RAS inhibiting medications and have controlled, stable blood pressure (<140/90 mmHg) for ≥3 months prior to Screening.

The study consists of an up to 6-week Screening Period, a 26-week Initial Evaluation Period, a 24-week Extension Period, and a 36 week post-treatment Follow-up Period. Thus, the total treatment duration is 50 weeks and the total study duration is up to 86 weeks.

Participants are screened for eligibility for up to 6 weeks during the Screening Period. Approximately 120 adult participants with either LN or IgAN are enrolled into the study. For each disease cohort, 60 participants are randomly assigned in a 2:1 ratio to receive ravulizumab or placebo (40 ravulizumab, 20 placebo). Randomization is stratified by whether corticosteroid induction treatment was initiated prior to Screening versus during the Screening Period for participants in the LN cohort and by mean proteinuria (1 to 2 g/day versus >2 g/day) from 2 valid 24-hr urine collections during the Screening Period for participants in the IgAN cohort.

For participants in the LN cohort, all screening laboratory assessments are performed as soon as possible after signing of the informed consent form (ICF). All participants in the LN cohort are randomized as soon as possible once eligibility is confirmed.

All participants are required to have meningococcal vaccination, however for participants in the IgAN cohort, every effort should be made to start the meningococcal vaccination series at least 14 days prior to randomization in order to avoid antibiotic prophylaxis and minimize the potential triggering of innate immunity with possible effects on proteinuria and hematuria.

During the Initial Evaluation Period, all participants receive a weight based loading dose of ravulizumab or placebo on Day 1, followed by weight-based maintenance doses of ravulizumab or placebo on Day 15 and then q8w thereafter (see Table 2). All participants receive background therapy consistent with the standard of care for participants with LN and IgAN throughout the study.

During the 24-week Extension Period, participants continue to receive study drug (ravulizumab or placebo):
1. Participants in the LN cohort continue to receive their randomized allocation of study drug (ravulizumab or placebo) q8w until the end of the Extension Period.
2. Participants in the IgAN cohort randomized to the placebo group switch to receive a blinded loading dose of ravulizumab at Week 26 and then open label weight based dosing of ravulizumab q8w until the end of the Extension Period.
3. Participants in the IgAN cohort randomized to the ravulizumab group receive a blinded dose of 900 mg ravulizumab and then open-label weight based dosing of ravulizumab q8w until the end of the Extension Period.

During the 36-week Post treatment Follow-up Period, participants continue to receive standard of care, at the discretion of the Investigator, and are monitored for clinical events of interest and kidney function.

All participants, including participants who discontinue study drug early, are followed for safety until 8 weeks after the last dose of study drug. The end of study is defined as the last participant's last visit in the Post-treatment Follow-up Period.

To ensure the adequacy of the dose regimen, an interim pharmacokinetics (PK)/pharmacodynamics (PD) analysis for dose confirmation is conducted by an independent clinical pharmacologist. The interim PK is conducted using masked PK/PD data from the first participants treated with ravulizumab (a minimum of 3 participants in each disease-specific cohort). In the event of dose adjustments, the participants treated with the previous dose switch over to the new dose and continue treatment on study but are excluded from the primary efficacy analysis. Replacement participants can be enrolled to preserve study power.

3. Inclusion and Exclusion Criteria

To be eligible to participate in the study, participants in both cohorts must meet all of the below criteria.
1. Participant must be ≥18 and ≤75 years of age at the time of signing the informed consent;
2. Body weight≥40 kg at Screening;
3. Male or female. Female participants of childbearing potential, male participants, and male participants with female partners of childbearing potential must follow protocol specified contraception guidance;
4. Capable of giving informed consent;
5. To reduce the risk of meningococcal infection (N meningitidis), all participants must be vaccinated against meningococcal infection from serogroups A, C, W, Y, and B within 3 years prior to, or at the time of, randomization according to national/local guidelines. Participants who do not meet this requirement are vaccinated against meningococcal infection prior to randomization according to national/local guidelines and receive prophylactic antibiotics for at least 2 weeks after meningococcal vaccination if randomization occurs <2 weeks after initial vaccination;
6. All participants must also receive vaccinations for Haemophilus influenzae type b (Hib) and Streptococcus pneumoniae prior to randomization, unless previously vaccinated, according to current national/local vaccination guidelines;
7. Local pathology report from the biopsy used for diagnosis must be available; and
8. Participants on SGLT-2 inhibitors (e.g., empagliflozin) must be on a stable dose for ≥3 months with no planned change in dose during the study.

In addition, to be included in the LN Cohort, the participant must meet the following criteria:
1. Clinical diagnosis of SLE by 2019 The American College of Rheumatology (ACR) and European League Against Rheumatism (EULAR) criteria;
2. Diagnosis of 2018 Revised ISN/RPS classification (active focal or diffuse proliferative LN Class III or IV confirmed by biopsy obtained ≤6 months prior to Screening or during Screening Period. Participants may co-exhibit Class V disease. Participants with de novo or relapsing disease may be eligible;
3. Clinically active LN at Screening requiring/receiving immunosuppression induction treatment in the opinion of the Investigator; and
4. Proteinuria with UPCR≥1 g/g based on one 24-hour urine collection during the Screening Period.

In addition, to be included in the IgAN Cohort, the participant must meet the following criteria:
1. Established diagnosis of primary IgAN based on kidney biopsy obtained any time prior to or during the Screening Period;
2. Mean proteinuria≥1 g/day on 2 complete and valid 24-hour urine collections during the Screening Period;
3. Presence of hematuria as defined by 1+blood based on urine dipstick or ≥10 red blood cell (RBC)/hpf microscopy on urine sediment (performed by the local laboratory);
4. Compliance with stable and optimal dose of RAS inhibitor treatment including maximum allowed or tolerated ACE inhibitor and/or angiotensin receptor blocker dose for ≥3 months prior to Screening with no expected change in dose during the study; and
5. Controlled and stable blood pressure over the past 3 months <140/90 mmHg.

Participants from both cohorts are excluded from the study if any of the below criteria apply.
1. Estimated GFR<30 mL/min/1.73 m2 during Screening calculated by CKD-EPI;
2. More than or equal to 50% interstitial fibrosis, tubular atrophy, glomerular sclerosis, or crescent formation in glomeruli on most recent kidney biopsy prior or during the Screening Period;
3. Concomitant significant renal disease other than LN or IgAN on the most recent biopsy prior to or during the Screening Period;
4. History of kidney transplant or planned kidney transplant during the Treatment Period;
5. History of other solid organ (heart, lung, small bowel, pancreas, or liver) or bone marrow transplant; or planned transplant during the Treatment Period;
6. Splenectomy or functional asplenia;
7. Known medical or psychological condition(s) or risk factor that, in the opinion of the Investigator, might interfere with the participant's full participation in the study, pose any additional risk for the participant, or confound the assessment of the participant or outcome of the study;
8. Known or suspected history of drug or alcohol abuse or dependence within 1 year prior to the start of the Screening Period;
9. History of malignancy within 5 years of Screening with the exception of nonmelanoma skin cancer or carcinoma in situ of the cervix that has been treated with no evidence of recurrence;
10. Known history of hepatitis B or C viral infection;
11. Known history of HIV infection (evidenced by HIV type 1 or type 2 [HIV 1, HIV 2] antibody);
12. Bone marrow insufficiency with absolute neutrophil count<1.3×103/μL; thrombocytopenia (platelet count<50,000/mm3);
13. Active systemic bacterial, viral, or fungal infection within 14 days prior to randomization;
14. History of N meningitidis infection;
15. Inability to take or tolerate the standard of care background therapies;
16. Received biologic, including but not limited to belimumab or rituximab, ≤6 months prior to Screening;
17. Previously received a complement inhibitor (e.g., eculizumab) at any time;
18. Participation in another investigational drug or investigational device study within 30 days before initiation of study drug on Day 1 in this study or within 5 half-lives of that investigational product, whichever is greater; or
19. Pregnant, breastfeeding, or intending to conceive during the course of the study.

In addition, participants from the LN cohort are excluded from the study if any of the below criteria apply.
1. Participants who have received any of the following treatments after their qualifying kidney biopsy used for eligibility: cyclophosphamide ≤6 months of Screening, calcineurin inhibitors ≤3 months of Screening, a cumulative dose of IV methylprednisolone>3 g, mycophenolate mofetil>2 g/day (or equivalent) for ≥4 consecutive weeks, or oral corticosteroids≥0.5 mg/kg/day for ≥4 consecutive weeks;
2. Uncontrolled hypertension (systolic blood pressure>160 or diastolic blood pressure>110 mmHg) on 2 or more measurements during the Screening Period; or
3. Clinically active SLE-related cerebritis, seizures, pericarditis, stroke, or stroke syndrome requiring treatment.

In addition, participants from the IgAN cohort are excluded from the study if any of the below criteria apply.
1. Diagnosis of rapid progressive glomerulonephritis as measured by eGFR loss ≥30% over a period of 3 months prior to or during the Screening Period;
2. Secondary etiologies of IgAN (eg, SLE, cirrhosis, celiac disease);
3. Clinically active Henoch-Schonlein purpura (IgA vasculitis) requiring treatment
4. Prednisone or prednisone equivalent >20 mg for >14 consecutive days or any other immunosuppression within 6 months of Screening;
5. Blood pressure of ≥140/90 mmHg during the Screening Period confirmed on 2 measures >30 minutes apart; or
6. Body mass index≥35.

4. Study Drug

Ravulizumab is formulated at pH 7.0 and is supplied in 30 mL single-use vials. Each vial of ravulizumab contains 300 mg of ravulizumab (10 mg/mL) in 10 mM sodium phosphate, 150 mM sodium chloride, 0.02% polysorbate 80, and water for injection. The comparator product (placebo) is formulated as a matching sterile, clear, colorless solution with the same buffer components, but without active ingredient. Additional details are presented in Table 1.

TABLE 1
Study Drug

| Study Drug | | |
|---|---|---|
| Name | Ravulizumab | Placebo |
| Dose | Vial | Vial |
| formulation | | |
| Physical | Liquid solution practically | Liquid solution practically |
| description | free from particles | free from particles |
| Unit dose | 300 mg (10 mg/mL | Placebo |
| Strength(s) | concentrated solution) | |
| Route of | IV infusion | IV infusion |
| administration | | |
| Use | Experimental | Placebo comparator |

Abbreviations:
IV = intravenous

The dosing regimen (Table 2) consists of a loading dose followed by maintenance dosing administered q8w. The maintenance dosing is initiated 2 weeks after the loading dose administration. Weight-based dosing is based on the participant's body weight recorded at the day of the infusion visit. If the weight at the day of the infusion cannot be obtained, the weight recorded during the most recent prior study visit can be used.

TABLE 2
Weight-based Doses of Ravulizumab

| Body Weight Range | Loading | Maintenance |
|---|---|---|
| (kg)^{a} | Dose (mg) | Dose (mg) |
| ≥40 to <60 | 2400 | 3000 |
| ≥60 to <100 | 2700 | 3900 |
| ≥100 | 3000 | 5400 |

^{a}Dose regimen will be based on the last recorded study visit body weight. If the study drug is prepared the night before a visit, the weight from the most recent study visit should be used.

At the scheduled dosing visits, study drug is administered after all other tests and procedures have been completed, excluding the postdose sample collections (PK/PD/biomarkers).

During the Initial Evaluation Period (Day 1 through Week 26), participants in each cohort are randomized 2:1 to receive blinded doses of ravulizumab or placebo.
Ravulizumab group: participants receive a blinded loading dose of ravulizumab via IV infusion on Day 1, followed by a blinded maintenance doses at Week 2 then q8w thereafter through the end of the Initial Evaluation Period
Participants in the placebo group receive a blinded matching placebo dose via IV infusion on Day 1, followed by a blinded matching placebo dose at Week 2, then q8w thereafter through the end of the Initial Evaluation Period.

During the Extension Period (Week 26 through Week 50), participants in the LN cohort continue on the same maintenance regimen. In the IgAN cohort, participants in the placebo group switch to receive a blinded loading dose of ravulizumab at Week 26 and participants in the ravulizumab group receive a blinded ravulizumab dose of 900 mg at Week 26. Starting at Week 28, all participants in the IgAN cohort receive open-label weight-based doses of ravulizumab (Table 3) q8w until the end of the Extension Period.

TABLE 3
Reference Chart for Weight-Based Dosing in IgAN Cohort

| | | | | | | | |
|---|---|---|---|---|---|---|---|
| | | | | | | Diluent | |
| | | | | | | (0.9% | |
| | | | | | | Sodium | |
| | Ravulizumab | Body | Ravulizumab | Ravulizumab | Placebo | Chloride) | Total |
| Study | or Placebo | Weight | Dose | Volume | Volume | Volume | Volume |
| Period | Dosing | (kg)^{1a} | (mg) | (mL) | (mL) | (mL) | (mL) |

Ravulizumab Group

| | | | | | | | |
|---|---|---|---|---|---|---|---|
| Initial | Loading dose | ≥40 | 2400 | 240 | 0 | 240 | 480 |
| Evaluation | (Day 1) | to <60 | | | | | |
| Period | | ≥60 to | 2700 | 270 | 0 | 270 | 540 |
| | | <100 | | | | | |
| | | ≥100 | 3000 | 300 | 0 | 300 | 600 |
| | Maintenance | ≥40 to | 3000 | 300 | 0 | 300 | 600 |
| | dose | <60 | | | | | |
| | (Days 15, 71, | ≥60 to | 3900 | 390 | 0 | 390 | 780 |
| | 127) | <100 | | | | | |
| | | ≥100 | 5400 | 540 | 0 | 540 | 1080 |
| Extension | Blinded dose^{b} | ≥40 to | 900 | 90 | 150 | 240 | 480 |
| Period | (Day 183) | <60 | | | | | |
| | | ≥60 to | 900 | 90 | 180 | 270 | 540 |
| | | <100 | | | | | |
| | | ≥100 | 900 | 90 | 210 | 300 | 600 |
| | Maintenance | ≥40 to | 3000 | 300 | 0 | 300 | 600 |
| | dose | <60 | | | | | |
| | (Days 197 to | ≥60 to | 3900 | 390 | 0 | 390 | 780 |
| | 351 q8w) | <100 | | | | | |
| | | ≥100 | 5400 | 540 | 0 | 540 | 1080 |

Placebo Group

| | | | | | | |
|---|---|---|---|---|---|---|
| Loading dose | ≥40 to | 0 | 0 | 240 | 240 | 480 |
| (Day 1) | <60 | | | | | |

Ravulizumab Group

| | | | | | | | |
|---|---|---|---|---|---|---|---|
| Initial | | ≥60 to | 0 | 0 | 270 | 270 | 540 |
| Evaluation | | <100 | | | | | |
| Period | | ≥100 | 0 | 0 | 300 | 300 | 600 |
| | Maintenance | ≥40 to | 0 | 0 | 300 | 300 | 600 |
| | dose | <60 | | | | | |
| | (Days 15, 71, | ≥60 to | 0 | 0 | 390 | 390 | 780 |
| | 127) | <100 | | | | | |
| | | ≥100 | 0 | 0 | 540 | 540 | 1080 |
| Extension | Blinded | ≥40 to | 2400 | 240 | 0 | 240 | 480 |
| Period | loading dose^{c} | <60 | | | | | |
| | (Day 183) | ≥60 to | 2700 | 270 | 0 | 270 | 540 |
| | | <100 | | | | | |
| | | ≥100 | 3000 | 300 | 0 | 300 | 600 |
| | Maintenance | ≥40 to | 3000 | 300 | 0 | 300 | 600 |
| | dose | <60 | | | | | |
| | (Days 197 to | ≥60 to | 3900 | 390 | 0 | 390 | 780 |
| | 351, q8w) | <100 | | | | | |
| | | ≥100 | 5400 | 540 | 0 | 540 | 1080 |

^{a}Dose regimen is based on the participant's most recently recorded body weight. Contact the medical monitor if a participant's weight drops below 40 kg during the study treatment period (Initial Evaluation Period or the Extension Period).
^{b}Blinded dose on Day 183 (Week 26) for participants who were randomized to the ravulizumab group and are entering into the Extension Period.
^{c}Blinded loading dose on Day 183 (Week 26) for participants who were randomized to the placebo group and are entering into the Extension Period.

5. Background Therapy for LN Cohort

During the course of the study, participants in the LN cohort receive background therapy consistent with the standard of care for induction and maintenance treatment of LN.

For participants who have not started corticosteroid induction treatment prior to Screening:
1. Participants receive a cumulative dose of 1 gram of methylprednisolone IV administered in 1 or multiple divided doses during the Screening Period (prior to Day 1).
2. During the Screening Period and no later than Day 2, all participants receive oral corticosteroids with prednisone or prednisone equivalent 0.5 mg/kg/day. The starting minimum and maximum dose allowed are 30 mg/day and 60 mg/day, respectively. A corticosteroid taper commences on Week 2 (Day 14) as outlined in Table 4.
3. During the Screening Period and no later than Day 1, participants receive a cumulative dose of 1 to 1.5 g/day of MMF any time after completion of the IV methylprednisolone during the Screening Period and no later than Day 1. The dose can be administered in multiple divided doses. Participants continue to receive 1 to 1.5 g/day for 1 week.
4. After receiving 1 to 1.5 g/day for 1 week, the dose is increased per the discretion of the Investigator to a cumulative dose of 2 to 2.5 g/day of MMF no later than by Week 4 (Day 28). The dose can be administered in multiple divided doses. Participants continue to receive 2 to 2.5 g/day of MMF for a minimum duration of 50 weeks after which it may be decreased or discontinued based on the Investigators' judgment and the KDIGO clinical practice guidelines (KDIGO Clinical practice guideline for glomerulonephritis. Kidney International Supplements. 2012; 2(2):140).

For participants who have initiated corticosteroid induction treatment prior to Screening and do not meet Exclusion Criterion:
1. If the participant already received methylprednisolone IV≥1 g or equivalent and is receiving MMF≥2 g/day prior to Screening, then methylprednisolone IV is not given and MMF w is continued at the current dose for a minimum duration of 50 weeks, after which it may be decreased or discontinued based on the Investigator's judgment and the KDIGO clinical practice guidelines.
2. If the participant already received methylprednisolone IV≥1 g or equivalent and is receiving MMF<2 g/day, then methylprednisolone IV is not given and the MMF dose is increased during the Screening Period (no later than Day 1) to a cumulative dose of 1 to 1.5 g/day. Participants continue to receive 1 to 1.5 g/day for 1 week after which the MMF dose is increased per the discretion of the Investigator to 2 to 2.5 g/day to be achieved no later than Week 4 (Day 28). These doses can be administered in multiple divided doses. Participants continue to receive 2 to 2.5 g/day for a minimum duration of 50 weeks, after which it may be decreased or discontinued based on the Investigator's judgment and the KDIGO clinical practice guidelines.
3. If a participant is already receiving prednisone or prednisone equivalent, the dose is continued until Day 2 at which time 0.5 mg/kg/day should be administered (the minimum and maximum dose allowed are 30 mg/day and 60 mg/day, respectively). The prednisone dose is tapered starting on Week 2 (Day 14) according to the schedule in Table.

TABLE 4
Corticosteroid Taper for Participants With Lupus Nephritis

| | Prednisone or Equivalent Dose | | | |
|---|---|---|---|---|
| | (0.5 mg/kg/day)^{a} According to Baseline | | | |
| Study | Body Weight | | | |
| week | 40 to 60 kg | 61 to 80 kg | 81 to 100 kg | >101 kg |
| Screening to | 30 | 40 | 50 | 60 |
| Week 2 | | | | |
| 2 | 25 | 35 | 40 | 50 |
| 4 | 25 | 30 | 30 | 40 |
| 6 | 20 | 25 | 20 | 30 |
| 8 | 15 | 20 | 15 | 20 |
| 10 | 10 | 15 | 10 | 10 |
| 12 onward | 7.5 | 7.5 | 7.5 | 7.5 |

^{a}The minimum weight for adult doses of ravulizumab is 40 kg. The minimum and maximum starting doses of prednisone are 30 mg and 60 mg, respectively.

Other considerations regarding MMF dosing:
1. An equivalent dose of enteric-coated mycophenolic acid sodium (MPS) can be used instead of MMF (i.e., 360 mg dose MPS is equivalent to a 500 mg dose of MMF)
2. Investigators can adjust the dosage of MMF due to tolerance or AEs. After the symptoms resolve, the Investigator can attempt to increase MMF (or equivalent) to the goal level. If symptoms return, then the participant is continued on the highest tolerable dose.
3. Any changes to the dose of MMF and the justification are documented in the CRF.

Other considerations regarding the corticosteroid taper:
1. All participants have a scheduled corticosteroid taper starting on Day 14. Participants reduce their prednisone dose according to their baseline body weight over 10 weeks until the dose is 7.5 mg/day by Week 12 (Table 4).
2. Deviations from the scheduled corticosteroid taper for any reason other than Renal Flare or Extrarenal SLE Flare confound interpretation, so every attempt should be made to adhere to the tapering schedule.
3. If disease is too clinically active in the opinion of the Investigator to begin the corticosteroid taper after Week 2, then the participant can continue to receive his or her initial corticosteroid dose for up to an additional 28 days. Similarly, participants who have started the taper and whose disease is too clinically active to continue tapering, can remain at the same taper dose achieved for up to an additional 28 days. Failure to achieve the corticosteroid taper by Week 12 is not be considered as Treatment Failure and is captured as a secondary endpoint.
4. However, the prednisone dose can NOT be increased beyond the taper dose achieved unless participant meets the protocol-defined criteria for Renal Flare and/or Severe Extrarenal SLE Flare in which case these participants receive Rescue Therapy and are included as Treatment Failures.

6. Background Therapy for IgAN Cohort

The background therapies for participants in the IgAN cohort is consistent with standard of care and include the maximumly tolerated dose of RAS-blocking agents, such as ACE inhibitors or ARBs. The background treatment is held stable throughout the Treatment Period of the study.

7. Rescue Therapy for LN Cohort

Participants in the LN cohort receive Rescue Therapy in the event of a protocol-defined Renal Flare or Severe Extrarenal SLE Flare. Rescue Therapy is defined as intensification of current standard of care or introduction of new immunosuppressive therapies.

The specific choice of Rescue Therapy(ies) is generally at the discretion of the Investigator. However, the following guidelines for corticosteroid dosing for protocol-defined Renal Flare and Severe Extrarenal SLE Flares should be considered to maintain treatment consistency:
1. Participants with protocol-defined Renal Flare can be treated with prednisone up to 0.5 mg/kg/day (not to exceed 60 mg/day) for up to 2 weeks. Prednisone can then be tapered weekly to 10 mg/day within 6 weeks after the initial prednisone increase. Prednisone can further be tapered to 7.5 mg/day at the discretion of the Investigator.
2. Participants with Severe Extrarenal SLE flare can be treated with prednisone up to 1 mg/kg/day (not to exceed 60 mg/day) for up to 2 weeks. Prednisone is then be tapered every 2 weeks to achieve 7.5 mg/day within 12 weeks after the initial corticosteroid increase.
3. Intravenous corticosteroids in equivalent doses can be allowed if gastrointestinal involvement temporality precludes oral corticosteroid use.

Prednisone≥10 mg for ≤14 days will not be considered Rescue Therapy in the following instances: (1) renal flares not meeting the protocol defined criteria for Renal Flare, (2) extrarenal SLE flares not requiring >14 days of >10 mg prednisone or equivalent or introduction of new immunosuppressive medication in the opinion of Investigator; and (3) other medical conditions or surgery.

8. Concomitant Therapy

Any medication or therapy (including over-the-counter or prescription medicines, vaccines, vitamins, and/or herbal supplements) deemed necessary for the participant's care during the study, or for the treatment of any AE, along with any other medications, other than those listed as disallowed medications, can be given at the discretion of the Investigator.

If adequate blood pressure control is not achieved during the study, participants can receive additional antihypertensive agents, but not agents that affect proteinuria during the study. It is recommended that NSAIDs not be initiated during the study due to the possibility of adverse effects on renal function. They may be used, however, if necessary for the control of symptoms.

For participants in the LN cohort: (1) pneumocystis pneumonia prophylaxis is allowed at the discretion of the Investigator, (2) treatment with antimalarial agents such as hydroxychloroquine are allowed unless contraindicated, and (3) measures to prevent and treat osteoporosis are strongly encouraged during the study; these measures may include any, or all, of the following: calcium carbonate or citrate, Vitamin D, and bisphosphonates.

Participants in both cohorts are prohibited from receiving any of the following medications and therapies during the entire duration of study participation: (1) experimental interventions or therapies, (2) eculizumab, and (3) SGLT-2 inhibitors and direct renin antagonists.

In the event that a participant receives a prohibited medication and/or therapy, the participant should discontinue study drug with the exception of SGLT-2 inhibitors and direct renin antagonists (SGLT-2 inhibitors and direct renin antagonists are prohibited but may not require discontinuation of study drug based on the discussion and approval of the Investigator and Medical Monitor).

Participants in the IgAN cohort are also prohibited from receiving any of the following medications and therapies during the entire duration of study participation: (1) hydroxychloroquine, (2) immunosuppressive agents (e.g., MMF), and (3) systemic corticosteroids for >14 consecutive days (short-term steroid course for ≤14 days for medical conditions not related to IgAN or surgery are permitted).

### Example 2: Further Details of Phase 2, Double-Blind, Randomized, Placebo-Controlled Study to Evaluate the Efficacy and Safety of Ravulizumab in Adult Participants With Proliferative Lupus Nephritis (LN) or Immunoglobulin A Nephropathy (IgAN)

A Phase 2, randomized, double-blind, placebo-controlled, multicenter study (referred to as “Study ALXN1210 NEPH 202”) of ravulizumab in addition to background therapy consistent with the standard of care is conducted in 120 adult participants (18 to 75 years of age) with either LN or IgAN. The study design for the LN cohort is set forth in FIG. 1. The study design for the IgAN cohort is set forth in FIG. 2.

1. Overall Design

Study ALXN1210 NEPH 202 is a Phase 2, randomized, double-blind, placebo-controlled, multicenter study of ravulizumab in addition to background therapy consistent with the standard of care in 120 adult participants (18 to 75 years of age) with either LN or IgAN. All participants are naive to complement inhibitor treatment and have either a diagnosis of LN with an active flare or IgAN based on kidney biopsy, estimated glomerular filtration rate (eGFR)≥30 mL/min/1.73m2, and proteinuria [defined as urine protein to creatinine ratio (UPCR)≥1 g/g from one 24-hr urine collection (LN cohort) or as mean protein≥1 g/24-hr from 2 valid 24-hr collections (IgAN cohort)]. Participants in the IgAN cohort have been treated with stable doses of the maximum tolerated renin-angiotensin system (RAS) inhibiting medications and have controlled, stable blood pressure (<140/90 mmHg) for ≥3 months prior to Screening.

Approximately 60 participants in each disease cohort are randomly assigned in a 2:1 ratio to receive ravulizumab or placebo (40 ravulizumab, 20 placebo). Randomization is stratified by whether corticosteroid induction treatment was initiated prior to Screening versus during the Screening Period for participants in the LN cohort and by mean proteinuria (1 to 2 g/day versus >2 g/day) from 2 valid 24-hr urine collections during Screening Period for participants in the IgAN cohort.

The study consists of an up to 6-week Screening Period, a 26-week Initial Evaluation Period, a 24-week Extension Period, and a 36 week post-treatment Follow-up Period.

During the Initial Evaluation Period, all participants receive a weight based loading dose of ravulizumab or placebo on Day 1, followed by maintenance doses of ravulizumab or placebo on Day 15 and then once every 8 weeks (q8w) thereafter. Loading and maintenance doses will be determined based on body weight, as set forth in Table 2. All participants receive background therapy consistent with the standard of care for participants with LN and IgAN throughout the study.

During the 24-week Extension Period, participants in the LN cohort continue to receive their randomized allocation of study drug (ravulizumab or placebo) q8w. For the IgAN cohort, participants in the placebo group receive a blinded loading dose of ravulizumab at Week 26 and participants in the ravulizumab group receive a blinded ravulizumab dose of 900 mg at Week 26. Starting Week 28, all participants in the IgAN cohort receive open-label weight based doses of ravulizumab q8w until the end of the Extension Period.

During the 36-week Post treatment Follow-up Period, all participants continue to receive standard of care and are monitored for safety, clinical events of interest, and kidney function. All participants, including participants that discontinue the study drug early, are followed for safety until 8 weeks after the last dose of study drug. The end of study is defined as the last participant's last visit in the Post-treatment Follow-up Period.

To ensure the adequacy of the dose regimen, an interim pharmacokinetics (PK)/pharmacodynamic (PD) analysis for dose confirmation is conducted by an independent clinical pharmacologist. The interim PK is conducted using masked PK/PD data from the first participants treated with ravulizumab (a minimum of 3 participants in each disease-specific cohort). In the event of dose adjustments, the participants treated with the previous dose switch over to the new dose and continue treatment on study, but are excluded from the primary efficacy analysis. Replacement participants can be enrolled to preserve study power.

Disclosure Statement: This is a parallel group treatment study with 2 disease cohorts of participants randomly assigned to 1 of 2 treatments that are participant, Investigator, and outcomes assessor blinded.

Number of Participants: Approximately 120 adult participants are randomized. This includes approximately 60 participants in the LN cohort and approximately 60 participants in the IgAN cohort.

Eligible participants are enrolled into the study and are randomized in a 2:1 ratio to receive either ravulizumab IV infusion or placebo IV infusion in combination with background therapy.

Ravulizumab is supplied as a sterile, preservative free 10 mg/mL solution in single use vials, designed for administration via IV infusion by diluting into commercially available saline (0.9% sodium chloride injection). Dosages are based on the participant's body weight, as shown in Table 2.

2. Schedule of Activities

The Schedule of Activities for the Initial Evaluation Period (Screening to Week 26 (Day 183) Visit) are set forth in Table 5 for the LN cohort and Table 6 for the IgAN cohort/The Schedule of Activities for the Extension Period are set forth in Table 7 for the LN cohort and in Table 8 for the IgAN cohort.

The Schedule of Activities for the Post-Treatment Follow-up for both cohorts is set forth in Table 9.

TABLE 5
Schedule of Activities During the Initial Evaluation Period:
Screening to Week 26 Visit (LN Cohort)

| | Screen- | | | | | | | | | | | | |
|---|---|---|---|---|---|---|---|---|---|---|---|---|---|
| | ing | Initial Evaluation Period | | | | | | | | | | | |
| | Visit | | | | | | | | | | | Eval. | Notes |
| | 1 | 2 | 3 | 4 | 5 | 6 | 7 | 8 | 9 | 10 | 11 | for | Additional visits can |
| | Week | | | | | | | | | | | Renal | be performed as |
| | Up to | | | | | | | | | | | Flare | needed. An ED Visit |
| | 6 W | | W1 | W2 | W4 | W6 | W10 | W14 | W18 | W22 | W26/ED^{a} | and | is required if |
| | Days and Window | | | | | | | | | | | Extrarenal | participants |
| | D-42 | | D8 ± | D15 ± | D29 ± | D43 ± | D71 ± | D99 ± | D127 ± | D155 ± | D183 ± | SLE | discontinue study |
| Period | to-1 | D 1 | 2 | 3 | 3 | 5 | 5 | 5 | 5 | 5 | 5 | Flare | drug early. |

General Assessments/Procedures

| Informed | X | | | | | | | | | | | |
|---|---|---|---|---|---|---|---|---|---|---|---|---|
| consent | | | | | | | | | | | | |
| Inclusion/ | X | | | | | | | | | | | Confirm eligibility |
| exclusion | | | | | | | | | | | | prior to first dose of |
| | | | | | | | | | | | | ravulizumab; |
| | | | | | | | | | | | | participants may be |
| | | | | | | | | | | | | rescreened once |
| Demographics | X | | | | | | | | | | | |
| Medical hisotry | X | | | | | | | | | | | |
| LN | X | | | | | | | | | | | LN guidelines |
| history/ | | | | | | | | | | | | |
| diagnosis | | | | | | | | | | | | |
| Documentation | X | | | | | | | | | | | Biopsy obtained ≤6 |
| of kidney | | | | | | | | | | | | months prior to |
| biospy | | | | | | | | | | | | Screening or during |
| | | | | | | | | | | | | Screening. Send local |
| | | | | | | | | | | | | pathology report and |
| | | | | | | | | | | | | slides to Central |
| | | | | | | | | | | | | Pathology laboratory |
| Meningococcal, | X^{b} | | | Completion of vaccination series according to | | | | | | | | |
| Hib and | | | | national and local schedule guidelines | | | | | | | | |
| S pneumoniae | | | | | | | | | | | | |
| vaccination | | | | | | | | | | | | |
| Prior LN | X | | | | | | | | | | | Record corticosteroid |
| therapy | | | | | | | | | | | | and MMF usage |
| Weight^{b} | X | X | X | X | X | X | X | X | X | X | X | |
| Height | X | | | | | | | | | | | |
| Pregnancy test | X | X | | X | | | X | | X | | X | Serum test required at |
| (WOCBP only) | | | | | | | | | | | | Screening and ED; |
| | | | | | | | | | | | | urine test all other |
| | | | | | | | | | | | | visits |
| HIV, HCV, and | X | | | | | | | | | | | |
| HBV | | | | | | | | | | | | |
| Dispense | X | | | | | | | | | | | Instruct participants to |
| participant | | | | | | | | | | | | carry safety card at all |
| safety card | | | | | | | | | | | | times and bring it to |
| | | | | | | | | | | | | scheduled visits |

Efficiacy Assessments

| 24-hr urine | X | | | | | | | | | | X | X | One collection is |
|---|---|---|---|---|---|---|---|---|---|---|---|---|---|
| collection^{c} | | | | | | | | | | | | | needed as soon as |
| | | | | | | | | | | | | | possible during |
| | | | | | | | | | | | | | Screening. |
| | | | | | | | | | | | | | Two collections must |
| | | | | | | | | | | | | | be obtained within |
| | | | | | | | | | | | | | 2 weeks prior to |
| | | | | | | | | | | | | | Week 26 |
| Morning spot | X | X | X | X | X | X | X | X | X^{e} | X | X | X | Obtain sample prior to |
| urine sample^{c} | | | | | | | | | | | | | dosing, vaccination, |
| | | | | | | | | | | | | | and biopsy |
| eGFR | X | X | X | X | X | X | X | X | X | X | X | X^{d} | One blood draw for |
| | | | | | | | | | | | | | eGFR (serum |
| | | | | | | | | | | | | | creatinine) is |
| | | | | | | | | | | | | | requried within |
| | | | | | | | | | | | | | 2 weeks prior to the |
| | | | | | | | | | | | | | Week 26 visit. |
| Monitor for | | Continuous monitoring | | | | | | | | | | X | Document use of |
| Renal Flare | | | | | | | | | | | | | Rescue Therapy |
| and/or | | | | | | | | | | | | | and/or repeat biopsy^{d}, |
| Extrarenal SLE | | | | | | | | | | | | | if applicable. |
| Flare^{d} | | | | | | | | | | | | | |
| Blood sample | X | X | | X | | | X | | X | | X | X | |
| for C3, C4, and | | | | | | | | | | | | | |
| CH50 | | | | | | | | | | | | | |

Safety Assessments

| Physical | X | X | | | | | | | | | X | | |
|---|---|---|---|---|---|---|---|---|---|---|---|---|---|
| examination | | | | | | | | | | | | | |
| Abbreviated PE | | | X | X | X | X | X | X | X | X | | | |
| Vital signs^{f} | X | X | X | X | X | X | X | X | X | X | X | | |
| ECG^{g} | X | | | | | | | | | | X | | |
| Prior | X | | | | | | | | | | | | |
| medications | | | | | | | | | | | | | |
| and procedures | | | | | | | | | | | | | |
| Concomitant | | Continuous monitoring | | | | | | | | | | X | |
| medications, | | | | | | | | | | | | | |
| nonpharmacologic | | | | | | | | | | | | | |
| therapies, | | | | | | | | | | | | | |
| and procedures | | | | | | | | | | | | | |
| Adverse events | Continuous monitoring | | | | | | | | | | | X | |
| Clinical | X | X | X | X | X | X | X | X | X | X | X | X^{d} | |
| chemistry | | | | | | | | | | | | | |
| Hematology | X | X | X | X | X | X | X | X | X | X | X | | |
| and coagulation | | | | | | | | | | | | | |
| Urinalysis and | X | X | X | X | X | X | X | X | X | X | X | X | Obtain sample from a |
| sediment | | | | | | | | | | | | | morning void prior to |
| | | | | | | | | | | | | | dosing |
| Participant | | X | X | X | X | X | X | X | X | X | X | X | Confirm participants |
| safety card | | | | | | | | | | | | | carry safety card at all |
| review | | | | | | | | | | | | | times |

Pharmacokinetic and Pharmacodynamic Assessments

| | | | | | | | | | |
|---|---|---|---|---|---|---|---|---|---|
| Blood samples | B/P | X | T/P | X | T/P | T/P | T | X | Samples can be |
| for PK/PD^{h} | | | | | | | | | obtained anytime at |
| | | | | | | | | | ED Visit |
| Blood samples | B | | | X | T | | T | | Samples can be |
| for ADA | | | | | | | | | obtained anytime at |
| | | | | | | | | | ED Visit |

Exploratory Assessments

| | | | | | | | | |
|---|---|---|---|---|---|---|---|---|
| EQ-5D-5L | | X | | X | | X | | |
| SF-36 | | X | | X | | X | | |
| FACIT-Fatigue | | X | | | | X | | |
| SLEDAI-2K | | X | | | | X | X | Perform as needed for |
| | | | | | | | | evaluation of |
| | | | | | | | | Extrarenal SLE Flare |
| Blood and | X | B | | | | X | X | |
| urine samples | | | | | | | | |
| for biomarkers | | | | | | | | |
| Blood samole | X | B | | | | X | X | |
| for anti-dsDNA | | | | | | | | |
| and anti-C1q | | | | | | | | |
| Blood and | X | X | X | X | X | X | | To be performed at |
| urine samples | | | | | | | | selected sites only. |
| for RTCA | | | | | | | | |
| Kidney biopsyⁱ | | | | | | | Xⁱ | Send local pathology |
| | | | | | | | | report and microscopy |
| | | | | | | | | slides to the Central |
| | | | | | | | | Pathology Laboratory |

Administration of Study Intervention

| Background | X | Continuous monitoring | | | | | |
|---|---|---|---|---|---|---|---|
| LN therapy | | | | | | | |
| Ravulizumab or | | X | X | X | X | —^{j} | Administer after all |
| placebo | | | | | | | other required |
| | | | | | | | tests/procedures |

Note:
All assessments are performed prior to administration of study drug on dosing days, unless otherwise specified.
^{a}For participants who discontinue study drug prior to the end of the Initial Evaluation Period, the ED visit is completed as soon as possible. In addition, a Follow-up Phone Call is performed 8 weeks following the participant's last dose of study drug to collect information on concomitant medications, nonpharmacologic therapies and procedures, and AEs.
^{b}Weight is obtained at every visit and measured predose on dosing visits. The dose regimen is based on the last recorded study visit body weight. If the study drug is prepared the night before a visit, the weight from the most recent study visit is used.
^{c} The 24-hr urine collection and spot urine samples are obtained prior to or >7 days after administration of vaccine(s) and biopsy procedures.
^{d}Renal Flare and/or Severe Extrarenal SLE Flare can occur at any time through Week 50. Evaluation of Renal Flare requires a UPCR from a spot urine sample that is confirmed on a 24-hr urine collection as well as 2 serum creatinine samples obtained with a 2-week period. Evaluation of Renal and Extrarenal SLE Flare is performed as soon as possible upon notification to the Investigator of symptom onset. If Renal Flare or Extrarenal SLE Flare occurs between scheduled visits, only the assessments for the Renal Flare/Extrarenal SLE Flare visit are needed. If Renal Flare or Extrarenal SLE Flare occur on a scheduled visit, all scheduled assessments are performed for that visit as well as any additional assessments required for the evaluation of the flare.
^{e}Two spot urine samples are obtained the same morning at Week 18.
^{f}Vital sign measurements include systolic and diastolic BP, pulse oximetry, heart rate, respiratory rate, and temperature. On dosing days, vital signs are taken predose.
^{g}Single 12-lead ECG is collected at Screening and predose on Day 183 and any time on the ED visit day. Participants are supine for approximately 5 to 10 minutes before ECG collection and remain supine but awake during ECG collection.
^{h}For indicated visits falling on dosing days, PK/PD samples are collected predose (within 0.5 hours prior to the start of infusion) and at EOI (within 0.5 hours after the EOI from the participant's opposite, noninfused arm). In order to minimize needle sticks to the participant, the predose sample may be drawn through the venous access created for the dose infusion, prior to administration of the dose. As noted, the postdose sample must be drawn from the opposite, noninfused arm. For indicated visits not falling on dosing days, samples may be collected at any time that visit day.
ⁱParticipants can receive a kidney biopsy for clinical reasons or for evaluation of a Renal Flare at the discretion of the Investigator. The local pathology report and microsection slides from kidney biopsies performed at other times during the study prior to Week 86 is sent to the Central Pathology Laboratory for review as soon as possible. Since examination of the biopsy pathology results can potentially unblind the study treatment (ravulizumab or placebo), Investigators and study site personnel do not examine the biopsy pathology results for immunohistochemistry of complement prior to Week 50.
^{j}The primary efficacy endpoint assessment is obtained prior to dosing on Day 183. Dosing on Day 183 is the start of the Extension Period.
Abbreviations: ADA = antidrug antibody;
AE = adverse event;
B = baseline;
BP = blood pressure;
C3, C4, C1q = complement component 3, 4, and C1q;
CH50 = 50% hemolytic complement activity;
D = day;
dsDNA = double-stranded DNA;
ED = early discontinuation;
eGFR = estimated glomerular filtration rate;
EOI = end of infusion;
EQ-5D-5L = European Quality of Life Health 5-item questionnaire dimensions 5 level;
Eval. = evaluation;
FACIT = Functional Assessment of Chronic Illness Therapy;
HBV = hepatitis B virus;
HCV = hepatitis C virus;
Hib = Haemophilus influenzae type b;
IgAN = immunoglobulin A nephropathy;
LN = lupus nephritis;
MMF = mycophenolate mofetil;
P = postdose;
PD = pharmacodynamics;
PE = physical examination;
PK = pharmacokinetics;
RTCA = real time complement activity;
SLEDAI-2K = Systemic Lupus Erythematosus Disease Activity Index Safety of Estrogen in Lupus Erythematosus National Assessment (SELENA) Modification;
SF-36 = Short Form (36) Health Survey;
SLE = systemic lupus erythematosus;
T = trough (predose);
UPCR = urine protein to creatinine ratio;
W = week;
WOCBP = women of childbearing potential.

TABLE 6
Schedule of Activities During the Initial Evaluation Period: Screening to Week 26 Visit (IgAN Cohort)

| | Screen- | | | | | | | | | | |
|---|---|---|---|---|---|---|---|---|---|---|---|
| | ing | Initial Evaluation Period | | | | | | | | | |
| | Visit | | | | | | | | | | |
| | 1 | 2 | 3 | 4 | 5 | 6 | 7 | 8 | 9 | 10 | |
| | Week | | | | | | | | | | Notes |
| | Up to | | | | | | | | | | Additional visits are |
| | 6 W | | W1^{a} | W2 | W4 | W10 | W14 | W18 | W22 | W26/ED^{b} | performed as needed. |
| | Days and Window | | | | | | | | | | An ED Visit is required if |
| | D-42 | | D8 ± | D15 ± | D29 ± | D71 ± | D99 ± | D127 ± | D155 ± | D183 ± | participants discontinue |
| Period | to-1 | D 1 | 2 | 3 | 3 | 3 | 3 | 3 | 3 | 3 | early. |

General Assessments/Procedures

| | | | | | | | | | | | |
|---|---|---|---|---|---|---|---|---|---|---|---|
| Informed | X | | | | | | | | | | |
| consent | | | | | | | | | | | |
| Inclusion/ | X | | | | | | | | | | Confirm eligibility |
| exclusion | | | | | | | | | | | prior to first dose of |
| | | | | | | | | | | | ravulizumab; |
| | | | | | | | | | | | participants can be |
| | | | | | | | | | | | rescreened once |
| Demographics | X | | | | | | | | | | |
| Medical hisotry | X | | | | | | | | | | |
| IgAN | X | | | | | | | | | | |
| history/ | | | | | | | | | | | |
| diagnosis | | | | | | | | | | | |
| Documentation | X | | | | | | | | | | Kidney biopsy performed |
| of kidney | | | | | | | | | | | during or prior to Screening |
| biospy | | | | | | | | | | | (any time prior to Day 1). |
| | | | | | | | | | | | Send local pathology report |
| | | | | | | | | | | | and microscopy slides to |
| | | | | | | | | | | | Central Pathology |
| | | | | | | | | | | | Laboratory |
| Prior IgAN | X | | | | | | | | | | Record ACE/ARB usage |
| therapy | | | | | | | | | | | |
| Meningococcal, | X^{c} | | | | | | | | | | |
| Hib and | | | | | | | | | | | |
| S pneumoniae | | | | | | | | | | | |
| vaccination | | | | | | | | | | | |
| Weight^{d} | X | X | X | X | X | X | X | X | X | X | |
| Height | X | | | | | | | | | | |
| HIV, HCV, and | X | | | | | | | | | | |
| HBV | | | | | | | | | | | |
| Pregnancy test | X | X | | X | | X | | X | | X | Serum test require at |
| (WOCBP only) | | | | | | | | | | | Screening and ED; urine test |
| | | | | | | | | | | | all other visits |
| Dispense | X | | | | | | | | | | Instruct participants to carry |
| participant | | | | | | | | | | | safety card at all times and |
| safety card | | | | | | | | | | | bring it to scheduled visits |

Efficiacy Assessments

| 24-hr urine | X | | | | | | | | | X | 2 valid 24-hour collections |
|---|---|---|---|---|---|---|---|---|---|---|---|
| collection^{c} | | | | | | | | | | | are required during |
| | | | | | | | | | | | Screening and within 2 |
| | | | | | | | | | | | weeks of the Week 26 Visit |
| Morning spot | X | X | X | X | X | X | X | X^{e} | X | X | Obtain sample prior to |
| urine sample^{c} | | | | | | | | | | | dosing |
| eGFR | X | X | X | X | X | X | X | X | X | X | |
| Blood sample | X | X | | X | | X | | X | | X | |
| for C3, C4, and | | | | | | | | | | | |
| CH50 | | | | | | | | | | | |
| | Screen- | | | | | | | | | | |
| | ing | Initial Evaluation Period | | | | | | | | | |
| | Visit | | | | | | | | | | |
| | 1 | 2 | 3 | 4 | 5 | 6 | 7 | 8 | 9 | 10 | |
| | Week | | | | | | | | | | Notes |
| | Up to | | | | | | | | | | Additional visits can be |
| | 6 W | | W1^{a} | W2 | W4 | W10 | W14 | W18 | W22 | W26/ED^{b} | performed as needed. An |
| | Days and Window | | | | | | | | | | ED Visit is required if |
| | D-42 | | D8 ± | D15 ± | D29 ± | D71 ± | D99 ± | D127 ± | D155 ± | D183 ± | participants discontinue |
| Period | to-1 | D 1 | 2 | 3 | 3 | 3 | 3 | 3 | 3 | 3 | early |

Safety Assessments

| Physical | X | X | | | | | | | | X | |
|---|---|---|---|---|---|---|---|---|---|---|---|
| examination | | | | | | | | | | | |
| Abbreviated PE | | | X | X | X | X | X | X | X | | |
| Vital signs^{f} | X | X | X | X | X | X | X | X | X | X | |
| ECG^{g} | X | | | | | | | | | X | |
| Prior | X | | | | | | | | | | |
| medications and | | | | | | | | | | | |
| procedures | | | | | | | | | | | |
| Concomitant | Continuous monitoring | | | | | | | | | | |
| medications, | | | | | | | | | | | |
| nonpharmacologic | | | | | | | | | | | |
| therapies, and | | | | | | | | | | | |
| procedures | | | | | | | | | | | |
| Adverse events | Continuous monitoring | | | | | | | | | | |
| Clinical | X | X | X | X | X | X | X | X | X | X | |
| chemistry | | | | | | | | | | | |
| Hematology and | X | X | X | X | X | X | X | X | X | X | |
| coagulation | | | | | | | | | | | |
| Urinalysis and | X^{h} | X | X | X | X | X | X | X | X | X | Obtain sample from a |
| sediment | | | | | | | | | | | morning void prior to dosing |
| Participant | | X | X | X | X | X | X | X | X | X | Confirm participants carry |
| safety card | | | | | | | | | | | safety card at all times |
| review | | | | | | | | | | | |

Pharmacokinetic and Pharmacodynamic Assessments

| | | | | | | | | |
|---|---|---|---|---|---|---|---|---|
| Blood samples | B/P | X | T/P | X | T/P | T/P | T | At ED visit, samples can be |
| for PK/PDⁱ | | | | | | | | anytime. |
| Blood samples | B | | | X | T | | T | At ED visit, samples can be |
| for ADA | | | | | | | | anytime. |

Exploratory Assessments

| EQ-5D-5L | | X | | | | X | | | | X | |
|---|---|---|---|---|---|---|---|---|---|---|---|
| SF-36 | | X | | | | X | | | | X | |
| Blood and urine | X | X | | X | | X | | X | | X | |
| samples for | | | | | | | | | | | |
| biomarkers | | | | | | | | | | | |
| Blood and urine | X | X | | X | | X | | X | | X | To be performed at selected |
| samples for | | | | | | | | | | | sites only. |
| RTCA | | | | | | | | | | | |
| | Screen- | | | | | | | | | | |
| | ing | Initial Evaluation Period | | | | | | | | | |
| | Visit | | | | | | | | | | |
| | 1 | 2 | 3 | 4 | 5 | 6 | 7 | 8 | 9 | 10 | |
| | Week | | | | | | | | | | Notes |
| | Up to | | | | | | | | | | Additional visits are |
| | 6 W | | W1^{a} | W2 | W4 | W10 | W14 | W18 | W22 | W26/ED^{b} | performed as needed. An |
| | Days and Window | | | | | | | | | | ED Visit is required if |
| | D-42 | | D8 ± | D15 ± | D29 ± | D71 ± | D99 ± | D127 ± | D155 ± | D183 ± | participants discontinue |
| Period | to-1 | D 1 | 2 | 3 | 3 | 3 | 3 | 3 | 3 | 3 | early |
| Kidney biopsy | | Continuous monitoring | | | | | | | | | Send the site pathology report |
| (if indicated per | | | | | | | | | | | and microscopy slides to the |
| Investigator)ⁱ | | | | | | | | | | | Central Pathology Laboratory |

Administration of Study Intervention

| Randomization | | X | | | | | |
|---|---|---|---|---|---|---|---|
| Background | X | Continuous monitoring | | | | | |
| IgAN therapy | | | | | | | |
| Ravulizumab or | | X | X | X | X | —^{k} | Administer after all |
| placebo | | | | | | | other required |
| | | | | | | | tests/procedures |

^{a}The Week 1 visit is not required for participants enrolled after completion of the Dose Confirmation Analysis.
^{b}For participants who discontinue the study prior to the end of the Initial Evaluation Period, the ED visit should be completed as soon as possible. In addition, a Follow-up Phone Call is performed 8 weeks following the participant's last dose of study drug to collect information on concomitant medications, nonpharmacological therapies and procedures, and AEs.
^{c}The 24-hr urine collection and spot urine samples must be obtained prior to or >7 days after administration of vaccine(s) and biopsy procedures.
^{d}Weight should be obtained at every visit and measured predose on dosing visits. The dose regimen is based on the last recorded study visit body weight. If the study drug is prepared the night before a visit, the weight from the most recent study visit is used.
^{e}Two spot urine samples re obtained the same morning at Week 18.
^{f}Vital sign measurements include systolic and diastolic BP, pulse oximetry, heart rate, respiratory rate, and temperature. On dosing days, vital signs are taken predose.
^{g}Single 12-lead ECG is collected at Screening and predose on Day 183 and any time on the ED visit day. Participants are supine for approximately 5 to 10 minutes before ECG collection and remain supine but awake during ECG collection.
^{h}For participants in the IgAN cohort, eligibility for hematuria can be determined via the local laboratory.
ⁱThe PKPD samples are collected predose (within 0.5 hours prior to the start of infusion) and at EOI (within 0.5 hours after the EOI from the participant's opposite, noninfused arm). In order to minimize needle sticks to the participant, the predose sample may be drawn through the venous access created for the dose infusion, prior to administration of the dose. As noted, the postdose sample must be drawn from the opposite, noninfused arm. For indicated visits not falling on dosing days, samples may be collected at any time that visit day.
^{j}In the event that a participant has a kidney biopsy (performed at the discretion of the Investigator for clinical reasons as part of standard of care), the local pathology report and microscopy slides should be sent to the Central Pathology Laboratory as soon as possible. Because examination of the kidney biopsy pathology may potentially unblind the study treatment (ravulizumab or placebo), Investigators and study site personnel should not examine the biopsy pathology for immunohistochemistry of complement prior to Week 50.
^{k}The primary efficacy endpoint assessment is obtained prior to dosing on Day 183. Dosing on Day 183 is the start of the Extension Period.
Abbreviations: ADA = antidrug antibody;
ACE = angiotensin-converting enzyme;
AE = adverse event;
ARB = angiotensin II receptor blocker;
B = baseline;
BP = blood pressure;
C3, C4 = complement component 3 and 4;
CH50 = 50% hemolytic complement activity;
D = day;
ED = early discontinuation;
eGFR = estimated glomerular filtration rate;
EOI = end of infusion;
EQ-5D-5L = European Quality of Life Health 5-item questionnaire dimensions 5 level;
FACIT = Functional Assessment of Chronic Illness Therapy;
HBV = hepatitis B virus;
HCV = hepatitis C virus;
Hib = Haemophilus influenzae type b;
IgAN = immunoglobulin A nephropathy;
LN = lupus nephritis;
MMF = mycophenolate mofetil;
P = postdose;
PD = pharmacodynamics;
PE = physical examination;
PK = pharmacokinetics;
RTCA = real time complement activity;
SLEDAI-2K = Systemic Lupus Erythematosus Disease Activity Index Safety of Estrogen in Lupus Erythematosus National Assessment (SELENA) Modification;
SF-36 = Short Form (36) Health Survey;
SLE = systemic lupus erythematosus;
T = trough (predose);
UPCR = urine protein to creatinine ratio;
W = week;
WOCBP = women of childbearing potential.

TABLE 7
Schedule of Activities During the Extension Period: Week 26
Dose Administration, Week 34 to Week 50 Visits (LN Cohort)

| | Extension Period | | | | | |
|---|---|---|---|---|---|---|
| | Visit | | | | | |
| | 12 | 13 | 14 | 15 | | |
| | Week | | | | | Notes |
| | W26 | W34 | W42 | W50/ED^{a} | Eval. for Renal | Additional visits are performed |
| | Days and Window | | | | Flare and | as needed. An ED Visit is |
| | | D239 ± | D295 ± | D351 ± | Extrarenal | performed if participants |
| Period | D183 | 7 | 7 | 7 | SLE Flare | discontinue early. |

General Assessments/Procedures

| | | | | |
|---|---|---|---|---|
| Weight^{b} | X | X | X | |
| Pregnancy test | X | X | X | Serum pregnancy test required at |
| (WOCBP only) | | | | ED; urine pregnancy test all other |
| | | | | visits. |

Efficacy Assessments

| 24-hr urine Collection | | | | X | X | Two 24-hr urine collections required |
|---|---|---|---|---|---|---|
| | | | | | | within 2 weeks prior to the Week |
| | | | | | | 50 Visit. Renal Flare requires a 24-hr |
| | | | | | | urine for confirmation^{c} |
| Morning spot urine sample | | X | X | X | X | Obtain predose on dosing visits |
| eGRF | | X | X | X | X^{h} | One blood draw for eGFR (serum |
| | | | | | | creatinine) is required within |
| | | | | | | 2 weeks prior to the Week 50 visit. |
| Monitor for RenalFlare | Continuous monitoring | | | | X | Document use of Rescue Therapy |
| and/or Extrarenal SLE | | | | | | and/or repeat biopsy^{f} if applicable |
| Flare^{c} | | | | | | |
| Blood sample for C3, C4, | | X | X | X | X | |
| and CH50 | | | | | | |

Safety Assessments

| Physical examination | | | | X | | |
|---|---|---|---|---|---|---|
| Abbreviated physical | | X | X | | | |
| examination | | | | | | |
| Vital signs^{d} | | X | X | X | | |
| ECG^{e} | | | | X | | |
| Concomitant medications, | Continuous monitoring | | | | X | |
| nonpharmacologic therapies, | | | | | | |
| and procedures | | | | | | |
| Adverse events | Continuous monitoring | | | | X | |
| Clinical chemistry | | X | X | X | X^{b} | |
| Hematology and coagulation | | X | X | X | | |
| Urinalysis and sediment | | X | X | X | | Obtain sample from a morning void |
| | | | | | | prior to dosing |
| Particpant safety card | | X | X | X | X | Confirm participants carry safety |
| review | | | | | | card at all times |

Pharmacokinetic and Pharmacodynamic Assessments

| Blood samples for PK/PD | | T/P | T/P | X | | Collect samples at any time during |
|---|---|---|---|---|---|---|
| | | | | | | the week 50/ED visit |
| Blood samples for ADA | | T | T | X | | Collect samples at any time during |
| | | | | | | the week 50/ED visit |
| | Extension Period | | | | | |
| | Visit | | | | | |
| | 12 | 13 | 14 | 15 | | |
| | Week | | | | | Notes |
| | W26 | W34 | W42 | W50/ED^{a} | Eval. for Renal | Additional visits can be |
| | Days and Window | | | | Flare and | performed as needed. An ED Visit |
| | | D239 ± | D295 ± | D351 ± | Extrarenal | should be performed if |
| Period | D183 | 7 | 7 | 7 | SLE Flare | participants discontinue early. |

Exploratory Assessments

| | | | | | |
|---|---|---|---|---|---|
| Sf-36 | | | X | | |
| EQ-5D-5L | | | X | | |
| FACIT-Fatigue | | | X | | |
| SLEDAI-2K | | | X | X | Perform as needed for evaluation of |
| | | | | | Extrarenal SLE Flare |
| Blood samples for anti- | | | X | X | |
| dsDNA and anti-C1q | | | | | |
| Blood and urine sample for | X | X | X | X | |
| biomarkers | | | | | |
| Option kidney biopsy^{f} | | | X^{g} | X^{f} | Send the local pathology report and |
| | | | | | microscopy slides to the Central |
| | | | | | Pathology Laboratory |

Administration of Study Intervention

| Background LN therapy | Continuous monitoring | | | X | |
|---|---|---|---|---|---|
| Ravulizumab or placebo | X | X | X | | Administer after all other required |
| | | | | | tests/procedures |

Note:
During the Extension Period, participants in the LN cohort continue to receive their randomized allocation of study drug (ravulizumab or placebo).
^{a}For participants who discontinue the study prior to the end of the Extension Period, the ED visit is completed as soon as possible. In addition, a Follow-up Phone Call is performed 8 weeks following the participant's last dose of study drug to collect concomitant medications, nonpharmacological therapies and procedures, and AEs.
^{b}Weight are obtained at every visit and measured predose on dosing visits. The dose regimen is based on the last recorded study visit body weight. If the study drug is prepared the night before a visit, the weight from the most recent study visit is used.
^{c}Renal Flare and/or Severe Extrarenal SLE Flare may occur at any time through Week 50. Evaluation of Renal Flare requires a UPCR from a spot urine sample that is confirmed on a 24-hr urine collection as well as 2 serum creatinine samples obtained with a 2-week period. Evaluation of Renal and Extrarenal SLE Flare is performed as soon as possible upon notification to the Investigator of symptom onset. If Renal Flare or Extrarenal SLE Flare occurs between scheduled visits, only the assessments for the Renal Flare/Extrarenal SLE Flare visit are needed. If Renal Flare or Extrarenal SLE Flare occur on a scheduled visit, all scheduled assessments are performed for that visit as well as any additional assessments required for the evaluation of the flare.
^{d}Vital sign measurements include systolic and diastolic BP, pulse oximetry, heart rate, respiratory rate, and temperature. On dosing days, vital signs are taken predose.
^{e}Single 12-lead ECG is collected at any time on Day 351 and the ED visit. Participants are supine for approximately 5 to 10 minutes before ECG collection and remain supine but awake during ECG collection.
^{f}Participants may have a kidney biopsy for clinical reasons or for evaluation of a Renal Flare at the discretion of the Investigator. The local pathology report and microscopy slides from kidney biopsies performed at other times during the study prior to Week 86 are sent to the Central Pathology Laboratory as soon as possible. Because examination of the kidney biopsy pathology may potentially unblind the study treatment (ravulizumab or placebo), Investigators and study site personnel do not examine the biopsy pathology for immunohistochemistry of complement prior to Week 50.
^{g}Participants are asked to undergo an optional repeat kidney biopsy after completion of the Extension Period. If a participant agrees to a repeat renal biopsy, it is performed at the Week 50 Visit or within 4 weeks (by Week 54).
Abbreviations: ADA = antidrug antibody;
AE = adverse event;
B = baseline; BP = blood pressure;
C3, C4, C1q = complement components 3, 4, and C1q;
CH50 = 50% hemolytic complement activity;
D = day;
dsDNA = double-stranded DNA;
ED = early discontinuation;
EQ-5D-5L = European Quality of Life Health 5-item questionnaire dimensions 5 level;
eGFR = estimated glomerular filtration rate;
Eval. = evaluation;
FACIT = Functional Assessment of Chronic Illness Therapy;
IgAN = immunoglobulin A nephropathy;
LN = lupus nephritis;
P = postdose;
PD = pharmacodynamics;
PE = physical examination;
PK = pharmacokinetics;
SLE = systemic lupus erythematosus;;
SLEDAI-2K = Systemic Lupus Erythematosus Disease Activity Index Safety of Estrogen in Lupus Erythematosus National Assessment (SELENA) Modification;
SF-36 = Short Form (36) Health Survey;
T = trough (predose);
UPCR = urine protein to creatinine ratio;
W = week;
WOCBP = women of childbearing potential.

TABLE 8
Schedule of Activities During the Extension Period: Week 26
Dose Administration, Week 28 to Week 50 Visits (IgAN Cohort)

| | Extension Period | | | | | |
|---|---|---|---|---|---|---|
| | Visit | | | | | |
| | 11 | 12 | 13 | 14 | 15 | |
| | Week | | | | | |
| | W26 | W28 | W36 | W44 | W50/ED^{a} | Notes |
| | Days and Window | | | | | Additional visits are performed as needed. |
| | | D197 ± | D253 ± | D309 ± | D351 ± | An ED Visit is performed if participants |
| Period | D183 | 7 | 7 | 7 | 7 | discontinue early. |

General Assessments/Procedures

| | | | | | |
|---|---|---|---|---|---|
| Weight^{b} | X | X | X | X | |
| Pregnancy test | X | X | X | X | Serum pregnancy test required at ED; urine |
| (WOCBP only) | | | | | pregnancy test all other visits. |

Efficacy Assessments

| | | | | | |
|---|---|---|---|---|---|
| 24-hr urine Collection | | | | X | Obtain 2 valid 24-yr urinie collections within |
| | | | | | 2 weeks of the week 50 Visit. |
| Morning spot urine sample | X | X | X | X | Obtain sample predose on dosing visits |
| eGRF | X | X | X | X | |
| Blood sample for C3, C4, | X | X | X | X | |
| and CH50 | | | | | |

Safety Assessments

| Physical examiniation | | | | | X | |
|---|---|---|---|---|---|---|
| Abbreviated physical | | X | X | X | | |
| examination | | | | | | |
| Vital signs^{c} | | X | X | X | X | |
| ECG^{d} | | | | | X | |
| Concomitant medications, | Continuous monitoring | | | | | |
| nonpharmacologic therapies, | | | | | | |
| and procedures | | | | | | |
| Adverse events | Continuous monitoring | | | | | |
| Clinical chemistry | | X | X | X | X | |
| Hematology and coagulation | | X | X | X | X | |
| Urinalysis and sediment | | X | X | X | X | Obtain sample from a morning void prior to |
| | | | | | | dosoing |
| Participant safety acard review | | X | X | X | X | Confirm participants carry safety card at all |
| | | | | | | times |

Pharmacokinetic and Pharmacodynamic Assessments

| | | | | | |
|---|---|---|---|---|---|
| Blood samples for PK/PD | T/P | T/P | T/P | X | Collect samples at any time during the Week |
| | | | | | 50/ED visit |
| Blood samples for ADA | T | T | T | X | Collect samples at any time during the Week |
| | | | | | 50/ED visit |

Exploratory Assessments

| SF-36 | | | | | X | |
|---|---|---|---|---|---|---|
| EQ-5D-5L | | | | | X | |
| Blood and urine samples for | | X | X | X | X | |
| biomarkers | | | | | | |
| Kidney biopsy (if indicated | Continuous monitoring | | | | | Send the site pathology report and |
| per Investigator)^{e} | | | | | | microscopy slides to the Central Pathology |
| | | | | | | Laboratory |
| | Extension Period | | | | | |
| | Visit | | | | | |
| | 11 | 12 | 13 | 14 | 15 | |
| | Week | | | | | Notes |
| | W26 | W28 | W36 | W44 | W50/ED^{a} | Additional visits can be |
| | Days and Window | | | | | performed as needed. An ED Visit |
| | | D197 ± | D253 ± | D309 ± | D351 ± | should be performed if |
| Period | D183 | 7 | 7 | 7 | 7 | participants discontinue early. |

Administration of Study Intervention

| Background IgAN therapy | Continuous monitoring | | | | |
|---|---|---|---|---|---|
| Ravulizumab^{f} | X | X | X | X | Administer after all other required |
| | | | | | tests/procedures |

Note:
During the Extension Period, the IgAN placebo group switch to ravulizumab, such that all IgAN participants treated with ravulizumab when the study becomes open label for the IgAN group. Participants in the ravulizumab group receive a blinded ravulizumab dose of 900 mg at Week 26 and continue to receive the weight-based dosing thereafter.
^{a}For participants who discontinue the study prior to the end of the Extension Period, the ED visit is completed as soon as possible. In addition, a Follow-up Phone Call is performed 8 weeks following the participant's last dose of study drug to collect concomitant medications, nonpharmacological therapies and procedures, and AEs.
^{b}Weight is obtained at every visit and measured predose on dosing visits. The dose regimen is based on the last recorded study visit body weight. If the study drug is prepared the night before a visit, the weight from the most recent study visit is used.
^{c}Vital sign measurements include systolic and diastolic BP, pulse oximetry, heart rate, respiratory rate, and temperature. On dosing days, vital signs are taken predose.
^{d}Single 12-lead ECG is collected at any time on Day 351 and the ED visit. Participants are supine for approximately 5 to 10 minutes before ECG collection and remain supine but awake during ECG collection.
^{c}Participants can receive kidney biopsy (performed at the discretion of the Investigator for clinical reasons as part of standard of care). The local pathology report and microscopy slides from kidney biopsies performed at other times during the study prior to Week 86 are sent to the Central Pathology Laboratory as soon as possible. Since examination of the biopsy pathology results can potentially unblind the study treatment (ravulizumab or placebo) Investigators and study site personnel do not examine the biopsy pathology results for immunohistochemistry of complement prior to Week 50.
^{f}Participants in the IgAN cohort receive weight based doses of ravulizumab q8w until the end of the Extension Period.
Abbreviations: ADA = antidrug antibody;
AE = adverse event;
B = baseline;
BP = blood pressure;
C3, C4, C1q = complement components 53, 4, and C1q;
CH50 = 50% hemolytic complement activity;
D = day;
ECG = electrocardiogram;
ED = early discontinuation;
eGFR = estimated glomerular filtration rate;
EQ-5D-5L = European Quality of Life Health 5-item questionnaire dimensions 5 level;
FACIT = Functional Assessment of Chronic Illness Therapy;
IgAN = immunoglobulin A nephropathy;
LN = lupus nephritis;
P = postdose;
PD = pharmacodynamics;
PE = physical examination;
PK = pharmacokinetics;
q8w = once every 8 weeks;
SF-36 = Short Form (36) Health Survey;
T = trough (predose);
UPCR = urine protein to creatinine ratio;
W = week;
WOCBP = women of childbearing potential.

TABLE 9
Schedule of Activities During Post-Treatment Follow-up Period (Both LN and IgAN Cohorts)

| Period | |
|---|---|
| Post-Treatment Follow-up Period | Notes |

| Visit | | | |
|---|---|---|---|
| 15 | 16 | 17 | 18 |
| Week | | | |
| W 52^{a} | | | |
| (IgAN | | | |
| only) | W 62 | W 74 | W 86/EoS |
| Days and Window | | | |
| D 354 ± 7 | D 435 | D 519 | D 603 |

Efficacy Assessments

| Record UPCR results (local | | X | X | X | Record local laboratory values from the |
|---|---|---|---|---|---|
| laboratory) | | | | | participant's most recent testing prior to or |
| | | | | | on the study visit. Spot urine samples are |
| | | | | | sufficient; morning voids are preferred. |
| Record serum creatinine results | | X | X | X | Record local laboratory values from the |
| (local laboratory) | | | | | participant's most recent testing prior to or |
| | | | | | on the study visit |
| Monitor for Renal Flare and | | X | X | X | Document in the participant's CRF for the |
| Extrarenal SLE Flare | | | | | 12-week period since the previous study |
| (LN cohort) | | | | | visit. |
| Monitor for renal disease | | X | X | X | |
| progression (IgAN cohort) | | | | | |
| Background therapy for LN or | Continuous monitoring | | | | |
| IgAN | | | | | |

Safety Assessments

| | | |
|---|---|---|
| Concomitant medications, | X | Phone visit |
| nonpharmacologic therapies, and | | |
| procedures | | |
| Adverse events | X | Phone visit |

^{a}For participants in the IgAN cohort, a Follow-up Phone Call is performed 8 weeks following the participant's last dose of study drug to collect information on concomitant medications, nonpharmacological therapies, and procedures, and AEs.
Abbreviations: D = day; EoS = End of Study; IgAN = immunoglobulin A nephropathy; LN = lupus nephritis; SLE = systemic lupus erythematosus; UPCR = urine protein to creatinine ratio; W = week.

3. Overview of Objectives of Endpoints

The primary objective of the study for both cohorts is to evaluate the efficacy of ravulizumab compared with placebo to reduce proteinuria in adult participants with LN or IgAN. This objective is assessed based on percentage change in proteinuria from baseline to Week 26 (based on 24-hr urine collection[s] at each time point).

A secondary objective for both cohorts is to evaluate the efficacy of ravulizumab compared with placebo to improve measures of kidney function in adult participants with LN or IgAN (e.g., via (1) percentage change in proteinuria from baseline to Week 50 (based on 24-hr urine collection[s] at each time point), (2) percentage of participants with >30% and >50% reduction in proteinuria at Week 26 and Week 50 compared to baseline (based on 24-hr urine collection[s] at each time point), (3) change from baseline in eGFR at Week 26 and Week 50, and (4) absolute values and change from baseline in serum C3 and C4 concentrations at Week 26 and Week 50).

A secondary objective for the LN cohort only is to evaluate the efficacy of ravulizumab compared with placebo to improve measures of kidney function in adult participants with LN (e.g., via (1) percentage of participants meeting the criteria for CRR at Week 26 and Week 50, (2) percentage of participants meeting the criteria for PRR at Week 26 and Week 50, (3) time to UPCR<0.5 g/g as measured by spot urine sample, (4) percentage of participants achieving corticosteroid taper to 7.5 mg/day at Weeks 14, 26, and 50, (5) Percentage of participants with Renal Flare through Week 50, (6) Percentage of participants with Extrarenal SLE Flare through Week 50, (7) percentage of participants with Treatment Failure through Week 50, and (8) absolute values and change from baseline in serum albumin at Week 26 and Week 50.

A secondary objective for the IgAN cohort only is to evaluate the efficacy of ravulizumab compared with placebo on measures of kidney function in adult participants with IgAN (e.g., via percentage of participants meeting the criteria for Partial Remission at Week 26 and Week 50).

PK/PD/Immunogenicity objectives for both cohorts include characterizing the PK/PD of ravulizumab in adult participants with LN or IgAN (e.g., via (1) absolute values and change from baseline in total C5 and free C5 concentrations over time and (2) absolute values and change from baseline in ravulizumab concentrations over time), as well as characterizing the potential for immunogenicity of ravulizumab in adult participants with LN or IgAN (e.g., incidence of ADAs over time).

A safety objectives for both cohorts is to characterize the safety and tolerability of ravulizumab in adult participants with LN or IgAN (e.g., via incidence of AEs and SAEs over time).

Exploratory objectives for both cohorts include (1) evaluating the efficacy of ravulizumab compared with placebo on hematuria in adult participants with LN or IgAN (e.g., effect on hematuria as measured by absolute value and change from baseline in RBC in urine from baseline to Week 26 and Week 50 and percentage of participants with ≥10 RBC), (2) assessing quality of life based on participant reported outcomes in adult participants with LN or IgAN based on treatment with ravulizumab compared with placebo (e.g., change from baseline in SF-36 at Week 26 and Week 50 and change from baseline in EQ-5D-5L at Week 26 and Week 50), (3) evaluating complement and autoimmune biomarkers in adult participants with LN or IgAN (e.g., absolute values and change from baseline in levels of biomarkers in blood, urine, and kidney tissue at Week 26 and Week 50).

Exploratory objectives for the LN cohort only include (1) assessing the efficacy of ravulizumab in exploratory efficacy endpoints (e.g., time to CRR and PRR (using spot UPCR), percentage of participants with Overall Renal Response at Week 26 and Week 50 (CRR and PRR), and time to UPCR>50% decrease from baseline (using spot UPCR)), (2) assessing quality of life based on participant reported outcomes (change from baseline in FACIT Fatigue score at Week 26 and Week 50), and (3) assessing the efficacy of ravulizumab in other exploratory endpoints (absolute values and change from baseline in anti-dsDNA and anti-C1q antibodies at Week 26 and Week 50 and histology changes from baseline to Week 50).

Exploratory objectives for the IgAN cohort only include assessing the efficacy of ravulizumab in exploratory efficacy endpoints (slope of eGFR computed from baseline to Week 26 and Week 50).

4. Definitions for Endpoints

a. Renal (LN Cohort Only)

Renal Flare is determined in the opinion of the Investigator in addition to the criteria outlined below. For participants who achieve CRR, a Renal Flare is the reproducible recurrence of proteinuria>1 g/g.

For all other participants, a Renal Flare is either of the following: Reproducible increase of serum creatinine>25% higher than baseline or above the upper limit of normal, plus any one of the following: Reproducible proteinuria≥75% higher than baseline, worsening active urinary sediment compared to baseline as defined by an increase of ≥5 RBCs/high power field (hpf) or new RBC casts (based on local laboratory results from at least 2 samples), or Kidney biopsy newly conducted since the biopsy used for eligibility demonstrating LN Class III or IV activity.

Reproducible doubling of the UPCR from a 24 hour urine collection compared with the lowest previous value obtained after the first dose of study drug.

Reproducibility of proteinuria requires that the proteinuria based on a UPCR from a morning spot urine collection is confirmed by UPCR calculated on a 24 hour urine collection obtained within a 2 week period.

Reproducibility of serum creatinine requires 2 blood tests within 2 week period.

Participants who meet criteria for the protocol-defined Renal Flare will receive Rescue Therapy (as defined in Section 6.6) The Medical Monitor should be notified of the Renal Flare by the Investigator or Sub-investigator. Any renal flare that does not meet the protocol-defined Renal Flare criteria may be treated with a limited duration of increased oral corticosteroids (<14 days) after discussion with the Medical Monitor. Such treatment will not be considered Rescue Therapy and will not be considered Treatment Failure. Renal Flare criteria will be recorded on the Renal Flare case report form (CRF).

b. Extrarenal Systemic Lupus Erythematosus Flare (LN Cohort Only)

Extrarenal SLE Flare is defined as an increase in Systemic Lupus Erythematosus Disease Activity Index Safety of Estrogen in Lupus Erythematosus National Assessment (SELENA) Modification (SLEDAI-2K)>4 points that is not accounted for by proteinuria, hematuria, urinary cellular casts, hypocomplementemia, or an increase in anti double-stranded DNA (anti-dsDNA) antibody level. Participants in the LN cohort who meet the criteria for Extrarenal SLE Flare can receive Rescue Treatment, if considered clinically appropriate by the Investigator. If Rescue Therapy is administered, the event is considered a Severe Extrarenal SLE Flare.

Participants are allowed to receive a limited duration of increased corticosteroids (<14 days) for non-severe extrarenal SLE flare, if clinically warranted. Such treatment is not considered Rescue Therapy and is not considered Treatment Failure.

c. Treatment Failure (LN Cohort Only)

Treatment Failure is defined as the occurrence of any of the following events: receipt of Rescue Therapy at any time up to Week 50 for protocol-defined Renal Flare or Severe Extrarenal SLE Flare.

Increase in corticosteroids for extrarenal SLE flare not meeting the protocol definition of Severe Extrarenal SLE Flare, renal flare not meeting protocol definition for Renal Flare, other medical conditions or surgery are not included in Treatment Failure. A limited duration of increased corticosteroids (<14 days) is not considered as Rescue Therapy.

Participants who meet the criteria for Treatment Failure can continue to receive the study drug.

d. Complete and Partial Renal Response (LN Cohort Only)

Complete Renal Response and Partial Renal Response (PRR) will be assessed at Week 26 and Week 50. To achieve CRR, participants in the LN cohort must meet all 3 of the following criteria:
(1) A decrease in mean UPCR to ≤0.5 g/g based on two 24-hr urine collections obtained within 2 weeks prior to the study visit (Week 26 or Week 50)
(2) Estimated glomerular filtration rate (eGFR)>60 mL/min/1.73 m2 or no eGFR reduction≥20% from the baseline value based on the mean of 2 values. The first eGFR value must be obtained within 2 weeks prior to the study visit (Week 26 or Week 50) and the second eGFR value will be obtained on the study visit (Week 26 or Week 50).
(3) No Treatment Failure.

To achieve PRR, participants in the LN cohort must meet all 3 of the following criteria:
(1) A decrease in UPCR>50% compared to the baseline value based on mean of two 24 hr urine collections obtained within 2 weeks prior to the study visit (Week 26 or Week 50);
(2) Estimated glomerular filtration rate (eGFR)≥60 mL/min/1.73 m2 or no eGFR reduction≥20% from the baseline value based on the mean of 2 values. The first eGFR value must be obtained within 2 weeks prior to the study visit (Week 26 or Week 50) and the second eGFR value will be obtained on the study visit (Week 26 or Week 50.
(3) No Treatment Failure.

e. Partial Remission (IgAN Cohort Only)

Partial Remission is defined as mean proteinuria<1 g/24-hrs based on 2 valid 24-hr urine collections obtained within 2 weeks prior to the study visit (Week 26 or Week 50).

5. Inclusion and Exclusion Criteria

To be eligible to participate in the study, participants in both cohorts must meet all of the below criteria.
1. Participant must be ≥18 and ≤75 years of age at the time of signing the informed consent;
2. Body weight≥40 kg at Screening;
3. Male or female. Female participants of childbearing potential, male participants, and male participants with female partners of childbearing potential must follow protocol specified contraception guidance;
4. Capable of giving informed consent;
5. To reduce the risk of meningococcal infection (N meningitidis), all participants must be vaccinated against meningococcal infection from serogroups A, C, W, Y, and B within 3 years prior to, or at the time of, randomization according to national/local guidelines. Participants who do not meet this requirement are vaccinated against meningococcal infection prior to randomization according to national/local guidelines and receive prophylactic antibiotics for at least 2 weeks after meningococcal vaccination if randomization occurs <2 weeks after initial vaccination;
6. All participants must also receive vaccinations for Haemophilus influenzae type b (Hib) and Streptococcus pneumoniae prior to randomization, unless previously vaccinated, according to current national/local vaccination guidelines;
7. Local pathology report from the biopsy used for diagnosis must be available; and
8. Participants on SGLT-2 inhibitors (e.g., empagliflozin) must be on a stable dose for ≥3 months with no planned change in dose during the study.

In addition, to be included in the LN Cohort, the participant must meet the following criteria:
1. Clinical diagnosis of SLE by 2019 The American College of Rheumatology (ACR) and European League Against Rheumatism (EULAR) criteria;
2. Diagnosis of 2018 Revised ISN/RPS classification (active focal or diffuse proliferative LN Class III or IV confirmed by biopsy obtained ≤6 months prior to Screening or during Screening Period. Participants may co-exhibit Class V disease. Participants with de novo or relapsing disease may be eligible;
3. Clinically active LN at Screening requiring/receiving immunosuppression induction treatment in the opinion of the Investigator; and
4. Proteinuria with UPCR≥1 g/g based on one 24-hour urine collection during the Screening Period.

In addition, to be included in the IgAN Cohort, the participant must meet the following criteria:
1. Established diagnosis of primary IgAN based on kidney biopsy obtained any time prior to or during the Screening Period;
2. Mean proteinuria≥1 g/day on 2 complete and valid 24-hour urine collections during the Screening Period;
3. Presence of hematuria as defined by 1+blood based on urine dipstick or ≥10 red blood cell (RBC)/hpf microscopy on urine sediment (performed by the local laboratory);
4. Compliance with stable and optimal dose of RAS inhibitor treatment including maximum allowed or tolerated ACE inhibitor and/or angiotensin receptor blocker dose for ≥3 months prior to Screening with no expected change in dose during the study; and
5. Controlled and stable blood pressure over the past 3 months <140/90 mmHg.

Participants from both cohorts are excluded from the study if any of the below criteria apply.
1. Estimated GFR<30 mL/min/1.73 m2 during Screening calculated by CKD-EPI;
2. More than or equal to 50% interstitial fibrosis, tubular atrophy, glomerular sclerosis, or crescent formation in glomeruli on most recent kidney biopsy prior or during the Screening Period;
3. Concomitant significant renal disease other than LN or IgAN on the most recent biopsy prior to or during the Screening Period; 4. History of kidney transplant or planned kidney transplant during the Treatment
Period;
5. History of other solid organ (heart, lung, small bowel, pancreas, or liver) or bone marrow transplant; or planned transplant during the Treatment Period;
6. Splenectomy or functional asplenia;
7. Known medical or psychological condition(s) or risk factor that, in the opinion of the Investigator, might interfere with the participant's full participation in the study, pose any additional risk for the participant, or confound the assessment of the participant or outcome of the study;
8. Known or suspected history of drug or alcohol abuse or dependence within 1 year prior to the start of the Screening Period;
9. History of malignancy within 5 years of Screening with the exception of nonmelanoma skin cancer or carcinoma in situ of the cervix that has been treated with no evidence of recurrence;
10. Known history of hepatitis B or C viral infection;
11. Known history of HIV infection (evidenced by HIV type 1 or type 2 [HIV 1, HIV 2] antibody);
12. Bone marrow insufficiency with absolute neutrophil count<1.3×103/μL; thrombocytopenia (platelet count<50,000/mm3);
13. Active systemic bacterial, viral, or fungal infection within 14 days prior to randomization;
14. History of N meningitidis infection;
15. Inability to take or tolerate the standard of care background therapies;
16. Received biologic, including but not limited to belimumab or rituximab, ≤6 months prior to Screening;
17. Previously received a complement inhibitor (e.g., eculizumab) at any time;
18. Participation in another investigational drug or investigational device study within 30 days before initiation of study drug on Day 1 in this study or within 5 half-lives of that investigational product, whichever is greater; or
19. Pregnant, breastfeeding, or intending to conceive during the course of the study.

In addition, participants from the LN cohort are excluded from the study if any of the below criteria apply.
1. Participants who have received any of the following treatments after their qualifying kidney biopsy used for eligibility: cyclophosphamide≤6 months of Screening, calcineurin inhibitors≤3 months of Screening, a cumulative dose of IV methylprednisolone>3 g, mycophenolate mofetil>2 g/day (or equivalent) for ≥4 consecutive weeks, or oral corticosteroids≥0.5 mg/kg/day for ≥4 consecutive weeks;
2. Uncontrolled hypertension (systolic blood pressure>160 or diastolic blood pressure>110 mmHg) on 2 or more measurements during the Screening Period; or
3. Clinically active SLE-related cerebritis, seizures, pericarditis, stroke, or stroke syndrome requiring treatment.

In addition, participants from the IgAN cohort are excluded from the study if any of the below criteria apply.
1. Diagnosis of rapid progressive glomerulonephritis as measured by eGFR loss≥30% over a period of 3 months prior to or during the Screening Period;
2. Secondary etiologies of IgAN (eg, SLE, cirrhosis, celiac disease);
3. Clinically active Henoch-Schonlein purpura (IgA vasculitis) requiring treatment
4. Prednisone or prednisone equivalent>20 mg for >14 consecutive days or any other immunosuppression within 6 months of Screening;
5. Blood pressure of ≥140/90 mmHg during the Screening Period confirmed on 2 measures>30 minutes apart; or
6. Body mass index≥35.

6. Study Drug

Ravulizumab is formulated at pH 7.0 and is supplied in 30 mL single-use vials. Each vial of ravulizumab contains 300 mg of ravulizumab (10 mg/mL) in 10 mM sodium phosphate, 150 mM sodium chloride, 0.02% polysorbate 80, and water for injection. The comparator product (placebo) is formulated as a matching sterile, clear, colorless solution with the same buffer components, but without active ingredient. Additional details are presented in Table 1.

The dosing regimen (Table 2) consists of a loading dose followed by maintenance dosing administered q8w. The maintenance dosing is initiated 2 weeks after the loading dose administration. Weight-based dosing is based on the participant's body weight recorded at the day of the infusion visit. If the weight at the day of the infusion cannot be obtained, the weight recorded during the most recent prior study visit can be used.

At the scheduled dosing visits, study drug is administered after all other tests and procedures have been completed, excluding the postdose sample collections (PK/PD/biomarkers).

During the Initial Evaluation Period (Day 1 through Week 26), participants in each cohort are randomized 2:1 to receive blinded doses of ravulizumab or placebo.
Ravulizumab group: participants receive a blinded loading dose of ravulizumab via IV infusion on Day 1, followed by a blinded maintenance doses at Week 2 then q8w thereafter through the end of the Initial Evaluation Period
Participants in the placebo group receive a blinded matching placebo dose via IV infusion on Day 1, followed by a blinded matching placebo dose at Week 2, then q8w thereafter through the end of the Initial Evaluation Period.

During the Extension Period (Week 26 through Week 50), participants in the LN cohort continue on the same maintenance regimen. In the IgAN cohort, participants in the placebo group switch to receive a blinded loading dose of ravulizumab at Week 26 and participants in the ravulizumab group receive a blinded ravulizumab dose of 900 mg at Week 26. Starting at Week 28, all participants in the IgAN cohort receive open-label weight-based doses of ravulizumab (Table 3) q8w until the end of the Extension Period.

7. Background Therapy for LN Cohort

During the course of the study, participants in the LN cohort receive background therapy consistent with the standard of care for induction and maintenance treatment of LN.

For participants who have not started corticosteroid induction treatment prior to Screening:
1. Participants receive a cumulative dose of 1 gram of methylprednisolone IV administered in 1 or multiple divided doses during the Screening Period (prior to Day 1).
2. During the Screening Period and no later than Day 2, all participants receive oral corticosteroids with prednisone or prednisone equivalent 0.5 mg/kg/day. The starting minimum and maximum dose allowed are 30 mg/day and 60 mg/day, respectively. A corticosteroid taper commences on Week 2 (Day 14) as outlined in Table 4.
3. During the Screening Period and no later than Day 1, participants receive a cumulative dose of 1 to 1.5 g/day of MMF any time after completion of the IV methylprednisolone during the Screening Period and no later than Day 1. The dose can be administered in multiple divided doses. Participants continue to receive 1 to 1.5 g/day for 1 week.
4. After receiving 1 to 1.5 g/day for 1 week, the dose is increased per the discretion of the Investigator to a cumulative dose of 2 to 2.5 g/day of MMF no later than by Week 4 (Day 28). The dose can be administered in multiple divided doses. Participants continue to receive 2 to 2.5 g/day of MMF for a minimum duration of 50 weeks after which it may be decreased or discontinued based on the Investigators' judgment and the KDIGO clinical practice guidelines (KDIGO Clinical practice guideline for glomerulonephritis. Kidney International Supplements. 2012; 2(2):140).

For participants who have initiated corticosteroid induction treatment prior to Screening and do not meet Exclusion Criterion:
1. If the participant already received methylprednisolone IV≥1 g or equivalent and is receiving MMF≥2 g/day prior to Screening, then methylprednisolone IV is not given and MMF w is continued at the current dose for a minimum duration of 50 weeks, after which it may be decreased or discontinued based on the Investigator's judgment and the KDIGO clinical practice guidelines.
2. If the participant already received methylprednisolone IV≥1 g or equivalent and is receiving MMF<2 g/day, then methylprednisolone IV is not given and the MMF dose is increased during the Screening Period (no later than Day 1) to a cumulative dose of 1 to 1.5 g/day. Participants continue to receive 1 to 1.5 g/day for 1 week after which the MMF dose is increased per the discretion of the Investigator to 2 to 2.5 g/day to be achieved no later than Week 4 (Day 28). These doses can be administered in multiple divided doses. Participants continue to receive 2 to 2.5 g/day for a minimum duration of 50 weeks, after which it may be decreased or discontinued based on the Investigator's judgment and the KDIGO clinical practice guidelines.
3. If a participant is already receiving prednisone or prednisone equivalent, the dose is continued until Day 2 at which time 0.5 mg/kg/day should be administered (the minimum and maximum dose allowed are 30 mg/day and 60 mg/day, respectively). The prednisone dose is tapered starting on Week 2 (Day 14) according to the schedule in Table 4.

Other considerations regarding MMF dosing:
1. An equivalent dose of enteric-coated mycophenolic acid sodium (MPS) can be used instead of MMF (i.e., 360 mg dose MPS is equivalent to a 500 mg dose of MMF)
2. Investigators can adjust the dosage of MMF due to tolerance or AEs. After the symptoms resolve, the Investigator can attempt to increase MMF (or equivalent) to the goal level. If symptoms return, then the participant is continued on the highest tolerable dose.
3. Any changes to the dose of MMF and the justification are documented in the CRF.

Other considerations regarding the corticosteroid taper:
5. All participants have a scheduled corticosteroid taper starting on Day 14. Participants reduce their prednisone dose according to their baseline body weight over 10 weeks until the dose is 7.5 mg/day by Week 12 (Table 4).
6. Deviations from the scheduled corticosteroid taper for any reason other than Renal Flare or Extrarenal SLE Flare confound interpretation, so every attempt should be made to adhere to the tapering schedule.
7. If disease is too clinically active in the opinion of the Investigator to begin the corticosteroid taper after Week 2, then the participant can continue to receive his or her initial corticosteroid dose for up to an additional 28 days. Similarly, participants who have started the taper and whose disease is too clinically active to continue tapering, can remain at the same taper dose achieved for up to an additional 28 days. Failure to achieve the corticosteroid taper by Week 12 is not be considered as Treatment Failure and is captured as a secondary endpoint.
8. However, the prednisone dose can NOT be increased beyond the taper dose achieved unless participant meets the protocol-defined criteria for Renal Flare and/or Severe Extrarenal SLE Flare in which case these participants receive Rescue Therapy and are included as Treatment Failures.

8. Background Therapy for IgAN Cohort

The background therapies for participants in the IgAN cohort is consistent with standard of care and include the maximumly tolerated dose of RAS-blocking agents, such as ACE inhibitors or ARBs. The background treatment is held stable throughout the Treatment Period of the study.

9. Rescue Therapy for LN Cohort

Participants in the LN cohort receive Rescue Therapy in the event of a protocol-defined Renal Flare or Severe Extrarenal SLE Flare. Rescue Therapy is defined as intensification of current standard of care or introduction of new immunosuppressive therapies.

The specific choice of Rescue Therapy(ies) is generally at the discretion of the Investigator. However, the following guidelines for corticosteroid dosing for protocol-defined Renal Flare and Severe Extrarenal SLE Flares should be considered to maintain treatment consistency:
1. Participants with protocol-defined Renal Flare can be treated with prednisone up to 0.5 mg/kg/day (not to exceed 60 mg/day) for up to 2 weeks. Prednisone can then be tapered weekly to 10 mg/day within 6 weeks after the initial prednisone increase. Prednisone can further be tapered to 7.5 mg/day at the discretion of the Investigator.
2. Participants with Severe Extrarenal SLE flare can be treated with prednisone up to 1 mg/kg/day (not to exceed 60 mg/day) for up to 2 weeks. Prednisone is then be tapered every 2 weeks to achieve 7.5 mg/day within 12 weeks after the initial corticosteroid increase.
3. Intravenous corticosteroids in equivalent doses can be allowed if gastrointestinal involvement temporality precludes oral corticosteroid use.

Prednisone≥10 mg for ≤14 days is not considered Rescue Therapy in the following instances: (1) renal flares not meeting the protocol defined criteria for Renal Flare, (2) extrarenal SLE flares not requiring >14 days of >10 mg prednisone or equivalent or introduction of new immunosuppressive medication in the opinion of Investigator; and (3) other medical conditions or surgery.

10. Concomitant Therapy

Any medication or therapy (including over-the-counter or prescription medicines, vaccines, vitamins, and/or herbal supplements) deemed necessary for the participant's care during the study, or for the treatment of any AE, along with any other medications, other than those listed as disallowed medications, can be given at the discretion of the Investigator.

If adequate blood pressure control is not achieved during the study, participants can receive additional antihypertensive agents, but not agents that affect proteinuria during the study. It is recommended that NSAIDs not be initiated during the study due to the possibility of adverse effects on renal function. They may be used, however, if necessary for the control of symptoms.

For participants in the LN cohort: (1) pneumocystis pneumonia prophylaxis is allowed at the discretion of the Investigator, (2) treatment with antimalarial agents such as hydroxychloroquine are allowed unless contraindicated, and (3) measures to prevent and treat osteoporosis are strongly encouraged during the study; these measures may include any, or all, of the following: calcium carbonate or citrate, Vitamin D, and bisphosphonates.

Participants in both cohorts are prohibited from receiving any of the following medications and therapies during the entire duration of study participation: (1) experimental interventions or therapies, (2) eculizumab, and (3) SGLT-2 inhibitors and direct renin antagonists.

In the event that a participant receives a prohibited medication and/or therapy, the participant should discontinue study drug with the exception of SGLT-2 inhibitors and direct renin antagonists (SGLT-2 inhibitors and direct renin antagonists are prohibited but may not require discontinuation of study drug based on the discussion and approval of the Investigator and Medical Monitor).

Participants in the IgAN cohort are also prohibited from receiving any of the following medications and therapies during the entire duration of study participation: (1) hydroxychloroquine, (2) immunosuppressive agents (e.g., MMF), and (3) systemic corticosteroids for >14 consecutive days (short-term steroid course for ≤14 days for medical conditions not related to IgAN or surgery are permitted).

11. General Assessments and Procedures

The diagnosis of LN and IgAN is based on a kidney biopsy obtained prior to or during the Screening Period. Eligibility is determined using the local pathology report according to standardized globally recognized guidelines, as follows. For participants in the LN cohort, kidney biopsies must have been obtained ≤6 months prior to Screening or during Screening Period; eligibility is based the ISN/RPS classification guidelines. For participants in the IgAN cohort, kidney biopsies may have been obtained any time prior to Day 1 (see e.g., Haas M., Am J Kidney Dis. 1997; 29(6):829-842, and Trimarchi H, et al., Kidney Int. 2017; 91(5):1014-1021).

The local pathology report is entered in the CRF during Screening according to the CRF completion guidelines. In particular, the degree of IgG, IgA, immunoglobulin M (IgM), C3, and C1q (both cohorts); the activity score/class (LN cohort only); and the MEST-C score (IgAN cohort only), are obtained from the local pathology reports, if available, and documented in the CRF.

Kidney biopsies can be performed any time during the study at the discretion of the Investigator for renal flare or other indications.

For participants in the LN cohort, a repeat biopsy at the end of the Extension Period (Week 50) is optional and can be performed up to Week 54.

A Central Pathology Laboratory is used to confirm the diagnosis on the kidney biopsy used for eligibility to minimize interpersonal variation in histological scoring. The Central Pathology Laboratory is blinded to treatment allocation. The Central Pathology Laboratory reviews: (a) all kidney biopsies used for eligibility for participants in the LN cohort, (2) kidney biopsies performed within 1 year of Screening or during Screening for participants in the IgAN cohort, and (3) all kidney biopsies performed during the study any time prior to ED or completion of the Extension Period (Week 50).

Due to its mechanism of action, the use of ravulizumab increases a participant's susceptibility to meningococcal infection due to N meningitidis. To reduce the risk of infection, all participants must be vaccinated within 3 years prior to or at the time of the first infusion of study drug. Participants who have not been vaccinated prior to starting study drug for any reason, should receive appropriate prophylactic antibiotics prior to and for at least 2 weeks after vaccination. Vaccines against serotypes A, C, Y, W135, and B where available, are recommended in preventing the commonly pathogenic meningococcal serotypes. Participants must receive the complete primary vaccination series and be revaccinated if indicated according to current national vaccination guidelines. Vaccination may not be sufficient to prevent meningococcal infection.

Participants are administered prophylactic antibiotics for meningococcal infection until at least 2 weeks after vaccination if randomization occurs <2 weeks after initial vaccination. Consideration is given per official guidance and local practice on the appropriate use of prophylactic antibacterial agents. All participants are monitored for early signs of meningococcal infection, evaluated immediately if infection is suspected, and treated with appropriate antibiotics, if necessary.

Meningococcal serogroups ACWY and B vaccinations are required during screening for participants who do not meet criteria for previous vaccination. The vaccination series is completed during the study according to national and local vaccination schedule guidelines. In participants with IgAN, every effort should be made to start the meningococcal vaccination series at least 14 days prior to randomization.

All participants must also be vaccinated against Hib and S pneumoniae prior to randomization, unless previously vaccinated, according to current national/local vaccination guidelines.

12. Efficacy Assessments

For the determination of proteinuria, 24-hour urine collection is obtained during Screening, Week 26, and Week 50 and is analyzed by a central laboratory. In addition to protein, albumin, sodium, and creatinine are also quantified in each of the 24-hour urine collection. Both protein to creatinine (UPCR) as well as albumin to creatinine ratios (UACR) are also calculated in an aliquot of the 24-hour urine collection.

Rigorous exercise and significant change in diet (in particular, salt intake) is avoided within 48 hours before collection of 24-hour urine samples, whenever possible.

The collection is obtained prior to or >7 days after administration of vaccine(s) or biopsy procedures and prior to administration of ravulizumab or placebo on dosing days.

For participants in the LN cohort, proteinuria is measured by UPCR. A single 24-hour urine collection is obtained at Screening to assess eligibility. Two separate 24-hour urine collections are obtained within 2 weeks prior to the Week 26 Visit (to assess the primary endpoint) and Week 50 visit (to assess a secondary endpoint). Confirmation of a protocol-defined Renal Flare requires a single 24-hour urine collection within 2 weeks of the spot urine sample.

Participants in the IgAN cohort are required to provide 2 separate complete and valid 24-hour urine collections during the Screening Period (to assess eligibility), at Week 26 (to assess the primary endpoint), and at Week 50 (to assess a secondary endpoint). The 2 valid 24-hour urine collections are obtained within 2 weeks before the Week 26 and Week 50 Visits.

Completeness of the 24-hour urine collection is estimated from rate of creatinine excretion. Normal values of creatinine excretion vary with age and body weight. Hence, a 24-hour urine collection is considered valid if all the following criteria are met, otherwise the urine collection is required to be repeated: (1) The collection is between 22 to 26 hours in duration (i.e., time from the initial discarded void to the last void/attempt to void), (2) no voids are missed between the start and end time of the collection as indicated by the participant's urine collection diary; (3) the 24-hour creatinine content is within 25% of expected range as estimated by the following formula: [(140−age)×weight]/5000, where weight is in kilograms. This result is multiplied by 0.85 in women (see, Ix JI-1, et al., Clin J Am Soc Nephrol. 2011; 6(1):184 191); and (4) the maximum variation in total 24-hour urine creatinine between the 2 urine collections must be ≤25%. If any of the collections do not meet the validity criteria outlined above, the collection must be repeated as soon as possible within the time frames outlined in the Schedule of Assessment in order to ensure that 2 valid collections are obtained for each of the study time points.

Urinary protein, albumin, and creatinine levels from morning spot urine sample prior to dosing are also measured during Screening and during the study per the Schedule of Assessments to assess the effect of ravulizumab on UPCR and urine albumin creatinine ratio (UACR). Two consecutive spot urine samples are obtained for participants in both disease cohorts at the Week 18 Visits. The spot urine sample is obtained prior to or >7 days after administration of vaccine(s) or biopsy procedures and prior to administration of ravulizumab or placebo on dosing days. Spot urine samples conducted as routine standard of care are used for UPCR during the Post-treatment Follow-up Period per the Schedule of Assessments.

Changes in renal function are monitored using measurements of Estimated Glomerular Filtration Rate (eGFR) (mL/min/1.73m2) and creatinine clearance on a 24-hour urine collection as outlined in the Schedule of Assessments. The eGFR calculation is based on the Chronic Kidney Disease Epidemiology Collaboration (CKD-EPI) formula for all participants using serum creatinine collected prior to study drug administration, if applicable. For the determination of CRR and PRR at Week 26 and Week 50, 2 serum creatinine samples are obtained within 2 weeks prior to each of these study visits. The change from baseline in eGFR is measured throughout the course of the study. In addition, the slope of eGFR is computed through Week 26 and Week 50 for participants in the IgAN cohort.

For participants in both disease cohorts, hematuria from spot urine samples is evaluated to assess the effect of ravulizumab on disease course. The degree of hematuria is assessed by examination of the spun urine sediment by microscopy (RBC/hpf). Single void collections for random spot urine sample for hematuria evaluation are collected. If the Investigator determines that the hematuria is transient due to menses in women or exercise, the sample may need to be repeated. Random spot urine samples for hematuria measurement are collected throughout the study as outlined in the Schedule of Assessments and are analyzed by a central laboratory. On dosing days, samples are collected prior to study drug administration, if applicable.

The local hematuria evaluation by microscopy or urinary dipstick is utilized to determine eligibility for the study at Screening for participants with IgAN.

13. Biomarkers

Blood (whole blood, serum & plasma) samples for biomarker research are collected from all participants at the time points specified in the Schedule of Assessments. Biomarkers measured include, but are not limited to, assessments of the following: Complement pathway dysregulation (e.g., soluble C5b-9 [sC5b-9], Factor Ba, Factor Bb, C5a, etc.)

Urine samples for biomarker research are collected from all participants at the time points specified in the Schedule of Assessments. Biomarkers measured include, but are not limited to, assessments of the following: complement pathway dysregulation (e.g., sC5b-9, Factor Ba, Factor Bb, C5a, etc.), renal injury biomarkers (e.g., CD163, MCP-1, EGF, etc.), and creatinine.

Kidney tissue biopsies are stained for the presence of biomarkers which provide clinical evidence of the disease pathophysiology and response to treatment (e.g., C5b-9, C3c, C3, C4d, CD68, properdin, complement component 9 [C9], C1q, C5aR, etc.). For participants in the LN cohort who undergo repeated kidney biopsy(s) during the study, the LN classification is assessed.

Residual blood, urine, and biopsy samples from exploratory biomarkers, PK, PD, immunogenicity, re stored for additional method developments of assays (e.g., prognostics and/or companion diagnostics related to the study drug target, disease process, pathways associated with disease state, other complement-related diseases, and/or mechanism of action of ravulizumab). Samples are retained to enable further analysis on ravulizumab continues but no longer than 5 years after termination of the study or other period as per local requirements.

14. Other Exploratory Assessments

Autoantibodies are assessed for the LN Cohort. Blood samples are collected for anti-dsDNA and anti-C1q) autoantibodies during Screening and according to the Schedule of Assessments through the end of the Extension Period (Day 351).

SLEDAI-2K is assessed for the LN Cohort. The SLEDAI-2K tool assesses disease activity across 24 disease descriptors. The total score ranges from 0 to 105, with higher scores representing more significant degrees of disease activity. The SLEDAI-2K assessment is used for the determination of Extrarenal SLE Flare which is defined as an increase in SLEDAI-2K ≥4 points that is not accounted for by proteinuria, hematuria, urinary casts, hypocomplementemia, pyuria, or an increase in anti-dsDNA antibody level.

Blood and urine samples are collected (at selected study sites only) for exploratory Real Time Complement Activity (RTCA) during Screening and according to the Schedule of Assessments through the end of the Initial Evaluation Period (Day 183). The RTCA analysis is performed at clinical sites using freshly collected whole blood dipotassium ethylenediaminetetraacetic acid (K2EDTA) and urine samples. Blood and urine samples for RTCA are collected prior to administration of study drug on dosing days, if applicable. The results are de-identified using the participant study ID number and all site personnel are blinded from the RTCA results.

Quality of life scales will be administered electronically by the Investigator or a qualified site staff prior to other study procedures at visits specified in the Schedule of Assessments. Participants in both cohorts have the following validated quality of life scales administered.

The Short Form (36) (SF-36v2) Health Survey is used to assess the participant's quality of life. In the SF-36v2 Questionnaire, participants are instructed to rate their health and capacity to perform activities of daily living in 8 domains including physical functioning, physical role limitations, bodily pain, general health, vitality, social functioning, emotional role limitations, and mental health during the last 4 weeks. Raw domain scores are determined and transformed to a 0 to 100 scale as described in the SF-36v2 manual. Domains are scored from 0 to 100 with lower scores indicating increased disability.

The EuroQoL 5-Dimensions 5-Level (EQ-5D-5L) is a self-assessed, standardized instrument to measure health related quality of life and has been used in a wide range of health conditions. The EQ 5D 5L is a 5 scale participant reported outcome tool measuring pain/discomfort, mobility, self-care, usual activities and anxiety/depression.

Participants in the LN cohort also have the following validated quality of life scales administered: The Functional Assessment of Chronic Illness Therapy (FACIT) Fatigue scale, Version 4.0, is a 13 item questionnaire that assesses self reported fatigue and its impact upon daily activities and function over the preceding 7 days.

Antidrug antibodies to ravulizumab (i.e., antidrug antibody) are evaluated in serum samples collected from all participants according to the Schedule of Assessments. Additionally, serum samples are also collected at the final visit from participants who discontinued the study drug or were withdrawn from the study.

Serum samples are screened for antibodies binding to ravulizumab and the titer of confirmed positive samples is reported. Other analyses can be performed to further characterize the immunogenicity of ravulizumab.

The detection and characterization of antibodies to ravulizumab is performed using a validated assay method. Samples collected for detection of antibodies to ravulizumab are also evaluated for ravulizumab serum concentration to enable interpretation of the antibody data. Confirmed antibody positive samples can be further evaluated for antibody titer and the presence of neutralizing antibodies.

15. Statistical Considerations

The primary hypothesis for this study is that ravulizumab is superior to placebo in decreasing proteinuria. Hypothesis testing will be one-sided and performed at the 0.05 level of significance.

This study enrolls 60 participants in both the IgAN and LN cohorts in a 2:1 ratio to ravulizumab and placebo, for a total of 120 participants. Sample size calculations are based on a one-sided two-sample t-test of log-transformed proteinuria values.

The following populations set forth in Table 10 are defined for this study:

TABLE 10
Populations for Analyses

| Population | Description |
|---|---|
| Randomized Set | All randomized participants. Participants are |
| | analyzed as randomized for reporting disposition, |
| | demographics, and baseline characteristics. |
| Full Analysis | All randomized participants who receive at least 1 |
| Set (FAS) | dose of study drug. Participants are analyzed as |
| | randomized for reporting efficacy data. |
| Modified Full | The mF AS is a subset of the FAS, excluding |
| Analysis | participants who, for reasons related to emergency |
| Set (mFAS) | (e.g., quarantine, travel restrictions), received a dose |
| | of ravulizumab ≥28 days after the scheduled dosing |
| | time point or missed a dose altogether. |
| Safety Set | All participants who receive at least 1 dose of study |
| | drug. Participants are analyzed according to the |
| | study drug they actually received for reporting |
| | exposure and safety data. |
| Per Protocol | All randomized participants who receive at least 1 |
| Set | dose of study drug and without major protocol |
| | deviations. |
| Pharmacokinetic/ | All participants who receive at least 1 dose of study |
| Pharmacodynamic | drug and who have evaluable PK/PD data. |
| (PK/PD) Analysis | |
| Set | |

Summary statistics are computed and displayed by treatment group and by visit, where applicable. Descriptive statistics for continuous variables minimally include the number of participants, mean, SD, minimum, median, and maximum. For categorical variables, frequencies, and percentages are presented. Graphical displays are provided as appropriate. Analyses are performed using the SAS® software Version 9.4 or higher.

The analyses for participants in the LN cohort and participants with IgAN cohort is conducted and reported separately. Participants in each disease-specific cohort are analyzed as randomized, regardless of actual treatment received.

The primary analysis of the primary efficacy endpoint is based on the Full Analysis Set (FAS).

For the LN cohort, proteinuria is measured by UPCR in g/g derived from a single 24-hour urine collection at Screening and the mean of 2 separate 24-hour urine collections at Week 26.

For the IgAN cohort, proteinuria is measured by absolute protein in g/day derived from the mean of 2 valid 24-hour urine collections.

To reduce skewness, the natural logarithm is used to transform proteinuria values before analysis. A mixed-effect model for repeated-measures (MMRM) is used for the primary efficacy endpoint using all available longitudinal data (either complete or partial). The model includes change from baseline in log-transformed proteinuria as the response variable and fixed, categorical effects of treatment group, visit, and treatment group by visit interaction as well as a fixed, continuous effect of baseline log proteinuria as a covariate. An unstructured covariance matrix is used to model the correlations among repeated measurements within each participant. If this analysis fails to converge, a first-order autoregressive covariance matrix is used. The Kenward-Roger approximation is used to estimate denominator degrees of freedom. The treatment effect is evaluated using a contrast for treatment group-by-visit term at Week 26. The point estimate and two-sided 90% confidence interval (CI) for the mean difference of log-transformed proteinuria is back-transformed (via exponentiation) to obtain the GMR and corresponding two-sided 90% CI. The values are then expressed as percentage change in adjusted geometric mean of proteinuria at Week 26 relative to baseline.

Participants in the LN cohort who receive Rescue Therapy for a protocol-defined Renal Flare are evaluated up to the point of Rescue Therapy only for the primary efficacy analysis in order to evaluate the effect of the assigned treatment only. Additional sensitivity analyses are performed to assess the impact of the missing data and assumptions.

For the IgAN cohort, participants initially randomized to the placebo group receive ravulizumab in the Extension Period. Therefore, analysis of the secondary endpoints during the Extension Period is summarized separately for each treatment group and baseline for the placebo group is re-defined as the last measurement taken before the first dose of ravulizumab during the Extension Period (i.e., the Week 26 measurement). The primary efficacy endpoint analysis is also performed on the Per Protocol Set.

The secondary efficacy analyses are descriptive in nature and based on the FAS. For the analysis of the secondary endpoints, only data up to the point of Rescue Therapy for participants in the LN cohort who receive Rescue Therapy for protocol-defined Renal Flare is included. Additional sensitivity analyses are conducted to evaluate the robustness of missing data assumptions.

Secondary Efficacy Analyses for Both LN Cohort and IgAN Cohort are as follows. The percent change from baseline in proteinuria at Week 50 is analyzed in a similar manner as the primary endpoint, except the contrast used in the MMRM analysis will be for treatment group-by-visit term at Week 50. The percentage of participants with >30% and >50% reduction in proteinuria at Week 26 and Week 50 is summarized by treatment group by calculating the point estimate and two-sided 90% CI, based on exact confidence limits using the Clopper Pearson method. The following endpoints are summarized at baseline and each postbaseline time point by treatment group using descriptive statistics for the observed value as well as the change from baseline: estimated glomerular filtration rate (eGFR) and serum C3 and C4 concentrations

The following secondary endpoints re summarized by treatment group by calculating the point estimate and two-sided 90% CI, based on exact confidence limits using the Clopper-Pearson method: (1) percentage of participants meeting the criteria for CRR as well as individual components of CRR at Week 26 and Week 50, (2) percentage of participants meeting the criteria for PRR at Week 26 and Week 50, (3) percentage of participants with successful corticosteroid taper at Week 14, Week 26, and Week 50, (4) percentage of participants with protocol-defined Renal Flare through Week 50, (5) percentage of participants with protocol-defined Severe Extrarenal SLE Flare through Week 50, and (6) percentage of participants with Treatment Failure through Week 50.

Time to UPCR ≤0.5 g/g is summarized based on spot urine samples. A Kaplan-Meier cumulative distribution curve is generated for treatment group, and a log-rank test comparing the curves is performed. The corresponding summary table presents by treatment group the cumulative distribution function (CDF) estimate, the number of participants at risk, the number of participants responding, and the number of participants censored at each postbaseline time point. The table also present the first quartile, median, and third quartile, along with two-sided 90% CI, of time to UPCR ≤0.5 g/g. Serum albumin is summarized at baseline and each postbaseline time point by treatment group using descriptive statistics for the observed value as well as the change from baseline.

Secondary Efficacy Analyses for IgAN Cohort: the percentage of participants meeting the criteria for Partial Remission at Week 26 and Week 50 is summarized by treatment group by calculating the point estimate and two-sided 90% CI, based on exact confidence limits using the Clopper-Pearson method.

The secondary efficacy analyses re descriptive in nature and no adjustment for multiplicity will be performed.

All safety analyses re performed on the Safety Set and are based on the actual treatment received.

A treatment-emergent adverse event (TEAE) is any adverse event that starts during or after the first dose of study drug. Adverse events that start 56 days or later after the last dose of study drug will not be considered as treatment emergent. A treatment-emergent SAE (TESAE) is a TEAE that is serious. The incidence of TEAEs, TEAEs leading to withdrawal from the study, TEAEs leading to study drug discontinuation, drug-related TEAEs, and TESAEs is summarized by treatment group for each disease cohort separately. All adverse events are coded using MedDRA version 23.0 or higher and are summarized by System Organ Class (SOC) and Preferred Term overall, by severity, and by relationship to study drug.

Adverse changes from baseline in physical examination findings are classified as AEs and analyzed accordingly. Vital signs are summarized descriptively by treatment group at baseline and postbaseline time points and for changes from baseline separately for each disease cohort.

Observed values and changes from baseline in clinical chemistry, hematology, and urinalysis are summarized descriptively by treatment group at baseline and at each postbaseline time point separately for each disease cohort. For laboratory results that can be classified as normal, low or high based on normal range values, shifts from baseline in classification are summarized for all study visits.

By-participant data listings of electrocardiogram (ECG) parameters are provided separately for each disease cohort. Electrocardiograms are evaluated and summarized as normal, abnormal not clinically significant, or abnormal clinically significant. A shift from baseline to worst on-study ECG table is presented for ECG results. Observed values and change from baseline in ECG intervals (PR, RR, QT, and QTc) are summarized descriptively at baseline and each postbaseline time point. The QT interval is corrected for heart rate using Fridericia's formula (QTcF).

For pharmacokinetic (PK)/pharmacodynamic (PD) analyses, graphs of mean serum ravulizumab concentration-time profiles are constructed. Graphs of serum concentration-time profiles for individual participants can also be provided. Actual dose administration and sampling times are used for all calculations. Descriptive statistics are calculated for serum concentration data at each sampling time, as appropriate. The PD effects of ravulizumab are evaluated by assessing the absolute values and changes and percentage changes from baseline in serum free C5 concentrations over time, as appropriate. Descriptive statistics are calculated for the PD data at each sampling time, as appropriate.

The incidence and titers for ADAs to ravulizumab are presented at each postbaseline time point in tabular format separately for each disease cohort. Additionally, any confirmed ADA positive samples are tested for the presence of neutralizing antibodies to ravulizumab.

The exploratory efficacy analyses are descriptive in nature and based on the FAS. For continuous endpoints, data is summarized at baseline and each postbaseline time point by treatment group using descriptive statistics for the observed value, as well as the change from baseline. For categorical endpoints, data is summarized by treatment group by calculating the point estimate and two-sided 90% CI, based on exact confidence limits using the Clopper-Pearson method.

For the LN cohort, time to CRR, time to PRR, and time to UPCR>50% decrease from baseline is summarized using spot urine samples. Participants are assigned as responders at the time of their CRR, PRR, or UPCR>50% decrease from baseline, respectively, or censored at the earliest of their discontinuation time, receipt of rescue therapy, or at Week 50 if they have not responded or received rescue therapy by then. Kaplan-Meier cumulative distribution curves are generated for each treatment group, and a log-rank test comparing the curves is performed. A corresponding summary table presents the CDF estimate, the number of participants at risk, the number of participants responding, and the number of participants censored at each postbaseline time point by treatment group. The table also presents first quartile, median, and third quartile, along with corresponding 2-sided 90% CI, of time to response.

Slope of eGFR for the IgAN cohort is computed using all available assessments up to Week 26 or Week 50. The eGFR slope is estimated using a simple linear regression for each participant with eGFR as the dependent variable and time as the independent variable and the mean slope (mL/min/1.73 m2 per year) will be summarized descriptively by treatment group.

The following quality of life assessments are summarized by treatment group at baseline and each postbaseline time point using descriptive statistics for continuous variables for the observed value as well as the change from baseline: EQ-5D-5L, SF-36 total score, and FACIT-Fatigue (preferably for LN cohort).

To ensure the adequacy of the dose regimen, an interim PK/PD analysis for dose confirmation is conducted by an independent clinical pharmacologist. The interim PK confirmation analysis is conducted using masked PK/PD data from the first 10 participants treated with ravulizumab (a minimum of 3 participants in each disease-specific cohort), using data cut when the tenth participant reaches 2 weeks post first dose (i.e., at Day 15). The PK dataset for review includes: (1) Day 1 Cₘₐₓ, Day 15 C_{trough}, and Cₘₐₓ for all 10 participants, (2) Pharmacokinetics data beyond Day 15 Cₘₐₓ timepoint (e.g., Day 29 PK) may be included in the dataset (availability depending upon the enrollment rate), and (3) free and total C5 data associated with above timepoints and ADA data is included in the dataset as supportive evidence. If observed Day 1 Cₘₐₓ, Day 15 Cₘₐₓ and C_{trough} values, and other available PK/PD data are within the expected range, the study proceeds unchanged. If dose regimen adjustment is needed, enrollment is paused until a new regimen is determined. In the event of dose adjustments, the participants treated with the previous dose switch over to the new dose and continue treatment on study but, are excluded from the primary efficacy analysis. Replacement participants can be enrolled to preserve study power.

The primary efficacy analysis is performed for each disease-specific cohort at the end of the 26-week Initial Evaluation Period after all participants in the disease-specific cohort have completed or withdrawn from the 26-week Initial Evaluation Period. This analysis allows for evaluation of the primary endpoint and Phase 3 planning and has no impact on the progression of this study.

In addition, an early interim analysis may be conducted for the IgAN disease cohort (based on feasibility) when at least 50% of participants have been randomly assigned to study treatment and have had the opportunity to complete the 26-week Initial Evaluation Period. This interim analysis, if performed, is conducted by a separate unblinded team and is for Phase 3 planning purposes only with no impact on the progression of the study.

An interim efficacy analysis is performed for each disease-specific cohort at the end of the 50-week Extension Period after all participants in the disease-specific cohort have completed or withdrawn from the 50-week Extension Period.

The final study analysis is conducted at the end of the study.

16. Adverse Events

An adverse event (AE) is any untoward medical occurrence in a participant, temporally associated with the use of study intervention, whether or not considered related to the study intervention. An AE can therefore be any unfavorable and unintended sign (including an abnormal laboratory finding), symptom, or disease (new or exacerbated) temporally associated with the use of study intervention.

The following events meet the AE definition:
1. Any abnormal laboratory test results (hematology, clinical chemistry, or urinalysis) or other safety assessments (e.g., ECG, radiological scans, vital signs measurements), including those that worsen from baseline, considered clinically significant in the medical and scientific judgment of the Investigator (i.e., not related to progression of underlying disease).
2. Exacerbation of a chronic or intermittent pre-existing condition including either an increase in frequency and/or intensity of the condition.
3. New conditions detected or diagnosed after study intervention administration even though it may have been present before the start of the study.
4. Signs, symptoms, or the clinical sequelae of a suspected drug-drug interaction.
5. Signs, symptoms, or the clinical sequelae of a suspected overdose of either study intervention or a concomitant medication. Overdose per se will not be reported as an AE/SAE unless it is an intentional overdose taken with possible suicidal/self-harming intent. Such overdoses should be reported regardless of sequelae.
6. “Lack of efficacy” or “failure of expected pharmacological action” per se is not reported as an AE or SAE. Such instances are captured in the efficacy assessments. However, the signs, symptoms, and/or clinical sequelae resulting from lack of efficacy are reported as AE or SAE if they fulfil the definition of an AE or SAE.

The following events do not meet the AE definition:
1. Medical or surgical procedure (e.g., endoscopy, appendectomy): The condition that leads to the procedure is the AE. Situations in which an untoward medical occurrence did not occur (e.g., hospitalization for elective surgery if planned before the signing the ICF, admissions for social reasons or for convenience).
2. Anticipated day-to-day fluctuations of pre-existing disease(s) or condition(s) present or detected at the start of the study that do not worsen.
3. A medication error (including intentional misuse, abuse, and overdose of the product) or use other than what is defined in the protocol is not considered an AE unless there is an untoward medical occurrence as a result of a medication error.
4. Cases of pregnancy that occur during maternal or paternal exposure to study drug are to be reported within 24 hours of Investigator/site awareness. Data on fetal outcome and breastfeeding is collected for regulatory reporting and safety evaluation.
5. Any clinically significant abnormal laboratory findings or other abnormal safety assessments which are associated with the underlying disease, unless judged by the Investigator to be more severe than expected for the participant's condition.
6. The disease/disorder being studied or expected progression, signs, or symptoms of the disease/disorder being studied, unless more severe than expected for the participant's condition.
7. Situations in which an untoward medical occurrence did not occur (social and/or convenience admission to a hospital).

If an event is not an AE per definition above, then it cannot be an serious adverse event (SAE) even if serious conditions are met (e.g., hospitalization for signs/symptoms of the disease under study, death due to progression of disease). The definition of a SAE is set forth in Table 11.

TABLE 11
SAE Definition
An SAE is defined as any untoward medical occurrence that, at any dose:

1. Results in death
2. Is life-threatening
The term “life-threatening” in the definition of “serious” refers to an event in which the
participant was at risk of death at the time of the event. It does not refer to an event, which
hypothetically might have caused death, if it was more severe.
3. Requires inpatient hospitalization or prolongation of existing hospitalization
In general, hospitalization signifies that the participant has been detained (usually
involving at least an overnight stay) at the hospital or emergency ward for observation
and/or treatment that would not have been appropriate in the physician's office or
outpatient setting. Complications that occur during hospitalization are AEs. If a
complication prolongs hospitalization or fulfills any other serious criteria, the event is
serious. When in doubt as to whether “hospitalization” occurred or was necessary, the AE
is considered serious.
Hospitalization for elective treatment of a pre-existing condition that did not worsen from
baseline is not considered an AE.
4. Results in persistent disability/incapacity
The term disability means a substantial disruption of a person's ability to conduct
normal life functions.
This definition is not intended to include experiences of relatively minor medical
significance such as uncomplicated headache, nausea, vomiting, diarrhea, influenza,
and accidental trauma (e.g., sprained ankle) which may interfere with or prevent
everyday life functions but do not constitute a substantial disruption.
5. Is a congenital anomaly/birth defect
6. Other situations:
Medical or scientific judgment should be exercised in deciding whether SAE reporting
is appropriate in other situations such as important medical events that may not be
immediately life-threatening or result in death or hospitalization but may jeopardize
the participant or may require medical or surgical intervention to prevent one of the
other outcomes listed in the above definition. These events should usually be
considered serious.
Examples of such events include invasive or malignant cancers, intensive treatment in
an emergency room or at home for allergic bronchospasm, blood dyscrasias or
convulsions that do not result in hospitalization, or development of drug dependency
or drug abuse.

A suspected unexpected serious adverse reaction (SUSAR) is defined as a serious event that is not listed in the Investigator's Brochure and that the Investigator identifies as related to investigational product or procedure. United States Title 21 Code of Federal Regulations (CFR) 312.32 and European Union Clinical Trial Directive 2001/20/EC and the associated detailed guidances or national regulatory requirements in participating countries require the reporting of SUSARs. Suspected unexpected serious adverse reactions are reported to the national competent authority and IRBs/IECs where applicable.

The Investigator makes an assessment of intensity for each AE and SAE reported during the study and assign it to one of the following categories from National Cancer Institute Common Terminology Criteria for Adverse Events (CTCAE) v5.0, published 27 Nov. 2017:
1. Grade 1: Mild (awareness of sign or symptom, but easily tolerated)
2. Grade 2: Moderate (discomfort sufficient to cause interference with normal activities)
3. Grade 3: Severe (incapacitating, with inability to perform normal activities)
4. Grade 4: Life-threatening
5. Grade 5: Fatal

An event is defined as “serious” when it meets at least one of the predefined outcomes as described in the definition of an SAE, not when it is rated as severe.

17. Clinical Laboratory Tests

Observed values and changes from baseline in clinical chemistry, hematology, and urinalysis are summarized descriptively by treatment group at baseline and at each postbaseline time point separately for each disease cohort. For laboratory results that can be classified as normal, low or high based on normal range values, shifts from baseline in classification are summarized for all study visits.

The tests set forth in Table 12 are performed by a study central laboratory. Local laboratory results are only required in the event that the central laboratory results are not available in time for either study intervention administration and/or response evaluation. If a local sample is required, it is important that the sample for central analysis is obtained at the same time.

Women of childbearing potential should only be enrolled after a negative serum pregnancy test result at Screening. Additional urine pregnancy testing is standard for the protocol unless serum testing is required by site policies, local regulation, or IRB/IEC and is performed per the time points specified in the Schedule of Activities.

TABLE 12
Protocol-Required Laboratory Assessments

| Laboratory | |
|---|---|
| Assessments | Parameters |
| Hematology | Red blood cell count |
| | Hemoglobin |
| | Hematocrit |
| | Erythrocytes |
| | RBC indices |
| | Mean corpuscular volume |
| | Mean corpuscular hemoglobin |
| | Percentage of reticulocytes |
| | Corpuscular hemoglobin content |
| | White blood cell count with differential |
| | (including early progenitors): |
| | Neutrophils, segmented |
| | Lymphocytes |
| | Monocytes |
| | Eosinophils |
| | Basophils |
| | Platelet count |
| | Mean platelet volume |
| Coagulation panel | INR |
| | PT |
| | APTT |
| | D-Dimer |
| | Fibrinogen |
| Clinical chemistry | Liver function tests: |
| | ALT |
| | AST |
| | ALP |
| | Albumin |
| | Total protein |
| | Bilirubin (total, direct and indirect) |
| | GGT |
| | Glucose (fasting) |
| | Renal function: |
| | Blood urea nitrogen |
| | Calcium |
| | Chloride |
| | Creatinine and eGFR calculated using CKD-EPI formula |
| | Magnesium |
| | Phosphate |
| | Potassium |
| | Sodium |
| | Total carbon dioxide |
| | Urea |
| 24-h urine | Total protein, total creatinine, total albumin, total sodium, |
| | creatinine clearance, and protein to creatinine ratio, albumin to |
| | creatinine ratio. |
| Spot urine studies | Protein, albumin, creatinine, and protein to creatinine and |
| | albumin/creatinine ratio |
| Routine urinalysis | Albumin |
| and urine sediment | Bilirubin |
| | Blood |
| | Erythrocytes |
| | Glucose |
| | Ketones |
| | Leukocyte esterase |
| | Nitrite |
| | pH |
| | Protein |
| | Specific gravity |
| | Urobilinogen |
| | Urine sediment: number of RBCs/high-power field and number |
| | of RBC casts |
| PK/PD and | Serum PK |
| immunogenicity | Serum PD (free and total C5) |
| | Immunogenicity (ADA) |
| Other study-specific | HCV and HBV PCR viral load |
| tests | HIV-1 and HIV-2 antibody |
| | Serum follicle-stimulating hormone and estradiol (as needed in |
| | women of non-childbearing potential only) |
| | Serum or urine human chorionic gonadotropin pregnancy test |
| | (as needed for WOCBP)^{a} |
| | Complement: C3, C4, and CH50 |
| | Autoantibody profile: ANA, anti-dsDNA, anti-Sm, anti-RNP, |
| | anti-Ro, anti-La, anti-C1q, anti-phospholipid antibodies (LN |
| | cohort only) |
| | Anti-ds-DNA antibody: to be measured by ELISA at all visits as |
| | part of SLEDAI Assessment (LN cohort only) |

^{a}Serum pregnancy test at Screening and End of Study Visit/Early Discontinuation Visit, and local urine pregnancy test at all other times as specified in Schedule of Assessments
Abbreviations: ADA = antidrug antibody; ALP = alkaline phosphatase; ALT = alanine aminotransferase; ANA = antinuclear antibody; anti-C1q = anti-complement component C1q; anti-La = anti-small RNA binding exonuclease protection factor La; anti-Ro = anti-Sjögren's-syndrome-related antigen A; anti-Sm = anti-Smith antibody; APTT = activated partial thromboplastin time; AST = aspartate aminotransferase; C3, C4, and C5 = complement components 3, 4, and 5; CH50 = 50% hemolytic complement activity; CKD-EPI = Chronic Kidney Disease Epidemiology Collaboration; dsDNA = double-stranded DNA; eGFR = estimated glomerular filtration rate; ELISA = enzyme-linked immunosorbent assay; GGT = gamma-glutamyltransferase; HBV = hepatitis B virus; HCV = hepatitis C virus; INR = international normalized ratio; LN = lupus nephritis; RNP = ribonucleoprotein; PCR = polymerase chain reaction; PD = pharmacodynamic; PK = pharmacokinetic; PT = prothrombin time; RBC = red blood cell; SLEDAI = Systemic Lupus Erythematosus Disease Activity Index; WOCBP = women of childbearing potential.

SEQUENCE SUMMARY

SEQ ID NO: 1
GYIFSNYWIQ
SEQ ID NO: 2
EILPGSGSTEYTENFKD
SEQ ID NO: 3
YFFGSSPNWYFDV
SEQ ID NO: 4
GASENIYGALN
SEQ ID NO: 5
GATNLAD
SEQ ID NO: 6
QNVLNTPLT
SEQ ID NO: 7
QVQLVQSGAE VKKPGASVKV SCKASGYIFS NYWIQWVRQA PGQGLEWMGE
ILPGSGSTEY TENFKDRVTM TRDTSTSTVY MELSSLRSED TAVYYCARYF
FGSSPNWYFD VWGQGTLVTV SS
SEQ ID NO: 8
DIQMTQSPSS LSASVGDRVT ITCGASENIY GALNWYQQKP GKAPKLLIYG
ATNLADGVPS RFSGSGSGTD FTLTISSLQP EDFATYYCQN VLNTPLTFGQ
GTKVEIK
SEQ ID NO: 9
ASTKGPSVFP LAPCSRSTSE STAALGCLVK DYFPEPVTVS WNSGALTSGV
HTFPAVLQSS GLYSLSSVVT VPSSNFGTQT YTCNVDHKPS NTKVDKTVER
KCCVECPPCP APPVAGPSVF LFPPKPKDTL MISRTPEVTC VVVDVSQEDP
EVQFNWYVDG VEVHNAKTKP REEQFNSTYR VVSVLTVLHQ DWLNGKEYKC
KVSNKGLPSS IEKTISKAKG QPREPQVYTL PPSQEEMTKN QVSLTCLVKG
FYPSDIAVEW ESNGQPENNY KTTPPVLDSD GSFFLYSRLT VDKSRWQEGN
VFSCSVMHEA LHNHYTQKSL SLSLGK
SEQ ID NO: 10
QVQLVQSGAE VKKPGASVKV SCKASGYIFS NYWIQWVRQA PGQGLEWMGE
ILPGSGSTEY TENFKDRVTM TRDTSTSTVY MELSSLRSED TAVYYCARYF
FGSSPNWYFD VWGQGTLVTV SSASTKGPSV FPLAPCSRST SESTAALGCL
VKDYFPEPVT VSWNSGALTS GVHTFPAVLQ SSGLYSLSSV VTVPSSNFGT
QTYTCNVDHK PSNTKVDKTV ERKCCVECPP CPAPPVAGPS VFLFPPKPKD
TLMISRTPEV TCVVVDVSQE DPEVQFNWYV DGVEVHNAKT KPREEQFNST
YRVVSVLTVL HQDWLNGKEY KCKVSNKGLP SSIEKTISKA KGQPREPQVY
TLPPSQEEMT NQVSLTCLVK GFYPSDIAVE WESNGQPENN YKTTPPVLDS
DGSFFLYSRL TVDKSRWQEG NVFSCSVMHE ALHNHYTQKS LSLSLGK
SEQ ID NO: 11
DIQMTQSPSS LSASVGDRVT ITCGASENIY GALNWYQQKP GKAPKLLIYG
ATNLADGVPS RFSGSGSGTD FTLTISSLQP EDFATYYCQN VLNTPLTFGQ
GTKVEIKRTV AAPSVFIFPP SDEQLKSGTA SVVCLLNNFY PREAKVQWKV
DNALQSGNSQ ESVTEQDSKD STYSLSSTLT LSKADYEKHK VYACEVTHQG
LSSPVTKSFN RGEC
SEQ ID NO: 12
QVQLVQSGAE VKKPGASVKV SCKASGHIFS NYWIQWVRQA PGQGLEWMGE
ILPGSGHTEY TENFKDRVTM TRDTSTSTVY MELSSLRSED TAVYYCARYF
FGSSPNWYFD VWGQGTLVTV SS
SEQ ID NO: 13
ASTKGPSVFP LAPCSRSTSE STAALGCLVK DYFPEPVTVS WNSGALTSGV
HTFPAVLQSS GLYSLSSVVT VPSSNFGTQT YTCNVDHKPS NTKVDKTVER
KCCVECPPCP APPVAGPSVF LFPPKPKDTL MISRTPEVTC VVVDVSQEDP
EVQFNWYVDG VEVHNAKTKP REEQFNSTYR VVSVLTVLHQ DWLNGKEYKC
KVSNKGLPSS IEKTISKAKG QPREPQVYTL PPSQEEMTKN QVSLTCLVKG
FYPSDIAVEW ESNGQPENNY KTTPPVLDSD GSFFLYSRLT VDKSRWQEGN
VFSCSVLHEA LHSHYTQKSL SLSLGK
SEQ ID NO: 14
QVQLVQSGAE VKKPGASVKV SCKASGHIFS NYWIQWVRQA PGQGLEWMGE
ILPGSGHTEY TENFKDRVTM TRDTSTSTVY MELSSLRSED TAVYYCARYF
FGSSPNWYFD VWGQGTLVTV SSASTKGPSV FPLAPCSRST SESTAALGCL
VKDYFPEPVT VSWNSGALTS GVHTFPAVLQ SSGLYSLSSV VTVPSSNFGT
QTYTCNVDHK PSNTKVDKTV ERKCCVECPP CPAPPVAGPS VFLFPPKPKD
TLMISRTPEV TCVVVDVSQE DPEVQFNWYV DGVEVHNAKT KPREEQFNST
YRVVSVLTVL HQDWLNGKEY KCKVSNKGLP SSIEKTISKA KGQPREPQVY
TLPPSQEEMT KNQVSLTCLV KGFYPSDIAV EWESNGQPEN NYKTTPPVLD
SDGSFFLYSR LTVDKSRWQE GNVFSCSVLH EALHSHYTQK SLSLSLGK
SEQ ID NO: 15
ASTKGPSVFP LAPCSRSTSE STAALGCLVK DYFPEPVTVS WNSGALTSGV
HTFPAVLQSS GLYSLSSVVT VTSSNFGTQT YTCNVDHKPS NTKVDKTVER
KCCVECPPCP APPVAGPSVF LFPPKPKDTL YITREPEVTC VVVDVSHEDP
EVQFNWYVDG MEVHNAKTKP REEQFNSTFR VVSVLTVVHQ DWLNGKEYKC
KVSNKGLPAP IEKTISKTKG QPREPQVYTL PPSREEMTKN QVSLTCLVKG
FYPSDIAVEW ESNGQPENNY KTTPPMLDSD GSFFLYSKLT VDKSRWQQGN
VFSCSVMHEA LHNHYTQKSL SLSPG
SEQ ID NO: 16
QVQLVQSGAE VKKPGASVKV SCKASGYIFS NYWIQWVRQA PGQGLEWMGE
ILPGSGSTEY TENFKDRVTM TRDTSTSTVY MELSSLRSED TAVYYCARYF
FGSSPNWYFD VWGQGTLVTV SSASTKGPSV FPLAPCSRST SESTAALGCL
VKDYFPEPVT VSWNSGALTS GVHTFPAVLQ SSGLYSLSSV VTVTSSNFGT
QTYTCNVDHK PSNTKVDKTV ERKCCVECPP CPAPPVAGPS VFLFPPKPKD
TLYITREPEV TCVVVDVSHE DPEVQFNWYV DGMEVHNAKT KPREEQFNST
FRVVSVLTVV HQDWLNGKEY KCKVSNKGLP APIEKTISKT KGQPREPQVY
TLPPSREEMT KNQVSLTCLV KGFYPSDIAV EWESNGQPEN NYKTTPPMLD
SDGSFFLYSK LTVDKSRWQQ GNVFSCSVMH EALHNHYTQK SLSLSPGK
SEQ ID NO: 17
GASENIYHALN
SEQ ID NO: 18
EILPGSGHTEYTENFKD
SEQ ID NO: 19
GHIFSNYWIQ
SEQ ID NO: 20
QVQLVQSGAE VKKPGASVKV SCKASGHIFS NYWIQWVRQA PGQGLEWMGE
ILPGSGHTEY TENFKDRVTM TRDTSTSTVY MELSSLRSED TAVYYCARYF
FGSSPNWYFD VWGQGTLVTV SSASTKGPSV FPLAPCSRST SESTAALGCL
VKDYFPEPVT VSWNSGALTS GVHTFPAVLQ SSGLYSLSSV VTVPSSNFGT
QTYTCNVDHK PSNTKVDKTV ERKCCVECPP CPAPPVAGPS VFLFPPKPKD
TLMISRTPEV TCVVVDVSQE DPEVQFNWYV DGVEVHNAKT KPREEQFNST
YRVVSVLTVL HQDWLNGKEY KCKVSNKGLP SSIEKTISKA KGQPREPQVY
TLPPSQEEMT KNQVSLTCLV KGFYPSDIAV EWESNGQPEN NYKTTPPVLD
SDGSFFLYSR LTVDKSRWQE GNVFSCSVMH EALHNHYTQK SLSLSLGK
SEQ ID NO: 21
SYAIS
SEQ ID NO: 22
GIGPFFGTANYAQKFQG
SEQ ID NO: 23
DTPYFDY
SEQ ID NO: 24
SGDSIPNYYVY
SEQ ID NO: 25
DDSNRPS
SEQ ID NO: 26
QSFDSSLNAEV
SEQ ID NO: 27
QVQLVQSGAE VKKPGSSVKV SCKASGGTFS SYAISVWRQA PGQGLEWMGG
IGPFFGTANY AQKFQGRVTI TADESTSTAY MELSSLRSED TAVYYCARDT
PYFDYWGQGT LVTVSS
SEQ ID NO: 28
DIELTQPPSV SVAPGQTARI SCSGDSIPNY YVYWYQQKPG QAPVLVIYDD
SNRPSGIPER FSGSNSGNTA TLTISGTQAE DEADYYCQSF DSSLNAEVFG
GGTKLTVL
SEQ ID NO: 29
NYIS
SEQ ID NO: 30
IIDPDDSYTEYSPSFQG
SEQ ID NO: 31
YEYGGFDI
SEQ ID NO: 32
SGDNIGNSYVH
SEQ ID NO: 33
KDNDRPS
SEQ ID NO: 34
GTYDIESYV
SEQ ID NO: 35
EVQLVQSGAE VKKPGESLKI SCKGSGYSFT NYISWVRQMP GKGLEWMGII
DPDDSYTEYS PSFQGQVTIS ADKSISTAYL QWSSLKASDT AMYYCARYEY
GGFDIWGQGT LVTVSS
SEQ ID NO: 36
SYELTQPPSV SVAPGQTARI SCSGDNIGNS YVHWYQQKPG QAPVLVIYKD
NDRPSGIPER FSGSNSGNTA TLTISGTQAE DEADYYCGTY DIESYVFGGG
TKLTVL
SEQ ID NO: 37
SSYYVA
SEQ ID NO: 38
AIYTGSGATYKASWAKG
SEQ ID NO: 39
DGGYDYPTHAMHY
SEQ ID NO: 40
QASQNIGSSLA
SEQ ID NO: 41
GASKTHS
SEQ ID NO: 42
QSTKVGSSYGNH
SEQ ID NO: 43
QVQLVESGGG LVQPGGSLRL SCAASGFTSH SSYYVAWVRQ APGKGLEWVG
AIYTGSGATY KASWAKGRFT ISKDTSKNQV VLTMTNMDPV DTATYYCASD
GGYDYPTHAM HYWGQGTLVT VSS
SEQ ID NO: 44
DVVMTQSPSS LSASVGDRVT ITCQASQNIG SSLAWYQQKP GQAPRLLIYG
ASKTHSGVPSRFSGSGSGTD FTLTISSLQP EDVATYYCQS TKVGSSYGNH
FGGGTKVEIK
SEQ ID NO: 45
QVQLVESGGG LVQPGRSLRL SCAASGFTVH SSYYMAWVRQ APGKGLEWVG
AIFTGSGAEY KAEWAKGRVT ISKDTSKNQV VLTMTNMDPV DTATYYCASD
AGYDYPTHAM HYWGQGTLVT VSSASTKGPS VFPLAPSSKS TSGGTAALGC
LVKDYFPEPV TVSWNSGALT SGVHTFPAVL QSSGLYSLSS VVTVPSSSLG
TQTYICNVNH KPSNTKVDKK VEPKSCDKTH TCPPCPAPEL RRGPKVFLFP
PKPKDTLMIS RTPEVTCVVV DVSHEDPEVK FNWYVDGVEV HNAKTKPREE
QYNSTYRVVS VLTVLHQDWL NGKEYKCKVS NKGLPSSIEK TISKAKGQPR
EPQVYTLPPS REEMTKNQVS LTCLVKGFYP SDIAVEWESN GQPENNYKTT
PPVLDSDGSF FLYSKLTVDK SRWQQGNVFS CSVLHEALHA HYTRKELSLS
P
SEQ ID NO: 46
DIQMTQSPSS LSASVGDRVT ITCRASQGIS SSLAWYQQKP GKAPKLLIYG
ASETESGVPS RFSGSGSGTD FTLTISSLQP EDFATYYCQN TKVGSSYGNT
FGGGTKVEIK RTVAAPSVFI FPPSDEQLKS GTASVVCLLN NFYPREAKVQ
WKVDNALQSG NSQESVTEQD SKDSTYSLSS TLTLSKADYE KHKVYACEVT
HQGLSSPVTK SFNRGEC
SEQ ID NO: 47
QVQLQESGPGLVKPSETLSLTCTVSGDSVSSSYWTWIRQPPGKGLEWIGYIYYSGSSN
YNPSLKSRATISVDTSKNQFSLKLSSVTAADTAVYYCAREGNVDTTMIFDYWGQGTLV
TVSS
SEQ ID NO: 48
AIQMTQSPSSLSASVGDRVTITCRASQGIRNDLGWYQQKPGKAPKLLIYAASSLQSGVP
SRFAGRGSGTDFTLTISSLQPEDFATYYCLQDFNYPWTFGQGTKVEIK
SEQ ID NO: 49
QVQLQESGPGLVKPSETLSLTCTVSGDSVSSSYWTWIRQPPGKGLEWIGYIYYSGSSNY
NPSLKSRATISVDTSKNQFSLKLSSVTAADTAVYYCAREGNVDTTMIFDYWGQGTLVTV
SSASTKGPSVFPLAPCSRSTSESTAALGCLVKDYFPEPVTVSWNSGALTSGVHTFPAVL
QSSGLYSLSSVVTVPSSSLGTKTYTCNVDHKPSNTKVDKRVESKYGPPCPPCPAPEFLG
GPSVFLFPPKPKDTLMISRTPEVTCVVVDVSQEDPEVQFNWYVDGVEVHNAKTKPREEQ
FNSTYRVVSVLTVLHQDWLNGKEYKCKVSNKGLPSSIEKTISKAKGQPREPQVYTLPPS
QEEMTKNQVSLTCLVKGFYPSDIAVEWESNGQPENNYKTTPPVLDSDGSFFLYSRLTVD
KSRWQEGNVFSCSVMHEALHNHYTQKSLSLSLGK
SEQ ID NO: 50
AIQMTQSPSSLSASVGDRVTITCRASQGIRNDLGWYQQKPGKAPKLLIYAASSLQSGVP
SRFAGRGSGTDFTLTISSLQPEDFATYYCLQDFNYPWTFGQGTKVEIKRTVAAPSVFIF
PPSDEQLKSGTASVVCLLNNFYPREAKVQWKVDNALQSGNSQESVTEQDSKDSTYSLSS
TLTLSKADYEKHKVYACEVTHQGLSSPVTKSFNRGEC

What is claimed is:

1. A method of treating a human patient with C5-mediated glomerular nephritis (GN), the method comprising administering to the patient an effective amount of an anti-C5 antibody, or antigen binding fragment thereof, comprising CDR1, CDR2 and CDR3 heavy chain sequences as set forth in SEQ ID NOs:19, 18 and 3, respectively, and CDR1, CDR2 and CDR3 light chain sequences as set forth in SEQ ID NOs:4, 5 and 6, respectively, wherein the anti-C5 antibody or antigen binding fragment thereof, is administered:
(a) once on Day 1 at a dose of 2400 mg, followed by a dose of 3000 mg at Week 2 and once every eight weeks thereafter to a patient weighing ≥40 to <60 kg;
(b) once on Day 1 at a dose of 2700 mg, followed by a dose of 3900 mg at Week 2 and once every eight weeks thereafter to a patient weighing ≥60 to <100 kg; or
(c) once on Day 1 at a dose of 3000 mg, followed by a dose of 5400 mg at Week 2 and once every eight weeks thereafter to a patient weighing ≥100 kg.

2. The method of claim 1, further comprising administering the anti C5 antibody, or antigen binding fragment thereof:
(a) once on Day 183 at a dose of 900 mg, followed by a dose of 3000 mg at Day 197 and once every eight weeks thereafter to a patient weighing ≥40 to <60 kg;
(b) once on Day 183 at a dose of 900 mg, followed by a dose of 3900 mg at Day 197 and once every eight weeks thereafter to a patient weighing ≥60 to <100 kg; or
(c) once on Day 183 at a dose of 900 mg, followed by a dose of 5400 mg at Day 197 and once every eight weeks thereafter to a patient weighing ≥100 kg.

3. A method of treating a human patient with lupus nephritis (LN), the method comprising administering to the patient an effective amount of an anti-C5 antibody, or antigen binding fragment thereof, comprising CDR1, CDR2 and CDR3 heavy chain sequences as set forth in SEQ ID NOs:19, 18 and 3, respectively, and CDR1, CDR2 and CDR3 light chain sequences as set forth in SEQ ID NOs:4, 5 and 6, respectively, wherein the anti-C5 antibody or antigen binding fragment thereof, is administered:
(a) once on Day 1 at a dose of 2400 mg, followed by a dose of 3000 mg at Week 2 and once every eight weeks thereafter to a patient weighing ≥40 to <60 kg;
(b) once on Day 1 at a dose of 2700 mg, followed by a dose of 3900 mg at Week 2 and once every eight weeks thereafter to a patient weighing ≥60 to <100 kg; or
(c) once on Day 1 at a dose of 3000 mg, followed by a dose of 5400 mg at Week 2 and once every eight weeks thereafter to a patient weighing ≥100 kg.

4. A method of treating a human patient with immunoglobulin A nephropathy (IgAN), the method comprising administering to the patient an effective amount of an anti-C5 antibody, or antigen binding fragment thereof, comprising CDR1, CDR2 and CDR3 heavy chain sequences as set forth in SEQ ID NOs:19, 18 and 3, respectively, and CDR1, CDR2 and CDR3 light chain sequences as set forth in SEQ ID NOs:4, 5 and 6, respectively, wherein the anti-C5 antibody or antigen binding fragment thereof, is administered:
(a) once on Day 1 at a dose of 2400 mg, followed by a dose of 3000 mg at Week 2 and once every eight weeks thereafter to a patient weighing ≥40 to <60 kg;
(b) once on Day 1 at a dose of 2700 mg, followed by a dose of 3900 mg at Week 2 and once every eight weeks thereafter to a patient weighing ≥60 to <100 kg; or
(c) once on Day 1 at a dose of 3000 mg, followed by a dose of 5400 mg at Week 2 and once every eight weeks thereafter to a patient weighing ≥100 kg.

5. The method of claim 4, further comprising administering the anti C5 antibody, or antigen binding fragment thereof:
(a) once on Day 183 at a dose of 900 mg, followed by a dose of 3000 mg at Day 197 and once every eight weeks thereafter to a patient weighing ≥40 to <60 kg;
(b) once on Day 183 at a dose of 900 mg, followed by a dose of 3900 mg at Day 197 and once every eight weeks thereafter to a patient weighing ≥60 to <100 kg; or
(c) once on Day 183 at a dose of 900 mg, followed by a dose of 5400 mg at Day 197 and once every eight weeks thereafter to a patient weighing ≥100 kg.

6. The method of any one of the preceding claims, wherein the anti-C5 antibody, or antigen binding fragment thereof, further comprises a variant human Fc constant region that binds to human neonatal Fc receptor (FcRn), wherein the variant human Fc constant region comprises Met429Leu and Asn435Ser substitutions at residues corresponding to methionine 428 and asparagine 434 of a native human IgG Fc constant region, each in EU numbering.

7. The method of any one of the preceding claims, wherein the anti-C5 antibody comprises a heavy chain variable region set forth in SEQ ID NO:12 and a light chain variable region set forth in SEQ ID NO:8.

8. The method of any one of the preceding claims, wherein the anti-C5 antibody further comprises a heavy chain constant region set forth in SEQ ID NO:13.

9. The method of any one of the preceding claims, wherein the antibody comprises a heavy chain polypeptide comprising the amino acid sequence set forth in SEQ ID NO:14 and a light chain polypeptide comprising the amino acid sequence set forth in SEQ ID NO:11.

10. The method of any one of the preceding claims, wherein the anti-C5 antibody binds to human C5 at pH 7.4 and 25° C. with an affinity dissociation constant (K_{D}) that is in the range 0.1 nM≤K_{D}≤1 nM (e.g., about 0.5 nM).

11. The method of any one of the preceding claims, wherein the anti-C5 antibody binds to human C5 at pH 6.0 and 25° C. with a K_{D}≥10 nM (e.g., about 22 nM).

12. The method of any one of the preceding claims, wherein the anti-C5 antibody is administered to a patient weighing ≥40 to <60 kg once on Day 1 at a dose of 2400 mg, followed by a dose of 3000 mg at Week 2 and once every eight weeks thereafter.

13. The method of any one of the preceding claims, wherein the anti-C5 antibody is administered to a patient weighing ≥60<100 kg once on Day 1 at a dose of 2700 mg, followed by a dose of 3900 mg at Week 2 and once every eight weeks thereafter.

14. The method of any one of the preceding claims, wherein the anti-C5 antibody is administered to a patient weighing ≥100 kg once on Day 1 at a dose of 3000 mg, followed by a dose of 5400 mg at Week 2 and once every eight weeks thereafter to a patient weighing ≥100 kg.

15. The method of any one of claims 1-2 and 4-11, wherein the anti-C5 antibody is administered to a patient weighing ≥40 to <60 kg:
(a) once on Day 1 at a dose of 2400 mg, followed by a dose of 3000 mg at Week 2 and once every eight weeks thereafter; and
(b) once on Day 183 at a dose of 900 mg, followed by a dose of 3000 mg on Day 197 and once every eight weeks thereafter.

16. The method of any one of claims 1-2 and 4-11, wherein the anti-C5 antibody is administered to a patient weighing ≥60<100 kg:
(a) once on Day 1 at a dose of 2700 mg, followed by a dose of 3900 mg at Week 2 and once every eight weeks thereafter; and
(b) once on Day 183 at a dose of 900 mg, followed by a dose of 3900 mg on Day 197 and once every eight weeks thereafter.

17. The method of any one of claims 1-2, and 4-11, wherein the anti-C5 antibody is administered to a patient weighing ≥100 kg:
(a) once on Day 1 at a dose of 3000 mg, followed by a dose of 5400 mg at Week 2 and once every eight weeks thereafter; and
(b) once on Day 183 at a dose of 900 mg, followed by a dose of 5400 mg on Day 197 and once every eight weeks thereafter.

18. The method of any one of the preceding claims, wherein the treatment maintains a serum trough concentration of the anti-C5 antibody of 100 μg/mL or greater.

19. The method of any one of the preceding claims, wherein the treatment maintains a serum trough concentration of the anti-C5 antibody of 200 μg/mL or greater.

20. The method of any one of the preceding claims, wherein the anti-C5 antibody is formulated for intravenous administration.

21. The method of any one of claims 3, 6-14, and 18-20, wherein the LN patient has been previously treated with a background therapy comprising an immunosuppressant, e.g., corticosteroids and mycophenolate mofetil and/or the method of any one of claims 4-11 and 15-20, wherein the IgAN patient has previously been treated with a background therapy comprising renin-angiotensin system (RAS) inhibiting medication.

22. The method of claim 21, wherein the RAS is an angiotensin-converting enzyme (ACE) inhibitor or angiotensin II receptor blocker (ARB).

23. The method of any one of the preceding claims, wherein the treatment results in a shift towards normal levels of one or more renal injury biomarkers selected from the group consisting of CD163, MCP-1, and EGF.

24. The method of any one of the preceding claims, wherein the treatment results in a shift towards normal levels of one or more biomarkers selected from the group consisting of sC5b-9, Factor Ba, Factor Bb, C5a, C3c, C3, C4d, CD68, properdin, complement component 9 [C9], C1q, C5aR, and creatinine.

25. The method of any one of the preceding claims, wherein the treatment results in a change in Estimated glomerular filtration rate (eGFR) compared to baseline.

26. The method of any one of the preceding claims, wherein the treatment results in a change in serum albumin compared to baseline.

27. The method of any one of the preceding claims, wherein the treatment results in a reduction in proteinuria compared to baseline.

28. The method of any one of the preceding claims, wherein the patient has an estimated glomerular filtration rate (eGFR)≥30 mL/min/1.73m2 and proteinuria prior to treatment.

29. The method of any one of claims 27-28, wherein proteinuria is a urine protein to creatinine ratio (UPCR)≥1 g/g from one 24-hr urine collection.

30. The method of any one of claims 27-28, wherein proteinuria is a mean protein≥1 g/24-hr from 2 valid 24-hr collections.

31. The method of any one of claims 27-30, wherein there is a 30%, 40%, 50%, 60%, 70%, 80%, 90%, or 95% reduction in proteinuria after treatment compared to baseline.

32. The method of any one of claims 27-31, wherein the reduction in proteinuria occurs at 6 weeks, 8 weeks, 10 weeks, 12 weeks, 14 weeks, 16 weeks, 18 weeks, 20 weeks, 22 weeks, 24 weeks, 26 weeks, 28 weeks, or 30 weeks after treatment compared to baseline.

33. The method of any one of claims 27-32, wherein proteinuria is measured by a complete 24-hour urine collection.

34. The method of any one of claims 3, 6-14, and 18-20, wherein the treatment results in a reduction or cessation in one or more of the following symptoms compared to baseline: foamy urine, proteinuria, edema, high blood pressure, kidney inflammation, kidney impairment, joint pain, joint swelling, muscle pain, fever with no known cause, high levels of creatinine in the blood, and/or a red rash.

35. The method of any one of claims 3, 6-14, and 18-20, wherein the LN patient has an active flare prior to treatment.

36. The method of any one of claims 3, 6-14, and 18-20, wherein the treatment results in a Complete Renal Response (CRR).

37. The method of claim 36, wherein the CRR comprises:
(a) a decrease in mean urine protein-to-creatinine ratio (UPCR) to ≤0.5 g/g based on two 24-hour urine collections;
(b) an Estimated glomerular filtration rate (eGFR)≥60 mL/min/1.73 m2 or no eGFR reduction≥20% from the baseline value based on mean of 2 values; and
(c) no treatment failure.

38. The method of any one of claims 13, 6-14, and 18-20, wherein the treatment results in a Partial Renal Response (PRR).

39. The method of claim 38, wherein the PRR comprises:
(a) a decrease in UPCR>50% compared to the baseline value based on mean of two 24 hour urine collections;
(b) an Estimated glomerular filtration rate (eGFR)≥60 mL/min/1.73 m2 or no eGFR reduction≥20% from the baseline value based on mean of 2 values; and
(c) no treatment failure.

40. The method of any one of claims 3, 6-14, and 18-20, wherein the treatment prevents a renal flare, wherein:
(a) renal flare for a patient who has achieved CRR is reproducible recurrence of proteinuria≥1 g/g; and
(b) renal flare for a patient who has not achieved CRR is:
(i) a reproducible increase of serum creatinine>25% higher than baseline or above the upper limit of normal, including any one of the following:
a. reproducible proteinuria≥75% higher than baseline;
b. worsening active urinary sediment compared to baseline as defined by an increase of ≥5 RBCs/high power field (hpf) or new RBC casts (based on local laboratory results from at least 2 samples); and/or
c. kidney biopsy newly conducted since the biopsy used for eligibility demonstrating LN Class III or IV activity;
(ii) a reproducible doubling of the UPCR from a 24 hour urine collection compared with the lowest previous value obtained after the first dose of the anti-C5 antibody, or antigen binding fragment thereof.

41. The method of any one of claims 13, 6-14, and 18-20, wherein the treatment prevents a Extrarenal SLE Flare, wherein the Extrarenal SLE Flare comprises an increase in Systemic Lupus Erythematosus Disease Activity Index Safety of Estrogen in Lupus Erythematosus National Assessment (SELENA) Modification (SLEDAI-2K)≥4 points that is not accounted for by proteinuria, hematuria, urinary cellular casts, hypocomplementemia, or an increase in anti-double-stranded DNA (anti-dsDNA) antibody level.

42. The method of any one of claims 4-11 and 15-20, wherein the treatment results in Partial Remission (PR).

43. The method of claim 42, wherein PR comprises mean proteinuria<1 g/24-hours based on 2 valid 24-hour urine collections.

44. The method of any one of claims 4-11 and 15-20, wherein the treatment results in a reduction or cessation in one or more of the following symptoms compared to baseline: hematuria, dark brown or cola colored urine, edema, flank pain, hypertension, foamy urine, and/or proteinuria.

45. The method of any one of the preceding claims, wherein the treatment results in an improvement in the patient's quality of life, as assessed by European Quality of Life Health 5-item questionnaire dimensions 5 level (EQ-5D-5L) and/or Short Form (36) Health Survey (SF-36) total score.

46. The method of any one of claims 3, 6-14, and 18-20, wherein the treatment results in an improvement in the patient's quality of life, as assessed by Functional Assessment of Chronic Therapy (FACIT)-Fatigue score.

47. The method of any one of claims 3, 6-14, and 18-20, wherein the treatment further comprises administering one or more of the following:
(a) pneumocystis pneumonia prophylaxis;
(b) an antimalarial agent; and/or
(c) an agent to treat osteoporosis.

48. The method of claim 47, wherein the antimalarial agent is hydroxychloroquine.

49. The method of claim 47, wherein the agent to treat osteoporosis is selected from the group consisting of calcium carbonate or citrate, Vitamin D, and bisphosphonates.

50. The method of any one of the preceding claims, wherein the treatment results in terminal complement inhibition.

51. The method of any one of the preceding claims, wherein the treatment results in a reduction in adverse events.

52. The method of any one of the preceding claims, wherein the human patient is an adult patient.

53. A kit for treating lupus nephritis (LN) in a human patient, the kit comprising:
(a) a dose of an anti-C5 antibody, or antigen binding fragment thereof, comprising CDR1, CDR2 and CDR3 domains of the heavy chain variable region having the sequence set forth in SEQ ID NO:12, and CDR1, CDR2 and CDR3 domains of the light chain variable region having the sequence set forth in SEQ ID NO:8; and
(b) instructions for using the anti-C5 antibody, or antigen binding fragment thereof, in the method of any one of claims 1, 4-12, 16-18, and 21-27.

54. A kit for treating immunoglobulin A nephropathy (IgAN) in a human patient, the kit comprising:
(a) a dose of an anti-C5 antibody, or antigen binding fragment thereof, comprising CDR1, CDR2 and CDR3 domains of the heavy chain variable region having the sequence set forth in SEQ ID NO:12, and CDR1, CDR2 and CDR3 domains of the light chain variable region having the sequence set forth in SEQ ID NO:8; and
(b) instructions for using the anti-C5 antibody, or antigen binding fragment thereof, in the method of any one of claims 2-27.

55. An anti-C5 antibody, or antigen binding fragment thereof, comprising CDR1, CDR2 and CDR3 domains of the heavy chain variable region having the sequence set forth in SEQ ID NO:12, and CDR1, CDR2 and CDR3 domains of the light chain variable region having the sequence set forth in SEQ ID NO:8, wherein the anti-C5 antibody, or antigen binding fragment thereof, is administered, optionally together with a background therapy for treating C5-mediated glomerulonephritis (GN):
(a) once on Day 1 at a dose of 2400 mg, followed by a dose of 3000 mg at Week 2 and once every eight weeks thereafter to a patient weighing ≥40 to <60 kg;
(b) once on Day 1 at a dose of 2700 mg, followed by a dose of 3900 mg at Week 2 and once every eight weeks thereafter to a patient weighing ≥60 to <100 kg; or
(c) once on Day 1 at a dose of 3000 mg, followed by a dose of 5400 mg at Week 2 and once every eight weeks thereafter to a patient weighing ≥100 kg.

56. An anti-C5 antibody, or antigen binding fragment thereof, comprising CDR1, CDR2 and CDR3 domains of the heavy chain variable region having the sequence set forth in SEQ ID NO:12, and CDR1, CDR2 and CDR3 domains of the light chain variable region having the sequence set forth in SEQ ID NO:8, wherein the anti-C5 antibody, or antigen binding fragment thereof, is administered, optionally together with a background therapy for treating C5-mediated glomerulonephritis (GN):
(a) once on Day 1 at a dose of 2400 mg, followed by a dose of 3000 mg at Week 2 and once every eight weeks thereafter and once on Day 183 at a dose of 900 mg, followed by a dose of 3000 mg on Day 197 and once every eight weeks thereafter, to a patient weighing ≥40 to <60 kg;
(b) once on Day 1 at a dose of 2700 mg, followed by a dose of 3900 mg at Week 2 and once every eight weeks thereafter and once on Day 183 at a dose of 900 mg, followed by a dose of 3900 mg on Day 197 and once every eight weeks thereafter, to a patient weighing ≥60 to <100 kg; or
(c) once on Day 1 at a dose of 3000 mg, followed by a dose of 5400 mg at Week 2 and once every eight weeks thereafter and once on Day 183 at a dose of 900 mg, followed by a dose of 5400 mg on Day 197 and once every eight weeks thereafter, to a patient weighing ≥100 kg.

57. The antibody of claim 55 or 56, wherein the antibody is determined to be safe, tolerable, efficacious and sufficiently non-immunogenic after multiple IV doses in human patients and wherein the optional background therapy comprises (a) background therapy for treating lupus nephritis (LN) comprising an immunosuppressant, e.g., a corticosteroid and/or mycophenolate mofetil or (b) background therapy for treating IgA nephropathy (IgAN) comprising renin-angiotensin system (RAS) inhibiting medication.


SEQUENCE LISTING (50 sequences)

SEQ ID NO: 1 - protein, 10 aa, Artificial Sequence
  source: /note=“Description of Artificial Sequence Synthetic peptide”
GYIFSNYWIQ

SEQ ID NO: 2 - protein, 17 aa, Artificial Sequence
  source: /note=“Description of Artificial Sequence Synthetic peptide”
EILPGSGSTEYTENFKD

SEQ ID NO: 3 - protein, 13 aa, Artificial Sequence
  source: /note=“Description of Artificial Sequence Synthetic peptide”
YFFGSSPNWYFDV

SEQ ID NO: 4 - protein, 11 aa, Artificial Sequence
  source: /note=“Description of Artificial Sequence Synthetic peptide”
GASENIYGALN

SEQ ID NO: 5 - protein, 7 aa, Artificial Sequence
  source: /note=“Description of Artificial Sequence Synthetic peptide”
GATNLAD

SEQ ID NO: 6 - protein, 9 aa, Artificial Sequence
  source: /note=“Description of Artificial Sequence Synthetic peptide”
QNVLNTPLT

SEQ ID NO: 7 - protein, 122 aa, Artificial Sequence [sequence omitted]

SEQ ID NO: 8 - protein, 107 aa, Artificial Sequence [sequence omitted]

SEQ ID NO: 9 - protein, 326 aa, Artificial Sequence
  source: /note=“Description of Artificial Sequence Synthetic polypeptide”
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 447 aa, Artificial Sequence
  source: /note=“Description of Artificial Sequence Synthetic polypeptide”
QVQLVQSGAEVKKPGASVKVSCKASGYIFSNYWIQWVRQAPGQGLEWMGEILPGSGSTEYTENFKDRVTMTRDTSTSTVYMELSSLRSEDTAVYYCARYFFGSSPNWYFDVWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 11 - protein, 214 aa, Artificial Sequence [sequence omitted]

SEQ ID NO: 12 - protein, 122 aa, Artificial Sequence [sequence omitted]

SEQ ID NO: 13 - protein, 326 aa, Artificial Sequence
  source: /note=“Description of Artificial Sequence Synthetic polypeptide”
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGK

SEQ ID NO: 14 - protein, 448 aa, Artificial Sequence
  source: /note=“Description of Artificial Sequence Synthetic polypeptide”
QVQLVQSGAEVKKPGASVKVSCKASGHIFSNYWIQWVRQAPGQGLEWMGEILPGSGHTEYTENFKDRVTMTRDTSTSTVYMELSSLRSEDTAVYYCARYFFGSSPNWYFDVWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGK

SEQ ID NO: 15 - protein, 326 aa, Artificial Sequence
  source: /note=“Description of Artificial Sequence Synthetic polypeptide”
ASTKGPSVFPLAPCSRSTSESTAALGCLVKDYFPEPVTVSWNSGALTSGVHTFPAVLQSSGLYSLSSVVTVTSSNFGTQTYTCNVDHKPSNTKVDKTVERKCCVECPPCPAPPVAGPSVFLFPPKPKDTLYITREPEVTCVVVDVSHEDPEVQFNWYVDGMEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 16 - protein, 448 aa, Artificial Sequence
  source: /note=“Description of Artificial Sequence Synthetic polypeptide”
QVQLVQSGAEVKKPGASVKVSCKASGYIFSNYWIQWVRQAPGQGLEWMGEILPGSGSTEYTENFKDRVTMTRDTSTSTVYMELSSLRSEDTAVYYCARYFFGSSPNWYFDVWGQGTLVTVSSASTKGPSVFPLAPCSRSTSESTAALGCLVKDYFPEPVTVSWNSGALTSGVHTFPAVLQSSGLYSLSSVVTVTSSNFGTQTYTCNVDHKPSNTKVDKTVERKCCVECPPCPAPPVAGPSVFLFPPKPKDTLYITREPEVTCVVVDVSHEDPEVQFNWYVDGMEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 17 - protein, 11 aa, Artificial Sequence
  source: /note=“Description of Artificial Sequence Synthetic peptide”
GASENIYHALN

SEQ ID NO: 18 - protein, 17 aa, Artificial Sequence
  source: /note=“Description of Artificial Sequence Synthetic peptide”
EILPGSGHTEYTENFKD

SEQ ID NO: 19 - protein, 10 aa, Artificial Sequence
  source: /note=“Description of Artificial Sequence Synthetic peptide”
GHIFSNYWIQ

SEQ ID NO: 20 - protein, 448 aa, Artificial Sequence
  source: /note=“Description of Artificial Sequence Synthetic polypeptide”
QVQLVQSGAEVKKPGASVKVSCKASGHIFSNYWIQWVRQAPGQGLEWMGEILPGSGHTEYTENFKDRVTMTRDTSTSTVYMELSSLRSEDTAVYYCARYFFGSSPNWYFDVWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 21 - protein, 5 aa, Artificial Sequence
  source: /note=“Description of Artificial Sequence Synthetic peptide”
SYAIS

SEQ ID NO: 22 - protein, 17 aa, Artificial Sequence
  source: /note=“Description of Artificial Sequence Synthetic peptide”
GIGPFFGTANYAQKFQG

SEQ ID NO: 23 - protein, 7 aa, Artificial Sequence
  source: /note=“Description of Artificial Sequence Synthetic peptide”
DTPYFDY

SEQ ID NO: 24 - protein, 11 aa, Artificial Sequence
  source: /note=“Description of Artificial Sequence Synthetic peptide”
SGDSIPNYYVY

SEQ ID NO: 25 - protein, 7 aa, Artificial Sequence
  source: /note=“Description of Artificial Sequence Synthetic peptide”
DDSNRPS

SEQ ID NO: 26 - protein, 11 aa, Artificial Sequence
  source: /note=“Description of Artificial Sequence Synthetic peptide”
QSFDSSLNAEV

SEQ ID NO: 27 - protein, 116 aa, Artificial Sequence
  source: /note=“Description of Artificial Sequence Synthetic polypeptide”
QVQLVQSGAEVKKPGSSVKVSCKASGGTFSSYAISVWRQAPGQGLEWMGGIGPFFGTANYAQKFQGRVTITADESTSTAYMELSSLRSEDTAVYYCARDTPYFDYWGQGTLVTVSS

SEQ ID NO: 28 - protein, 108 aa, Artificial Sequence
  source: /note=“Description of Artificial Sequence Synthetic polypeptide”
DIELTQPPSVSVAPGQTARISCSGDSIPNYYVYWYQQKPGQAPVLVIYDDSNRPSGIPERFSGSNSGNTATLTISGTQAEDEADYYCQSFDSSLNAEVFGGGTKLTVL

SEQ ID NO: 29 - protein, 4 aa, Artificial Sequence
  source: /note=“Description of Artificial Sequence Synthetic peptide”
NYIS

SEQ ID NO: 30 - protein, 17 aa, Artificial Sequence
  source: /note=“Description of Artificial Sequence Synthetic peptide”
IIDPDDSYTEYSPSFQG

SEQ ID NO: 31 - protein, 8 aa, Artificial Sequence
  source: /note=“Description of Artificial Sequence Synthetic peptide”
YEYGGFDI

SEQ ID NO: 32 - protein, 11 aa, Artificial Sequence
  source: /note=“Description of Artificial Sequence Synthetic peptide”
SGDNIGNSYVH

SEQ ID NO: 33 - protein, 7 aa, Artificial Sequence
  source: /note=“Description of Artificial Sequence Synthetic peptide”
KDNDRPS

SEQ ID NO: 34 - protein, 9 aa, Artificial Sequence
  source: /note=“Description of Artificial Sequence Synthetic peptide”
GTYDIESYV

SEQ ID NO: 35 - protein, 116 aa, Artificial Sequence
  source: /note=“Description of Artificial Sequence Synthetic polypeptide”
EVQLVQSGAEVKKPGESLKISCKGSGYSFTNYISWVRQMPGKGLEWMGIIDPDDSYTEYSPSFQGQVTISADKSISTAYLQWSSLKASDTAMYYCARYEYGGFDIWGQGTLVTVSS

SEQ ID NO: 36 - protein, 106 aa, Artificial Sequence
  source: /note=“Description of Artificial Sequence Synthetic polypeptide”
SYELTQPPSVSVAPGQTARISCSGDNIGNSYVHWYQQKPGQAPVLVIYKDNDRPSGIPERFSGSNSGNTATLTISGTQAEDEADYYCGTYDIESYVFGGGTKLTVL

SEQ ID NO: 37 - protein, 6 aa, Artificial Sequence
  source: /note=“Description of Artificial Sequence Synthetic peptide”
SSYYVA

SEQ ID NO: 38 - protein, 17 aa, Artificial Sequence
  source: /note=“Description of Artificial Sequence Synthetic peptide”
AIYTGSGATYKASWAKG

SEQ ID NO: 39 - protein, 13 aa, Artificial Sequence
  source: /note=“Description of Artificial Sequence Synthetic peptide”
DGGYDYPTHAMHY

SEQ ID NO: 40 - protein, 11 aa, Artificial Sequence
  source: /note=“Description of Artificial Sequence Synthetic peptide”
QASQNIGSSLA

SEQ ID NO: 41 - protein, 7 aa, Artificial Sequence
  source: /note=“Description of Artificial Sequence Synthetic peptide”
GASKTHS

SEQ ID NO: 42 - protein, 12 aa, Artificial Sequence
  source: /note=“Description of Artificial Sequence Synthetic peptide”
QSTKVGSSYGNH

SEQ ID NO: 43 - protein, 123 aa, Artificial Sequence
  source: /note=“Description of Artificial Sequence Synthetic polypeptide”
QVQLVESGGGLVQPGGSLRLSCAASGFTSHSSYYVAWVRQAPGKGLEWVGAIYTGSGATYKASWAKGRFTISKDTSKNQVVLTMTNMDPVDTATYYCASDGGYDYPTHAMHYWGQGTLVTVSS

SEQ ID NO: 44 - protein, 110 aa, Artificial Sequence
  source: /note=“Description of Artificial Sequence Synthetic polypeptide”
DVVMTQSPSSLSASVGDRVTITCQASQNIGSSLAWYQQKPGQAPRLLIYGASKTHSGVPSRFSGSGSGTDFTLTISSLQPEDVATYYCQSTKVGSSYGNHFGGGTKVEIK

SEQ ID NO: 45 - protein, 451 aa, Artificial Sequence
  source: /note=“Description of Artificial Sequence Synthetic polypeptide”
QVQLVESGGGLVQPGRSLRLSCAASGFTVHSSYYMAWVRQAPGKGLEWVGAIFTGSGAEYKAEWAKGRVTISKDTSKNQVVLTMTNMDPVDTATYYCASDAGYDYPTHAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELRRGPKVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHAHYTRKELSLSP

SEQ ID NO: 46 - protein, 217 aa, Artificial Sequence
  source: /note=“Description of Artificial Sequence Synthetic polypeptide”
DIQMTQSPSSLSASVGDRVTITCRASQGISSSLAWYQQKPGKAPKLLIYGASETESGVPSRFSGSGSGTDFTLTISSLQPEDFATYYCQNTKVGSSYGNTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 47 - protein, 120 aa, Artificial Sequence
  source: /note=“Description of Artificial Sequence Synthetic polypeptide”
QVQLQESGPGLVKPSETLSLTCTVSGDSVSSSYWTWIRQPPGKGLEWIGYIYYSGSSNYNPSLKSRATISVDTSKNQFSLKLSSVTAADTAVYYCAREGNVDTTMIFDYWGQGTLVTVSS

SEQ ID NO: 48 - protein, 107 aa, Artificial Sequence
  source: /note=“Description of Artificial Sequence Synthetic polypeptide”
AIQMTQSPSSLSASVGDRVTITCRASQGIRNDLGWYQQKPGKAPKLLIYAASSLQSGVPSRFAGRGSGTDFTLTISSLQPEDFATYYCLQDFNYPWTFGQGTKVEIK

SEQ ID NO: 49 - protein, 447 aa, Artificial Sequence
  source: /note=“Description of Artificial Sequence Synthetic polypeptide”
QVQLQESGPGLVKPSETLSLTCTVSGDSVSSSYWTWIRQPPGKGLEWIGYIYYSGSSNYNPSLKSRATISVDTSKNQFSLKLSSVTAADTAVYYCAREGNVDTTMIFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 50 - protein, 214 aa, Artificial Sequence
  source: /note=“Description of Artificial Sequence Synthetic polypeptide”
AIQMTQSPSSLSASVGDRVTITCRASQGIRNDLGWYQQKPGKAPKLLIYAASSLQSGVPSRFAGRGSGTDFTLTISSLQPEDFATYYCLQDFNYPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC